(19) United States
(12) Patent Application Publication
(10) Pub. No.: US 2006/0241145 A1
(43) Pub. Date: Oct. 26, 2006

(54) Piperidine derivative crystal, process for producing the same, and use

(76) Inventors: Yoshinori Ikeura, Osaka (JP); Tadatoshi Hashimoto, Osaka (JP); Junya Shirai, Osaka (JP); Yoshikawa Takeshi, Osaka-shi (JP); Hiroshi Nakatani, Osaka (JP); Mitsuhisa Yamano, Osaka (JP); Masahiro Mizuno, Osaka (JP); Hiroyuki Irie, Osaka (JP)

Correspondence Address: WENDEROTH, LIND & PONACK, L.L.P., 2033 K STREET N. W., SUITE 800, WASHINGTON, DC, 20006-1021, US

(21) Appl. No.: 11/407,209

(22) Filed: Apr. 20, 2006

(30) Foreign Application Priority Data
Apr. 21, 2005 (JP) 124334/2005

(51) Int. Cl. A61K 31/454; C07D 403/02

(52) U.S. Cl. 514/326; 546/210

(57) ABSTRACT

The present invention provides a piperidine derivative having antagonistic action for tachykinin receptors and the like, a crystal thereof, and an agent for the prophylaxis or treatment of diseases including lower urinary tract disease and the like, which contains the derivative. Specifically, the present invention provides an optically active compound represented by the formula (I): 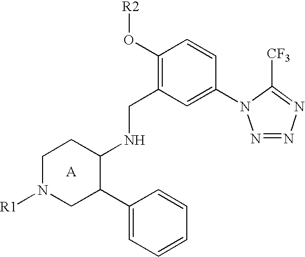 wherein each symbol is as defined in the specification, and a salt thereof.

### TECHNICAL FIELD

The present invention relates to a novel piperidine derivative having excellent antagonistic action for a tachykinin receptor, a crystal thereof, a production method thereof and use thereof.

### BACKGROUND ART

Tachykinin is a generic term for a group of neuropeptides. Substance P(SP), neurokinin-A and neurokinin-B are known in mammals, and these peptides are known to bind to the corresponding receptors (neurokinin-1, neurokinin-2 and neurokinin-3) that exist in a living body and thereby to exhibit various biological activities.

Of such neuropeptides, SP has the longest history and has been studied in detail. In 1931, the existence of SP in the extract from equine intestines was confirmed, and in 1971, its structure was determined. SP is a peptide consisting of 11 amino acids.

SP is broadly distributed over the central and peripheral nervous systems, and has various physiological activities such as vasodilation, enhancement of vascular extravasation, contraction of smooth muscles, excitation of neurons, salivation, enhancement of diuresis, immunological enhancement and the like, in addition to the function as a transmitter substance for primary sensory neurons. In particular, it is known that SP released from the terminal in the spinal (dorsal) horn due to a pain impulse transmits the information of pain to secondary neurons, and that SP released from the peripheral nerve terminal induces an inflammatory response in the receptor thereof. Thus, it is considered that SP is involved in various disorders (e.g., pain, headache, particularly migraine, Alzheimer's disease, multiple sclerosis, cardiovascular modulation, chronic inflammatory diseases such as chronic rheumatic arthritis, respiratory diseases including asthma and allergic rhinitis, intestinal inflammatory diseases including ulcerative colitis and Crohn's disease, ocular damage and ocular inflammatory diseases, proliferative vitreous retinopathy, irritable bowel syndrome, urinary frequency, psychosis, vomiting etc.) [see Physiological Reviews, Vol. 73, pp. 229-308 (1993); and Journal of Autonomic Pharmacology, Vol. 13, pp. 23-93 (1993)].

WO03/101964 describes a compound having antagonistic action for tachykinin receptors, which is represented by the formula: 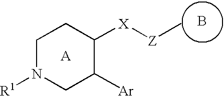
wherein Ar is an aryl group, an aralkyl group or an aromatic heterocyclic group, each of which optionally having substituent(s), R¹ is a hydrogen atom, a hydrocarbon group optionally having substituent(s), an acyl group or a heterocyclic group optionally having substituent(s), X is an oxygen atom or an imino group optionally having a substituent, Z is a methylene group optionally having substituent(s), ring A is a piperidine ring optionally further having substituent(s), and ring B is an aromatic ring optionally having substituent(s), provided when Z is a methylene group substituted by an oxo group, then R¹ is not a methyl group and when Z is a methylene group substituted by a methyl group, then ring B is an aromatic ring having substituent(s), or a salt thereof.

US-A-2005/0256164 describes a compound having antagonistic action for tachykinin receptors, which is represented by the formula: 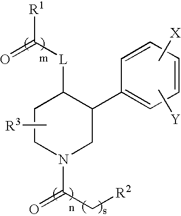
wherein m is 0 or 1; n is 0 or 1; s is 0 or 1; L is —O— or —N(R⁴)—; R¹ and R² are each independently hydrogen atom, aryl, heteroaryl, C₁₋₆ alkyl, heterocycloalkyl, C₁₋₆ alkylheterocycloalkyl, C₁₋₆ alkylheteroaryl, C₁₋₆ alkyl-O-aryl, C₁₋₆ alkylaryl, or —CH₂N(R⁴)(R⁵), wherein each of said heterocyloalkyl, C₁₋₆ alkylheterocycloalkyl, C₁₋₆ alkylheteroaryl, C₁₋₆ alkyl-O-aryl, aryl, C₁₋₆ alkylaryl, heteroaryl, and —CH₂N(R⁴)(R⁵), is optionally substituted with 1 to 3 substituents independently selected from X′, Y′ or Z′; R³ is hydrogen atom, CF₃, OH, or C₁₋₆ alkyl; R⁴ and R⁵ are each independently selected from hydrogen atom, C₁₋₆ alkyl or C₁₋₆ (C═O)R⁷; R⁷ is C₁₋₆ alkyl, OH, —CH₂N(R⁴)(R⁵) or —OR⁴; R⁸ and R⁹ are each independently C₁₋₆ alkyl; X, Y, X′, Y′ and Z′ are each independently selected from hydrogen atom, C₁₋₆ alkyl, C₁₋₆ alkyl-NR⁴R⁵, CF₃, OH, —O—C₁₋₆ alkyl, C₁₋₆ alkyl-C(═O)R⁷, aryl, heteroaryl, cycloalkyl, NO₂, C₁₋₆ alkylaryl, —O-aryl, halogen, CN, —CH₃N(R⁴)(R⁵), —C(═O)R⁷, —R⁶C(═O)R⁷ or —R⁶C(═O)N(R⁴)(R⁵); and R⁶ is a bond, —CH₂—, —O— or —NR⁴—, or a salt thereof.

### DISCLOSURE OF THE INVENTION

An object of the present invention is to provide a piperidine derivative having antagonistic action for tachykinin receptors etc. with a different chemical structure from the known compounds including the above-mentioned compounds, a crystal thereof, and an agent for the prophylaxis or treatment of diseases including lower urinary tract disease and the like comprising the derivative.

The present inventors have made extensive studies in consideration of the above-mentioned problem and, as a result, have found unexpectedly that piperidine derivatives represented by the formula (I) below or a salt thereof have an excellent antagonistic action for tachykinin receptors (particularly antagonistic action for SP receptors) as based on their peculiar chemical structures and are sufficiently satisfactory as pharmaceutical compositions. Particularly, they have succeeded in producing a crystal of N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide having high purity, high quality and low hygroscopicity, which is not denatured even after a long-term preservation under normal conditions and is extremely superior in stability, which resulted in the completion of the present invention.

Accordingly, the present invention provides
[1] An optically active compound represented by the formula (I): 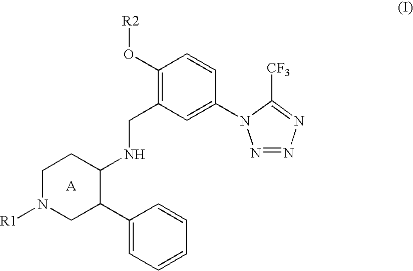
wherein
ring A is an optionally further substituted piperidine ring,
R1 is a hydrogen atom or a group represented by
R1′-C(═O)—

wherein R1′ is
(i) an optionally substituted 5- or 6-membered nitrogen-containing heterocyclic group,
(ii) an optionally substituted C₁₋₆ alkyl group, or
(iii) an optionally substituted C₁₋₆ alkoxy group, and
   R2 is a hydrogen atom, an optionally substituted C₁₋₃ alkyl group, or a C₃₋₆ cycloalkyl group, except cis-1-(methoxyacetyl)-N-[2-methoxy-5-[5-(trifluoromethyl)-1H-tetrazol-1-yl]benzyl]-3-phenyl-4-piperidinamine and cis-1-[(1-acetyl-4-piperidinyl)carbonyl]-N-[2-methoxy-5-[5-(trifluoromethyl)-1H-tetrazol-1-yl]benzyl]-3-phenyl-4-piperidinamine,
   or a salt thereof (hereinafter to be referred to as compound (I));
   [2] the compound of the above-mentioned [1], wherein R1 is a hydrogen atom or a group represented by R1′-C(═O)—

wherein R1′ is
(i) an optionally substituted 5- or 6-membered nitrogen-containing heterocyclic group,
(ii) an optionally substituted C₁₋₆ alkyl group, or
(iii) an optionally substituted C₁₋₆ alkoxy group,
except a methoxymethyl group and a 1-acetylpiperidin-4-yl group, and
   R2 is a hydrogen atom, a C₁₋₃ alkyl group or a C₃₋₆ cycloalkyl group;
   [3] the compound of the above-mentioned [1], which is a compound represented by the formula (I-A): 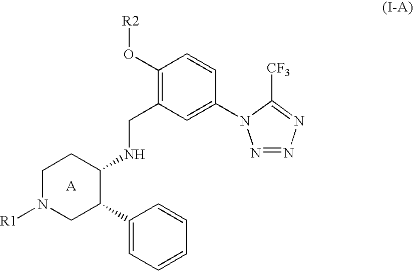
   wherein each symbol is as defined in the above-mentioned [1] (hereinafter to be referred to as compound (I-A));
   [4] the compound of the above-mentioned [1], which is a compound represented by the formula (Ia-A): 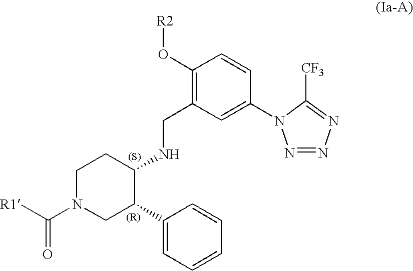
   wherein
   R1′ is
   (i) a 5- or 6-membered nitrogen-containing heterocyclic group optionally having C₁₋₆ alkylsulfonyl group(s),
   (ii) a C₁₋₆ alkyl group optionally having 1 to 3 substituents selected from

(1) —NR3R4

wherein

R3 is
(a) a hydrogen atom or
(b) a C₁₋₆ alkyl group optionally having oxo group(s), and

R4 is a hydrogen atom, or

R3 and R4 in combination optionally form a 5- to 7-membered ring optionally having oxo group(s),

(2) a C₁₋₆ alkylsulfonyl group,

(3) a hydroxy group and

(4) an oxo group, or

(iii) a C₁₋₆ alkoxy group, and

R2 is a hydrogen atom, methyl or trifluoromethyl (hereinafter to be referred to as compound (Ia-A));

[5] N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide or a salt thereof;

[6] a crystal of N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide or a salt thereof;

[7] a crystal of N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide;

[8] the crystal of the above-mentioned [7], which has a melting point of not less than 90° C.;

[9] the crystal of the above-mentioned [7], wherein the melting point is about 107° C. to about 119° C.;

[10] the crystal of the above-mentioned [7], wherein the melting point is about 124° C. to about 134° C.;

[11] the crystal of the above-mentioned [9], showing a diffraction pattern having characteristic peaks of lattice spacing (d value) at about 5.83, about 5.17, about 4.61, about 4.00 and about 3.40 angstroms by powder X-ray diffraction;

[12] the crystal of the above-mentioned [10], showing a diffraction pattern having characteristic peaks of lattice spacing (d value) at about 7.26, about 4.61, about 4.54, about 4.38 and about 3.63 angstroms by powder X-ray diffraction;

[13] a pharmaceutical agent comprising the compound of the above-mentioned [1];

[14] the pharmaceutical agent of the above-mentioned [13], which is a tachykinin receptor antagonist;

[15] the pharmaceutical agent of the above-mentioned [13], which is an agent for the prophylaxis or treatment of lower urinary tract disease, gastrointestinal disease or central nervous system disease;

[16] the pharmaceutical agent of the above-mentioned [13], which is an agent for the prophylaxis or treatment of lower urinary tract disease associated with overactive bladder and benign prostatic hyperplasia, pelvic visceral pain, lower urinary tract disease associated with chronic prostatitis, lower urinary tract disease associated with interstitial cystitis, irritable bowel syndrome, inflammatory bowel disease, vomiting, nausea, depression, anxiety neurosis, anxiety or sleep disorder (insomnia);

[17] a method for the prophylaxis or treatment of lower urinary tract disease, gastrointestinal disease or central nervous system disease in mammals, which comprises administering an effective amount of the compound of the above-mentioned [1] to said mammals;

[18] use of the compound of the above-mentioned [1], for the production of an agent for the prophylaxis or treatment of lower urinary tract disease, gastrointestinal disease or central nervous system disease;

[19] a method of producing the compound of the above-mentioned [4], which comprises subjecting a compound represented by the formula; 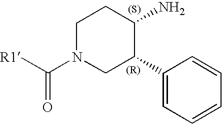
wherein each symbol is as defined in the above-mentioned [4], or a salt thereof, to reductive alkylation with a compound represented by the formula: 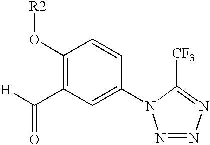
wherein each symbol is as defined in the above-mentioned [4], or a salt thereof;
[20] a method of producing the compound of the above-mentioned [5], which comprises subjecting N-{2-[(3R,4S)-4-amino-3-phenylpiperidin-1-yl]-2-oxoethyl}acetamide or a salt thereof to reductive alkylation with 2-methoxy-5-[5-(trifluoromethyl)-1H-tetrazol-1-yl]benzaldehyde or a salt thereof;
[21] a method of producing the crystal of the above-mentioned [9], which comprises bringing a solution of N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide or a salt thereof to supersaturation at less than 46° C. and performing crystal precipitation;
[22] a method of producing the crystal of the above-mentioned [10], which comprises bringing a solution of N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide or a salt thereof to supersaturation at not less than 46° C. and performing crystal precipitation;
[23] N-{2-[(3R,4S)-4-amino-3-phenylpiperidin-1-yl]-2-oxoethyl}acetamide or a salt thereof;
[24] a crystal of N-{2-[(3R,4S)-4-amino-3-phenylpiperidin-1-yl]-2-oxoethyl}acetamide methanesulfonate;
[25] a method of producing a compound represented by the formula: 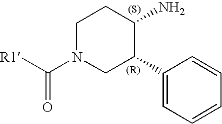
wherein R1′ is
(i) a 5- or 6-membered nitrogen-containing heterocyclic group optionally having C₁₋₆ alkylsulfonyl group(s),
(ii) a C₁₋₆ alkyl group optionally having 1 to 3 substituents selected from

(1) —NR3R4

wherein

R3 is
(a) a hydrogen atom or
(b) a C₁₋₆ alkyl group optionally having oxo group(s), and

R4 is a hydrogen atom, or

R3 and R4 in combination optionally form a 5- to 7-membered ring optionally having oxo group(s),

(2) a C₁₋₆ alkylsulfonyl group,

(3) a hydroxy group and

(4) an oxo group, or

(iii) a C₁₋₆ alkoxy group,

or a salt thereof, which comprises condensing a compound represented by the formula: 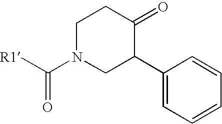
wherein R1′ is as defined above, with an optically active compound represented by the formula: 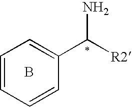
wherein ring B is an optionally fused benzene ring optionally having substituent(s), R2′ is a hydrocarbon group optionally having substituent(s), and * is an asymmetric center,
or a salt thereof, which is followed by hydrogenation and then hydrogenolysis;
[26] a method of producing the compound of the above-mentioned [23], which comprises condensing N-[2-oxo-2-(4-oxo-3-phenylpiperidin-1-yl)ethyl]acetamide with (S)-1-phenylethylamine or a salt thereof, hydrogenating the resulting compound to give N-[2-oxo-2-((3R,4S)-3-phenyl-4-{[(1S)-1-phenylethyl]amino}piperidin-1-yl)ethyl]acetamide or a salt thereof, and then hydrogenolyzing the compound;
[27] N-[2-oxo-2-(4-oxo-3-phenylpiperidin-1-yl)ethyl]acetamide;
[28] N-[2-oxo-2-((3R,4S)-3-phenyl-4-{[(1S)-1-phenylethyl]amino)piperidin-1-yl)ethyl]acetamide or a salt thereof;
and the like.

### EFFECT OF THE INVENTION

The compound (I) and a crystal thereof have high antagonistic action for a tachykinin receptor, particularly high antagonistic action for a substance P receptor, and low toxicity, and are superior in in vivo kinetics (absorbability, distribution, metabolism, excretion) by oral administration, efficacy expression and solubility. Accordingly, compound (I) and a crystal thereof are safe as pharmaceutical agents. Therefore, compound (I) and a crystal thereof are useful as pharmaceutical agents, such as tachykinin receptor antagonists, agents for lower urinary tract symptoms and the like.

### BEST MODE FOR EMBODYING THE INVENTION

R1 is a hydrogen atom or a group represented by R1′-C(═O)—. As used herein, R1 is (i) an optionally substituted 5- or 6-membered nitrogen-containing heterocyclic group, (ii) an optionally substituted C₁₋₆ alkyl group or (iii) an optionally substituted C₁₋₆ alkoxy group.

(i) As the “5- or 6-membered nitrogen-containing heterocyclic group” of the “optionally substituted 5- or 6-membered nitrogen-containing heterocyclic group”, a 5- or 6-membered nitrogen-containing aromatic heterocyclic group, a saturated or unsaturated 5- or 6-membered nitrogen-containing non-aromatic heterocyclic group and the like, each of which containing, besides carbon atom(s) and one or more nitrogen atoms, one or two kinds of 1 to 4 hetero atoms selected from an oxygen atom and a sulfur atom, can be mentioned.

As the above-mentioned “5- or 6-membered nitrogen-containing aromatic heterocyclic group”, for example, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, triazinyl and the like can be mentioned.

As the above-mentioned “5- or 6-membered nitrogen-containing non-aromatic heterocyclic group”, for example, pyrrolidinyl, piperidinyl, morpholinyl, thiomorpholinyl, piperazinyl, dihydropyridyl, tetrahydropyridyl, dihydropyrimidyl, tetrahydropyrimidyl and the like can be mentioned.

The “5- or 6-membered nitrogen-containing heterocyclic group” of the “optionally substituted 5- or 6-membered nitrogen-containing heterocyclic group” is preferably a 5- or 6-membered nitrogen-containing non-aromatic heterocyclic group, more preferably piperidinyl, pyrrolidinyl, tetrahydropyrimidinyl and the like.

As the substituent which the “5- or 6-membered nitrogen-containing heterocyclic group” of the “optionally substituted 5- or 6-membered nitrogen-containing heterocyclic group” may has, for example,

a halogen atom (e.g., fluorine, chlorine, bromine, iodine),

a lower alkyl group (e.g., a C₁₋₆ alkyl group such as methyl, ethyl, propyl, isopropyl, butyl, isobutyl, sec-butyl, tert-butyl, pentyl, hexyl and the like, etc.),

a cycloalkyl group (e.g., a C₃₋₆ cycloalkyl group such as cyclopropyl, cyclobutyl, cyclopentyl, cyclohexyl and the like, etc.),

a lower alkynyl group (e.g., a C₂₋₆ alkynyl group such as ethynyl, 1-propynyl, propargyl and the like, etc.),

a lower alkenyl group (e.g., a C₂₋₆ alkenyl group such as vinyl, allyl, isopropenyl, butenyl, isobutenyl and the like, etc.),

an aralkyl group (e.g., a C₇₋₁₁ aralkyl group such as benzyl, α-methylbenzyl, phenethyl and the like, etc.),

an aryl group (e.g., a C₆₋₁₀ aryl group such as phenyl, naphthyl and the like, etc., preferably phenyl group etc.),

a lower alkoxy group (e.g., a C₁₋₆ alkoxy group such as methoxy, ethoxy, propoxy, isopropoxy, butoxy, isobutoxy, sec-butoxy, tert-butoxy and the like, etc.),

an aryloxy group (e.g., a C₆₋₁₀ aryloxy group such as phenoxy and the like, etc.),

a lower alkanoyl group (e.g., formyl; a C₁₋₆ alkyl-carbonyl group such as acetyl, propionyl, butyryl, isobutyryl and the like, etc.),

an arylcarbonyl group (e.g., a C₆₋₁₀ aryl-carbonyl group such as benzoyl, naphthoyl and the like, etc.),

a lower alkanoyloxy group (e.g., formyloxy; a C₁₋₆ alkyl-carbonyloxy group such as acetyloxy, propionyloxy, butyryloxy, isobutyryloxy and the like, etc.),

an arylcarbonyloxy group (e.g., a C₆₋₁₀ aryl-carbonyloxy group such as benzoyloxy, naphthoyloxy and the like, etc.),

a carboxyl group,

a lower alkoxycarbonyl group (e.g., a C₁₋₆ alkoxy-carbonyl group such as methoxycarbonyl, ethoxycarbonyl, propoxycarbonyl, isopropoxycarbonyl, butoxycarbonyl, isobutoxycarbonyl, tert-butoxycarbonyl and the like, etc.),

an aralkyloxycarbonyl group (e.g., a C₇₋₁₁ aralkyloxy-carbonyl group such as benzyloxycarbonyl and the like, etc.),

a carbamoyl group,

a mono-, di- or tri-halogeno-lower alkyl group (e.g., a mono-, di- or tri-halogeno-C₁₋₄ alkyl group such as chloromethyl, dichloromethyl, trifluoromethyl, 2,2,2-trifluoroethyl and the like, etc.),

an oxo group,

an amidino group,

an imino group,

an amino group,

a mono-lower alkylamino group (e.g., a mono-C₁₋₄ alkylamino group such as methylamino, ethylamino, propylamino, isopropylamino, butylamino and the like, etc.),

a di-lower alkylamino group (e.g., a di-C₁₋₄ alkylamino group such as dimethylamino, diethylamino, dipropylamino, diisopropylamino, dibutylamino, methylethylamino and the like, etc.),

a 3- to 6-membered cyclic amino group optionally containing, besides carbon atom(s) and one nitrogen atom, 1 to 3 hetero atoms selected from an oxygen atom, a sulfur atom and a nitrogen atom (e.g., a 3- to 6-membered cyclic amino group such as aziridinyl, azetidinyl, pyrrolidinyl, pyrrolinyl, pyrrolyl, imidazolyl, pyrazolyl, imidazolidinyl, piperidinyl, morpholinyl, dihydropyridyl, pyridyl, N-methylpiperazinyl, N-ethylpiperazinyl and the like, etc.),

an alkylenedioxy group (e.g., a C₁₋₃ alkylenedioxy group such as methylenedioxy, ethylenedioxy and the like, etc.),

a hydroxy group,

a nitro group,

a cyano group,

a mercapto group,

a sulfo group,

a sulfino group,

a phosphono group,

a sulfamoyl group,

a mono-lower alkylsulfamoyl group (e.g., a mono-C₁₋₆ alkyl sulfamoyl group such as N-methylsulfamoyl, N-ethylsulfamoyl, N-propylsulfamoyl, N-isopropylsulfamoyl, N-butylsulfamoyl and the like, etc.),

a di-lower alkylsulfamoyl group (e.g., a di-C₁₋₆ alkylsulfamoyl group such as N,N-dimethylsulfamoyl, N,N-diethylsulfamoyl, N,N-dipropylsulfamoyl, N,N-dibutylsulfamoyl and the like, etc.),

a lower alkylthio group (e.g., a C₁₋₆ alkylthio group such as methylthio, ethylthio, propylthio, isopropylthio, butylthio, sec-butylthio, tert-butylthio and the like, etc.),

an arylthio group (e.g., a C₆₋₁₀ arylthio group such as phenylthio, naphthylthio and the like, etc.),

a lower alkylsulfinyl group (e.g., a C₁₋₆ alkylsulfinyl group such as methylsulfinyl, ethylsulfinyl, propylsulfinyl, butylsulfinyl and the like, etc.),

an arylsulfinyl group (e.g., a C₆₋₁₀ arylsulfinyl group such as phenylsulfinyl, naphthylsulfinyl and the like, etc.),

a lower alkylsulfonyl group (e.g., a C₁₋₆ alkylsulfonyl group such as methylsulfonyl, ethylsulfonyl, propylsulfonyl, butylsulfonyl and the like, etc.),

an arylsulfonyl group (e.g., a C₆₋₁₀ arylsulfonyl group such as phenylsulfonyl, naphthylsulfonyl and the like, etc.) and the like can be mentioned.

The “5- or 6-membered nitrogen-containing heterocyclic group” of the “optionally substituted 5- or 6-membered nitrogen-containing heterocyclic group” may have 1 to 5, preferably 1 to 3, substituents mentioned above at substitutable positions on the heterocyclic group. When the number of the substituents is not less than 2, the respective substituents may be the same or different.

The “optionally substituted 5- or 6-membered nitrogen-containing heterocyclic group” is preferably a 5- or 6-membered nitrogen-containing heterocyclic group optionally having a C₁₋₆ alkylsulfonyl group, a C₁₋₆ alkyl group, an oxo group, a C₁₋₆ alkyl-carbonyl group and the like, particularly preferably a 5- or 6-membered nitrogen-containing heterocyclic group optionally having a C₁₋₆ alkylsulfonyl group.

(ii) As the “C₁₋₆ alkyl group” of the “optionally substituted C₁₋₆ alkyl group”, for example, a C₁₋₆ alkyl group such as methyl, ethyl, propyl, isopropyl, butyl, isobutyl, sec-butyl, tert-butyl, pentyl and hexyl and the like, and the like can be mentioned.

As the substituent which the “C₁₋₆ alkyl group” of the “optionally substituted C₁₋₆ alkyl group” may have, for example,

a halogen atom (e.g., fluorine, chlorine, bromine, iodine),

a nitro group,

a cyano group,

a hydroxy group,

an optionally halogenated lower alkyl group (e.g., an optionally halogenated C₁₋₆ alkyl group such as methyl, chloromethyl, difluoromethyl, trichloromethyl, trifluoromethyl, ethyl, 2-bromoethyl, 2,2,2-trifluoroethyl, pentafluoroethyl, propyl, 3,3,3-trifluoropropyl, isopropyl, butyl, isobutyl, sec-butyl, tert-butyl, 4,4,4-trifluorobutyl, pentyl, isopentyl, neopentyl, 5,5,5-trifluoropentyl, hexyl, 6,6,6-trifluorohexyl and the like, etc.),

a cycloalkyl group (e.g., a C₃₋₆ cycloalkyl group such as cyclopropyl, cyclobutyl, cyclopentyl, cyclohexyl and the like, etc.),

a lower alkoxy group (e.g., a C₁₋₆ alkoxy group such as methoxy, ethoxy, propoxy, isopropoxy, butoxy, isobutoxy, pentyloxy, hexyloxy and the like, etc.),

an amino group,

a mono-lower alkylamino group (e.g., a mono-C₁₋₆ alkylamino group such as methylamino, ethylamino and the like, etc.),

a di-lower alkylamino group (e.g., a di-C₁₋₆ alkylamino group such as dimethylamino, diethylamino and the like, etc.),

a carboxyl group,

a lower alkylcarbonyl group (e.g., a C₁₋₆ alkyl-carbonyl group such as acetyl, propionyl and the like, etc.),

a lower alkoxycarbonyl group (e.g., a C₁₋₆ alkoxy-carbonyl group such as methoxycarbonyl, ethoxycarbonyl, propoxycarbonyl, butoxycarbonyl and the like, etc.),

a carbamoyl group,

a thiocarbamoyl group,

a mono-lower alkylcarbamoyl group (e.g., a mono-C₁₋₆ alkyl-carbamoyl group such as methylcarbamoyl, ethylcarbamoyl and the like, etc.),

a di-lower alkylcarbamoyl group (e.g., a di-C₁₋₆ alkyl-carbamoyl group such as dimethylcarbamoyl, diethylcarbamoyl and the like, etc.),

an arylcarbamoyl group (e.g., a C₆₋₁₀ aryl-carbamoyl group such as phenylcarbamoyl, naphthylcarbamoyl and the like, etc.),

an aryl group (e.g., a C₆₋₁₀ aryl group such as phenyl, naphthyl and the like, etc.),

an aryloxy group (e.g., a C₆₋₁₀ aryloxy group such as phenyloxy, naphthyloxy and the like, etc.),

a lower alkylcarbonylamino group optionally substituted by 1 to 3 substituents selected from a halogen atom and a hydroxy group (e.g., a C₁₋₆ alkyl group-carbonylamino group optionally substituted by 1 to 3 substituents selected from a halogen atom and a hydroxy group such as acetylamino, trifluoroacetylamino, ethylcarbonylamino, 2-hydroxyacetylamino and the like, etc.),

an oxo group,

a 5- to 7-membered heterocyclic group,

a formylamino group,

a N-lower alkyl-N-formylamino group (e.g., a N—C₁₋₆ alkyl-N-formylamino group such as formylmethylamino, ethylformylamino and the like, etc.),

an ureido group,

a lower alkylthio group (e.g., a C₁₋₆ alkylthio group such as methylthio, ethylthio, propylthio, isopropylthio, butylthio, sec-butylthio, tert-butylthio and the like, etc.),

a lower alkylsulfinyl group (e.g., a C₁₋₆ alkylsulfinyl group such as methylsulfinyl, ethylsulfinyl, propylsulfinyl, butylsulfinyl and the like, etc.),

a lower alkylsulfonyl group (e.g., a C₁₋₆ alkylsulfonyl group such as methylsulfonyl, ethylsulfonyl, propylsulfonyl, butylsulfonyl and the like, etc.) and the like can be mentioned.

As the “5- to 7-membered heterocyclic group” which is the “substituent” for the “optionally substituted C₁₋₆ alkyl group”, for example, a 5- to 7-membered aromatic heterocyclic group, a saturated or unsaturated 5- to 7-membered non-aromatic heterocyclic group and the like, each of which containing, besides carbon atom(s), one or two kinds of 1 to 4 hetero atoms selected from a nitrogen atom, an oxygen atom and a sulfur atom, can be mentioned. The “5- to 7-membered heterocyclic group” optionally has substituents such as an oxo group and the like.

As the “5- to 7-membered aromatic heterocyclic group”, for example, furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, triazinyl and the like can be mentioned.

As the above-mentioned “5- to 7-membered non-aromatic heterocyclic group”, for example, pyrrolidinyl, tetrahydrofuryl, tetrahydrothienyl, piperidinyl, tetrahydropyranyl, morpholinyl, thiomorpholinyl, piperazinyl and the like can be mentioned.

These non-aromatic heterocyclic groups are optionally fused with other aromatic or non-aromatic homocyclic ring or heterocycle.

The “C₁₋₆ alkyl group” of the “optionally substituted C₁₋₆ alkyl group” may have 1 to 5, preferably 1 to 3, substituents mentioned above at substitutable positions on the C₁₋₆ alkyl group. When the number of the substituents is not less than 2, the respective substituents may be the same or different.

The “optionally substituted C₁₋₆ alkyl group” is preferably a C₁₋₆ alkyl group optionally having 1 to 3 substituents selected from

(1) —NR3R4

wherein
R3 is
   (a) a hydrogen atom or
   (b) a C₁₋₆ alkyl group optionally having 1 to 3 substituents selected from an oxo group, a hydroxy group and the like, and
R4 is a hydrogen atom or a C₁₋₆ alkyl group, or
R3 and R4 in combination optionally form a 5- to 7-membered ring optionally having oxo group(s),
   (2) a C₁₋₆ alkylsulfonyl group,
   (3) a hydroxy group,
   (4) an oxo group,
   (5) an alkylthio group,
   (6) an alkylsulfinyl group,
   (7) an ureido group
   and the like.

Here, as the 5- to 7-membered ring formed by R3 and R4, a ring constituting the “5- to 7-membered heterocyclic group”, which is the “substituent” for the “optionally substituted C₁₋₆ alkyl group”, wherein the ring contains at least one nitrogen atom, can be mentioned. Preferred are pyrrolidine, tetrazole and the like.

It is more preferably a C₁₋₆ alkyl group optionally having 1 to 3 substituents selected from

(1) —NR3R4

wherein
R3 is
   (a) a hydrogen atom or
   (b) a C₁₋₆ alkyl group optionally having oxo group(s), and
R4 is a hydrogen atom, or
R3 and R4 in combination optionally form a 5- to 7-membered ring optionally having oxo group(s),
   (2) a C₁₋₆ alkylsulfonyl group,
   (3) a hydroxy group and
   (4) an oxo group.
   (iii) As the “C₁₋₆ alkoxy group” of the “optionally substituted C₁₋₆ alkoxy group”, for example, a C₁₋₆ alkoxy group such as methoxy, ethoxy, propoxy, isopropoxy, butoxy, isobutoxy, tert-butoxy, pentyloxy, hexyloxy and the like, and the like can be mentioned.

As the substituent, which the “C₁₋₆ alkoxy group” of the “optionally substituted C₁₋₆ alkoxy group” may have, substituents similar to the substituent, which the “C₁₋₆ alkyl group” of the above-mentioned “optionally substituted C₁₋₆ alkyl group” may have, can be mentioned.

The “optionally substituted C₁₋₆ alkoxy group” is preferably an unsubstituted C₁₋₆ alkoxy group.

R2 is a hydrogen atom, an optionally substituted C₁₋₃ alkyl group or a C₃₋₆ cycloalkyl group.

As the “C₁₋₃ alkyl group” of the “optionally substituted C₁₋₃ alkyl group”, for example, methyl, ethyl, propyl, isopropyl and the like can be mentioned.

As the substituent which the “C₁₋₃ alkyl group” of the “optionally substituted C₁₋₃ alkyl group” may have, substituents similar to the substituent which the “C₁₋₆ alkyl group” of the aforementioned “optionally substituted C₁₋₆ alkyl group” for R1′ may have, can be mentioned, and a halogen atom (e.g., fluorine, chlorine, bromine, iodine), a cycloalkyl group (e.g., cyclopropyl) and the like are preferable. Of these, a halogen atom (e.g., fluorine, chlorine, bromine, iodine) is preferable, and fluorine is particularly preferable.

As the “C₃₋₆ cycloalkyl group”, for example, cyclopropyl, cyclobutyl, cyclopentyl, cyclohexyl and the like can be mentioned.

R2 is preferably a hydrogen atom, methyl, trifluoromethyl, cyclopropylmethyl, ethyl or cyclopropyl, more preferably a hydrogen atom, methyl or trifluoromethyl.

Ring A is a piperidine ring optionally further having substituent(s). In other words, ring A may further have 1 to 8 substituents besides R1 at the 1-position, NH at the 4-position and phenyl group at the 3-position.

As the substituent which the “piperidine ring” of the “piperidine ring optionally having substituent(s)” may have, substituents similar to the substituent which the “5- or 6-membered nitrogen-containing heterocyclic group” of the “optionally substituted 5- or 6-membered nitrogen-containing heterocyclic group” for R′ or the “C₁₋₆ alkyl group” of the “optionally substituted C₁₋₆ alkyl group” for R′ may have, can be mentioned.

Ring A preferably has no substituent besides R1, NH and phenyl group.

The optically active compound (I) does not include cis-1-(methoxyacetyl)-N-[2-methoxy-5-[5-(trifluoromethyl)-1H-tetrazol-1-yl]benzyl]-3-phenyl-4-piperidinamine, and cis-1-[(1-acetyl-4-piperidinyl)carbonyl]-N-[2-methoxy-5-[5-(trifluoromethyl)-1H-tetrazol-1-yl]benzyl]-3-phenyl-4-piperidinamine.

Of the optically active compounds (I), a compound having the configuration represented by the formula (I-A) (that is, the 3-position and the 4-position on the piperidine ring are in cis configuration) is preferable.

In compound (I), a compound represented by the formula: 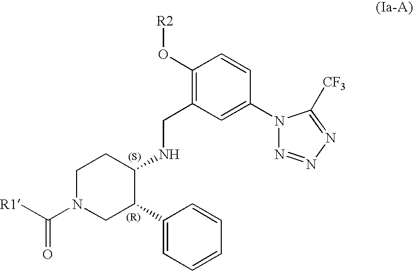
wherein R1′ is
(i) a 5- or 6-membered nitrogen-containing heterocyclic group optionally having C₁₋₆ alkylsulfonyl group(s),
(ii) a C₁₋₆ alkyl group optionally having 1 to 3 substituents selected from

(1) —NR3R4

wherein

R3 is
(a) a hydrogen atom or
(b) a C₁₋₆ alkyl group optionally having oxo group(s), and

R4 is a hydrogen atom, or

R3 and R4 in combination optionally form a 5- to 7-membered ring optionally having oxo group(s),

(2) a C₁₋₆ alkylsulfonyl group,

(3) a hydroxy group and

(4) an oxo group, or

(iii) a C₁₋₆ alkoxy group, and

R2 is a hydrogen atom, methyl or trifluoromethyl, is preferable.

Of compounds (I), N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide and a salt thereof are particularly preferable.

The compounds (I) including N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide and a salt thereof (hereinafter to be abbreviated as “compound A”) and a crystal thereof (hereinafter to be abbreviated as “the compound of the present invention”) can be produced according to the production method described in WO03/101964, which is concretely the following method. Note that the starting compound may be in the form of a salt. As such salt, for example, those similar to the salts mentioned below, and the like can be mentioned.

The compound obtained in each step can be used for the next reaction in the form of a reaction mixture or a crude product. It can also be isolated from the reaction mixture according to a conventional method and can be easily purified by separation means such as recrystallization, distillation, chromatography and the like.

When the compound in the formula is commercially available, a commercial product can also be used as it is.

Of compounds (I), a compound wherein R1 is R1′-(C═O)—(R1′ is as defined above), which is represented by the formula (Ia): 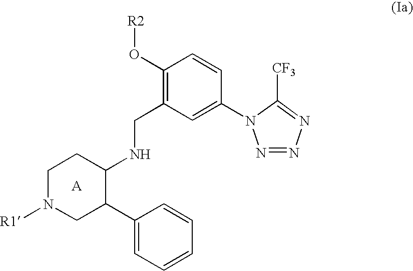
wherein each symbol is as defined above, or a salt thereof (hereinafter to be referred to as compound (Ia)) can be produced according to the following Method A or Method B.
[Method A]

Compound (Ia) can be produced by reacting a compound represented by the formula (Ib): 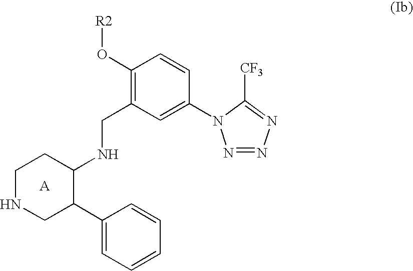
wherein each symbol is as defined above, or a salt thereof (hereinafter to be referred to as compound (Ib)) with a compound represented by the formula (II):
R1′COOH (II)
wherein R1′ is as defined above, or a salt thereof (hereinafter to be referred to as compound (II)), or a reactive derivative thereof, which is an acylating agent.

As the reactive derivative of compound (II), for example, a compound represented by the formula (IIa):
R1′-(C═O)-L (IIa)
wherein L is a leaving group and R1′ is as defined above,
or a salt thereof (hereinafter to be referred to as reactive derivative (IIa)) can be mentioned.

As the leaving group for L, for example,

a halogen atom (e.g., a chlorine atom, a bromine atom, an iodine atom),

a substituted sulfonyloxy group (e.g., a C₁₋₆ alkylsulfonyloxy group such as methanesulfonyloxy, ethanesulfonyloxy and the like; a C₆₋₁₄ arylsulfonyloxy group such as benzenesulfonyloxy, p-toluenesulfonyloxy and the like; a C₇₋₁₆ aralkylsulfonyloxy group such as benzylsulfonyloxy and the like, etc.),

an acyloxy group (acetoxy, tert-butylcarbonyloxy, benzoyloxy etc.),

an oxy group substituted by a hetero ring or an aryl group (succinimide, benzotriazole, quinoline, 4-nitrophenyl etc.),

a hetero ring (imidazole etc.)

and the like can be mentioned.

This reaction is generally carried out in a solvent, though subject to change depending on the kind of reactive derivative (IIa) and compound (Ib), and a convenient base may be added to promote the reaction.

In the reaction between compound (Ib) and reactive derivative (IIa), as the solvent, for example, hydrocarbons such as benzene, toluene and the like; ethers such as ethyl ether, dioxane, tetrahydrofuran and the like; esters such as ethyl acetate and the like; halogenated hydrocarbons such as chloroform, dichloromethane and the like; amides such as N,N-dimethylformamide and the like; aromatic amines such as pyridine and the like; water and the like can be mentioned. They may be used in a mixture at an appropriate ratio.

As the base, for example, alkali metal hydroxides such as sodium hydroxide, potassium hydroxide and the like; hydrogencarbonates such as sodium hydrogencarbonate, potassium hydrogencarbonate and the like; carbonates such as sodium carbonate, potassium carbonate and the like; acetates such as sodium acetate and the like; tertiary amines such as trimethylamine, triethylamine, N-methylmorpholine and the like; aromatic amines such as pyridine, picoline, N,N-dimethylaniline and the like, and the like can be mentioned. The amount of the base to be used is, for example, about 1 to about 100 mol, preferably about 1 to about 10 mol, per 1 mol of compound (Ib).

The amount of reactive derivative (IIa) to be used is generally about 1 to about 10 mol, preferably about 1 to about 3 mol, per 1 mol of compound (Ib).

The reaction temperature is generally about −10° C. to about 150° C., preferably about 0° C. to about 100° C., and the reaction time is generally about 15 min. to about 24 hrs, preferably about 30 min. to about 16 hrs.

When compound (II) is used as an acylating agent, for example, compound (Ia) can be produced by the use of a condensing agent. As the “condensing agent”, for example, N,N′-dicyclohexylcarbodiimide, 1-ethyl-3-(3-dimethylaminopropyl)carbodiimide hydrochloride, carbonyldiimidazole, di-(N-succinimidyl)carbonate, N-ethyl-5-phenylisoxazolium-3′-sulfonate, 1H-benzotriazol-1-yloxytris(dimethylamino)phosphonium hexafluorophosphate, O-(benzotriazol-1-yl)-N,N,N′,N′-tetramethyluronium hexafluorophosphate, an organophosphorus compound and the like can be mentioned.

The “organophosphorus compound” is reacted, for example, in the presence of a base, according to the methods described in JP-A-58-43979 and the like. As the “organophosphorus compound”, for example, alkyl o-phenylenephosphate such as methyl o-phenylenephosphate, ethyl o-phenylenephosphate (EPPA) and the like, aryl o-phenylenephosphate such as phenyl o-phenylenephosphate, p-chlorophenyl o-phenylenephosphate and the like, and the like can be mentioned, and EPPA is particularly preferable.

As the base, for example, alkylamines such as trimethylamine, triethylamine, diisopropylethylamine, tri(n-butyl)amine and the like; cyclic amines such as pyridine, 2,6-lutidine and the like, and the like can be mentioned. Of these, tertiary amines such as diisopropylethylamine and the like are preferable.

The amount of compound (II), the base and the condensing agent to be used is each generally about 1 to about 10 mol, preferably about 1 to about 5 mol, per 1 mol of compound (Ib).

As the solvent, for example, hydrocarbons such as benzene, toluene and the like; ethers such as ethyl ether, dioxane, tetrahydrofuran and the like; esters such as ethyl acetate and the like; halogenated hydrocarbons such as chloroform, dichloromethane and the like; amides such as N,N-dimethylformamide and the like; aromatic amines such as pyridine and the like, and the like can be mentioned. They may be used in a mixture at an appropriate ratio.

The reaction temperature is generally about −10° C. to about 150° C., preferably about 0° C. to about 100° C., and the reaction time is generally about 15 min. to about 24 hrs, preferably about 30 min. to about 16 hrs.

In Method A, compound (Ib) which is used as a starting compound, can be produced by subjecting compound (Ia) obtained according to the below-mentioned Method B to deacylation and the like.

The deacylation can be carried out according to a known method, for example, the methods described in Theodora W. Greene, Peter G. M. Wuts, “Protective Groups in Organic Synthesis, 3^{rd} Ed.” (1999) Wiley-Interscience and the like or an analogous method thereto. The reaction is generally carried out, though subject to change depending on the kind of compound (Ia), in the presence of an acid or a base in, where necessary, a solvent that does not adversely affect the reaction.

As the acid, mineral acids (e.g., hydrochloric acid, hydrobromic acid, sulfuric acid etc.), carboxylic acids (e.g., acetic acid, trifluoroacetic acid, trichloroacetic acid etc.), sulfonic acids (e.g., methanesulfonic acid, toluenesulfonic acid etc.), Lewis acids (e.g., aluminum chloride, tin chloride, zinc bromide etc.) and the like can be mentioned. Two or more kinds of these acids may be used in a mixture as necessary. While the amount of the acid to be used varies depending on the kinds of the solvent and other reaction conditions, it is generally about 0.1 mol or more per 1 mol of compound (Ia), and the acid can also be used as a solvent.

As the base, inorganic base (alkali metal hydroxides such as sodium hydroxide, potassium hydroxide and the like; alkali metal hydrogencarbonates such as sodium hydrogencarbonate, potassium hydrogencarbonate and the like; alkali metal carbonates such as sodium carbonate, potassium carbonate and the like; alkoxides such as sodium methoxide, sodium ethoxide and the like, etc.) and organic base (amines such as trimethylamine, triethylamine, diisopropylethylamine and the like; cyclic amines such as pyridine, 4-dimethylaminopyridine and the like, etc.) and the like can be mentioned. Of these, sodium hydroxide, potassium hydroxide, sodium ethoxide and the like are preferable. While the amount of the base to be used varies depending on the kind of the solvent and other reaction conditions, it is generally about 0.1 to about 10 mol, preferably about 0.1 to about 5 mol, per 1 mol of compound (Ia).

As the solvent that does not adversely affect the reaction, for example, alcohols such as methanol, ethanol, propanol, 2-propanol, butanol, isobutanol, tert-butanol and the like; aromatic hydrocarbons such as benzene, toluene, xylene and the like; aliphatic hydrocarbons such as hexane, heptane and the like; halogenated hydrocarbons such as dichloromethane, chloroform and the like; ethers such as diethyl ether, diisopropyl ether, tert-butyl methyl ether, tetrahydrofuran, dioxane, dimethoxyethane and the like; nitriles such as acetonitrile and the like; esters such as ethyl acetate and the like; carboxylic acids such as acetic acid and the like; amides such as N,N-dimethylformamide, N,N-dimethylacetamide and the like; sulfoxides such as dimethyl sulfoxide and the like; water and the like can be mentioned. Two or more kinds of these solvents may be used in a mixture at an appropriate ratio.

The reaction temperature is, for example, within the range of about −50° C. to about 200° C., preferably within the range of about 0° C. to about 100° C., and the reaction time varies depending on the kind of compound (Ia), the reaction temperature and the like, and it is, for example, about 0.5 to about 100 hrs, preferably about 0.5 to about 24 hrs.
[Method B] 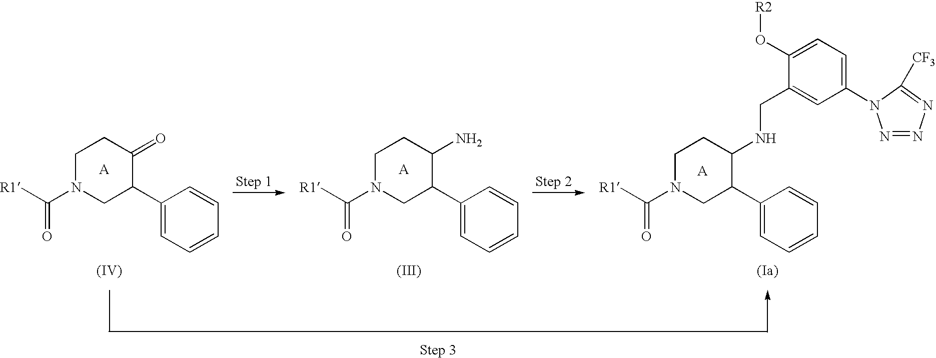
wherein each symbol is as defined above.

Compound (IV) to be used as a starting compound in this method can be produced according to the production method described in WO03/101964, and the like.

(Step 1)

In this step, a compound represented by the formula (IV) (hereinafter to be referred to as compound (IV)) is converted to imine or oxime, and the imine or oxime is subjected to reduction to give a compound represented by the formula (III) (hereinafter to be referred to as amine compound (III)).

The conversion of compound (IV) to the imine or oxime can be carried out according to a known method by, for example, using various amines in a solvent inert to the reaction.

As the amines, ammonia such as aqueous ammonia, ammonium chloride, ammonium acetate and the like; hydroxylamines such as hydroxylamine, O-methylhydroxylamine, O-benzylhydroxylamine and the like; organic amines such as benzylamine, aminodiphenylmethane, 1-phenylethylamine and the like, and the like can be mentioned, and these may be used in the form of a salt such as hydrochloride, sulfate and the like, or in the form of an aqueous solution thereof. The amount of the amines to be used is, for example, about 1 to about 50 mol, preferably about 1 to about 10 mol, per 1 mol of compound (IV).

As the solvent inert to the reaction, for example, aromatic hydrocarbons such as toluene, xylene and the like; aliphatic hydrocarbons such as heptane, hexane and the like; halogenated hydrocarbons such as chloroform, dichloromethane and the like; ethers such as diethyl ether, tetrahydrofuran, dioxane and the like; alcohols such as methanol, ethanol, 2-propanol, butanol, benzyl alcohol and the like; nitriles such as acetonitrile and the like; N,N-dimethylformamide; dimethyl sulfoxide and the like can be mentioned. These solvents may be used in a mixture at an appropriate ratio.

The reaction can be advantageously carried out by the addition of a catalyst as necessary. As such catalyst, mineral acids (e.g., hydrochloric acid, hydrobromic acid, sulfuric acid etc.), carboxylic acids (e.g., formic acid, acetic acid, propionic acid, trifluoroacetic acid etc.), sulfonic acids (e.g., methanesulfonic acid, p-toluenesulfonic acid etc.), Lewis acids (e.g., aluminum chloride, zinc chloride, zinc bromide, boron trifluoride, titanium chloride etc.), acetate (e.g., sodium acetate, potassium acetate etc.), molecular sieves (e.g., molecular sieves 3A, 4A, 5A etc.), dehydrating agent (e.g., magnesium sulfate etc.) and the like can be mentioned. The amount of the catalyst to be used is, for example, about 0.01 to about 50 mol, preferably about 0.1 to about 10 mol, per 1 mol of compound (IV).

The reaction temperature is generally about 0° C. to about 200° C., preferably about 20° C. to about 150° C., and the reaction time is generally about 0.5 hr to about 48 hrs, preferably about 0.5 hr to about 24 hrs.

The conversion of the imine or oxime to the amine compound (III) can be carried out by various reductions in a solvent inert to the reaction. The reduction can be carried out according to a method known per se, such as a method using a metal hydride and a method including catalytic hydrogenation.

As the metal hydride, for example, sodium borohydride, lithium borohydride, zinc borohydride, sodium cyanoborohydride, sodium triacetoxyborohydride, lithium cyanoborohydride, diisobutylaluminum hydride, aluminum hydride, lithium aluminum hydride, borane complex (borane-THF complex etc.), catechol borane and the like can be mentioned, and sodium borohydride, sodium cyanoborohydride, sodium triacetoxyborohydride and the like are preferable. The amount of the metal hydride to be used is, for example, about 1 to about 50 mol, preferably about 1 to about 10 mol, per 1 mol of the imine or oxime.

The reduction using a metal hydride is generally carried out in a solvent inert to the reaction. As such solvent, for example, aromatic hydrocarbons such as toluene, xylene and the like; aliphatic hydrocarbons such as heptane, hexane and the like; halogenated hydrocarbons such as chloroform, dichloromethane and the like; ethers such as diethyl ether, tetrahydrofuran, dioxane and the like; alcohols such as methanol, ethanol, 2-propanol, butanol, benzyl alcohol and the like; nitriles such as acetonitrile and the like; N,N-dimethylformamide; dimethyl sulfoxide and the like can be mentioned. These solvents may be used in a mixture at an appropriate ratio.

The reaction temperature is generally about −80° C. to about 80° C., preferably about −40° C. to about 40° C., and the reaction time is generally about 5 min. to about 48 hrs, preferably about 1 hr to about 24 hrs.

The catalytic hydrogenation can be carried out in the presence of a catalyst under a hydrogen atmosphere. As the catalyst, palladium catalysts such as palladium carbon, palladium hydroxide carbon, palladium oxide and the like; nickel catalysts such as Raney nickel catalyst and the like; platinum catalysts such as platinum oxide, platinum carbon and the like; rhodium catalysts such as rhodium carbon and the like; and the like can be mentioned. Its amount of use is about 0.001 to about 1 mol, preferably about 0.01 to about 0.5 mol, per 1 mol of the imine or oxime.

The catalytic hydrogenation is generally carried out in a solvent inert to the reaction. As such solvent, for example, alcohols such as methanol, ethanol, propanol, butanol and the like; hydrocarbons such as benzene, toluene, xylene and the like; halogenated hydrocarbons such as dichloromethane, chloroform and the like; ethers such as diethyl ether, dioxane, tetrahydrofuran and the like; esters such as ethyl acetate and the like; amides such as N,N-dimethylformamide and the like; carboxylic acids such as acetic acid and the like; water and a mixture thereof can be mentioned.

The hydrogen pressure at which the reaction is carried out is generally about 1 to about 50 atm, preferably about 1 to about 10 atm. The reaction temperature is generally about 0° C. to about 150° C., preferably about 20° C. to about 100° C., and the reaction time is generally about 5 min. to about 72 hrs, preferably about 0.5 hr to about 40 hrs.

In this Step, the next reduction is carried out without isolating the imine or oxime, which is an intermediate, to directly give amine compound (III) from compound (IV). In this case, the pH of the reaction mixture is preferably about 4 to about 5.

(Step 2)

In this step, amine compound (III) is converted to compound (Ia) by subjecting amine compound (III) to alkylation or reductive alkylation.

The alkylation can be carried out according to a method known per se. For example, amine compound (III) is reacted with a compound represented by the formula (V): 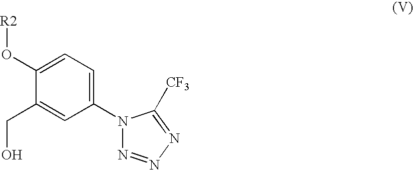
wherein the symbol in the formula is as defined above, or a salt thereof (hereinafter to be referred to as compound (V)) or a reactive derivative thereof, which is an alkylating agent.

As the reactive derivative of compound (V), for example, a compound represented by the formula (Va): 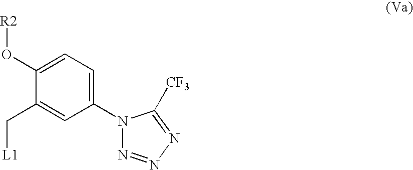
wherein L1 is a leaving group and R2 is as defined above, or a salt thereof (hereinafter to be referred to as reactive derivative (Va)) can be mentioned.

As the leaving group for L1, for example,

a halogen atom (e.g., a chlorine atom, a bromine atom, an iodine atom),

a substituted sulfonyloxy group (e.g., a C₁₋₆ alkylsulfonyloxy group such as methanesulfonyloxy, ethanesulfonyloxy and the like; a C₆₋₁₄ arylsulfonyloxy group such as benzenesulfonyloxy, p-toluenesulfonyloxy and the like; a C₇₋₁₆ aralkylsulfonyloxy group such as benzylsulfonyloxy and the like; a C₁₋₆ alkoxysulfonyloxy group such as methoxysulfonyloxy and the like, etc.)

and the like can be mentioned.

The reaction using compound (V) or reactive derivative (Va) as an alkylating agent can be generally carried out by, though subject to change depending on the kind of compound (V) or reactive derivative (Va) or amine compound (III), reacting compound (V) or reactive derivative (Va) with amine compound (III) in a solvent in the presence of a base.

As the solvent, for example, alcohols such as methanol, ethanol, propanol and the like; ethers such as dimethoxyethane, dioxane, tetrahydrofuran and the like; ketones such as acetone and the like; nitrites such as acetonitrile and the like; amides such as N,N-dimethylformamide and the like; sulfoxides such as dimethyl sulfoxide and the like; water and the like can be mentioned. These solvents may be used in a mixture at an appropriate ratio.

As the base, for example, organic bases such as trimethylamine, triethylamine, N-methylmorpholine, pyridine, picoline, N,N-dimethylaniline and the like; and inorganic bases such as potassium carbonate, sodium carbonate, potassium hydroxide, sodium hydroxide and the like, can be mentioned. The amount of the base to be used is, for example, about 1 to about 100 mol, preferably about 1 to about 10 mol, per 1 mol of amine compound (III).

As reactive derivative (Va), for example, halides (e.g., chloride, bromide, iodide etc.), sulfates, sulfonates (e.g., methanesulfonate, p-toluenesulfonate, benzenesulfonate etc.) and the like can be mentioned, and halides are particularly preferably used. The amount of compound (V) or reactive derivative (Va) to be used is, for example, about 1 to about 5 mol, preferably about 1 to about 3 mol, per 1 mol of amine compound (III).

Where necessary, the reaction can be promoted by adding an additive. As such additive, for example, iodides such as sodium iodide, potassium iodide and the like can be mentioned. Its amount of use is about 0.1 to about 10 mol, preferably about 0.1 to about 5 mol, per 1 mol of amine compound (III).

The reaction temperature is generally about −10° C. to about 200° C., preferably about 0° C. to about 110° C., and the reaction time is generally about 0.5 hr to about 48 hrs, preferably about 0.5 hr to about 16 hrs.

The reductive alkylation can be carried out according to a method known per se. For example, amine compound (III) is reacted with a compound represented by the formula (VI): 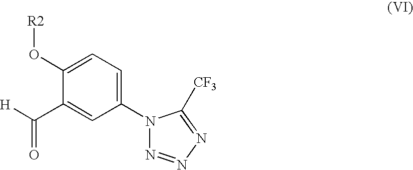
wherein the symbol in the formula is as defined above,
or a salt thereof (hereinafter to be referred to as compound (VI)), and the resulting imine or iminium ion is subjected to reduction.

The production of the imine or iminium ion and the reduction thereof can be carried-out according to the methods described in Step 1.

In this Step, the imine or iminium ion, which is an intermediate, can be subjected to the next reduction without isolation to give compound (Ia) directly from amine compound (III). In this case, the pH of the reaction mixture is preferably about 4 to about 5.

(Step 3)

In this Step, compound (IV) is converted to compound (Ia) by subjecting compound (IV) to reductive amination. This reaction can be carried out according to a method known per se. For example, compound (IV) is reacted with a compound represented by the formula (VII): 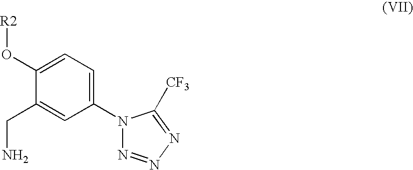
wherein the symbol in the formula is as defined above, or a salt thereof (hereinafter to be referred to as compound (VII)), and the resulting imine or iminium ion is subjected to reduction.

The production of the imine or iminium ion and reduction thereof can be carried out according to the methods described in Step 1.

In this Step, the imine or iminium ion, which is an intermediate, can be subjected to the next reduction without isolation to give compound (Ia) directly from compound (IV). In this case, the pH of the reaction mixture is preferably about 4 to about 5.

Compound (Ia) obtained by the method described in the above-mentioned Method A or Method B can be further converted to its derivatives by subjecting compound (Ia) to various known reactions such as condensation (e.g., acylation, alkylation etc.), oxidization, reduction and the like. Such reactions can be carried out according to methods known per se.

It is also possible to produce an optically active compound (I) by reacting, according to the above-mentioned Method A or Method B, an optically active compound obtained by optical resolution of the racemate of compound (Ib) or amine compound (III) according to a method known per se. As such optical resolution, for example, the below-mentioned fractional recrystallization method, chiral column method, diastereomer method and the like can be mentioned.

Of the optically active compounds (I), particularly a compound represented by the formula (I′): 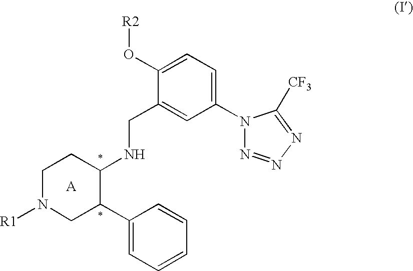
wherein each symbol is as defined above, and the amino group and the phenyl group on the piperidine are in cis configuration, or a salt thereof (hereinafter to be referred to as compound (I′)), can be produced by reacting, according to Method B, an optically active compound represented by the formula (IIIa): 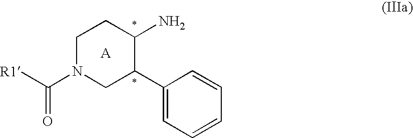
wherein each symbol is as defined above, and the amino group and the phenyl group on the piperidine are in cis configuration, or a salt thereof (hereinafter to be referred to as compound (IIIa)) in Step 2 of the above-mentioned Method B. The optically active compound (IIIa), which is used as a starting compound, can be produced according to the following Method C.
[Method C] 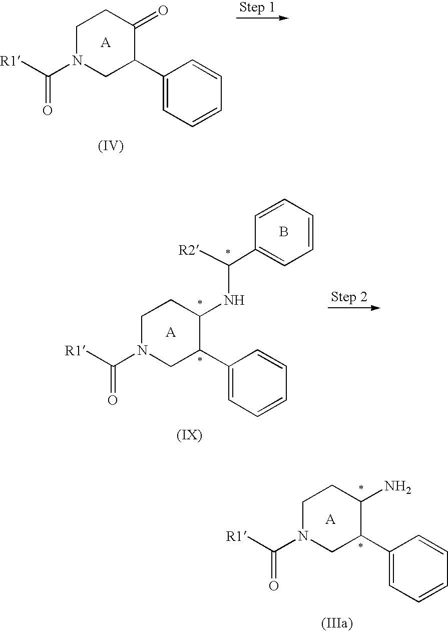
wherein R2′ is a hydrocarbon group optionally having substituent(s), ring B is an optionally fused benzene ring optionally having substituent(s), and the other symbols are as defined above.

As the “hydrocarbon group” of the “hydrocarbon group optionally having substituent(s)” for R2′, for example, a lower alkyl group (e.g., a C₁₋₆ alkyl group such as methyl, ethyl, propyl, isopropyl, butyl, isobutyl, sec-butyl, tert-butyl, pentyl, hexyl and the like, etc.), a cycloalkyl group (e.g., a C₃₋₆ cycloalkyl group such as cyclopropyl, cyclobutyl, cyclopentyl, cyclohexyl and the like, etc.), a lower alkynyl group (e.g., a C₂₋₆ alkynyl group such as ethynyl, 1-propynyl, propargyl and the like, etc.), a lower alkenyl group (e.g., a C₂₋₆ alkenyl group such as vinyl, allyl, isopropenyl, butenyl, isobutenyl and the like, etc.), an aralkyl group (e.g., a C₇₋₁₁ aralkyl group such as benzyl, α-methylbenzyl, phenethyl and the like, etc.), an aryl group (e.g., a C₆₋₁₀ aryl group such as phenyl, naphthyl and the like, etc., preferably phenyl group etc.) and the like can be mentioned.

As the substituent, which the “hydrocarbon group” of the “hydrocarbon group optionally having substituent(s)” may have, those similar to the substituents, which the “C₁₋₆ alkyl group” of the above-mentioned “optionally substituted C₁₋₆ alkyl group” may have, can be mentioned.

The “hydrocarbon group optionally having substituent(s)” for R2′ is preferably a C₁₋₃ alkyl group or a C₃₋₆ cycloalkyl group.

As the substituent which the “benzene ring” of the “optionally fused benzene ring optionally having substituent(s)” for B ring may have,

(i) an optionally halogenated C₁₋₆ alkyl group (e.g., trifluoromethyl),

(ii) a C₁₋₆ alkoxy group (e.g., methoxy),

(iii) a 5- or 6-membered aromatic heterocyclic group containing, besides carbon atom(s), 1 to 4 hetero atoms selected from a nitrogen atom, an oxygen atom and a sulfur atom (e.g., tetrazole), which is optionally substituted by an optionally halogenated C₁₋₆ alkyl group (e.g., trifluoromethyl), and the like can be mentioned. The benzene ring may be fused with a ring constituting the above-mentioned “aromatic heterocyclic group” or a benzene ring.

(Step 1)

In this Step, compound (IV) and an optically active amine represented by the formula (VIII): 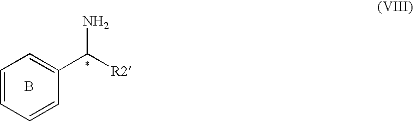
wherein each symbol is as defined above,
or a salt thereof (hereinafter to be referred to as optically active amine (VIII)) are condensed to give imine, which is then hydrogenated to be converted to a compound represented by the formula (IX), wherein the amino group and the phenyl group are in cis configuration, or a salt thereof (hereinafter to be referred to as compound (IX).

The Step to convert compound (IV) to the imine by reacting compound (IV) with optically active amine (VIII) can be carried out by a method known per se. For example, the reaction can be carried out using optically active amine (VIII) in a solvent inert to the reaction using a catalyst as necessary.

As optically active amine (VIII) to be used in this reaction, for example, (R)- or (S)-1-phenylethylamine, (R)- or (S)-1-phenylpropylamine, (R)- or (S)-1-(1-naphthyl)ethylamine, (R)- or (S)-1-(2-naphthyl)ethylamine, (R)- or (S)-1-(4-toluyl)ethylamine and the like can be mentioned. Particularly, (R)- or (S)-1-phenylethylamine is preferable.

The amount of optically active amine (VIII) to be used is about 0.9 to about 10 mol, preferably about 1 to about 2 mol, per 1 mol of compound (IV).

The solvent to be used in this reaction is not particularly limited as long as it does not adversely affect the reaction and can dissolve the starting compound and, for example, aromatic hydrocarbons such as toluene, xylene and the like; aliphatic hydrocarbons such as heptane, hexane and the like; halogenated hydrocarbons such as chloroform, dichloromethane and the like; ethers such as diethyl ether, tetrahydrofuran, dioxane and the like; alcohols such as methanol, ethanol, 2-propanol, butanol, benzyl alcohol and the like; nitriles such as acetonitrile and the like; N,N-dimethylformamide; dimethyl sulfoxide and the like can be mentioned. These solvents may be used in a mixture at an appropriate ratio. Particularly, toluene is preferable. The amount of the solvent to be used is appropriately determined according to the solubility of compound (IV) and an optically active amine (VIII), and the like. The reaction can be carried out in the state almost free of a solvent or using a solvent in an amount not more than 100-fold weight of compound (IV). Generally, use of a solvent in a 5- to 30-fold weight of compound (IV) is preferable.

The reaction can be advantageously carried out by adding a catalyst as necessary. As such catalyst, mineral acids (e.g., hydrochloric acid, hydrobromic acid, sulfuric acid etc.), carboxylic acids (e.g., formic acid, acetic acid, propionic acid, trifluoroacetic acid etc.), sulfonic acids (e.g., methanesulfonic acid, p-toluenesulfonic acid etc.), Lewis acids (e.g., aluminum chloride, zinc chloride, zinc bromide, boron trifluoride, titanium chloride etc.), acetates (e.g., sodium acetate, potassium acetate etc.) and molecular sieves (e.g., molecular sieves 3A, 4A, 5A etc.) can be mentioned. Preferred is Lewis acid, and particularly preferred is aluminum chloride. The amount of the catalyst to be used is, for example, about 0.01 to about 10 mol, preferably about 0.02 to about 1 mol, per 1 mol of compound (IV).

While the reaction temperature varies depending on the solvent to be used, it is generally about 30° C. to about 200° C., preferably about 50° C. to about 150° C., and the reaction time is generally about 0.1 hr to about 48 hrs, preferably about 0.1 hr to about 24 hrs.

This reaction can also be promoted by azeotropic dehydration known per se.

The Step to convert to an optically active compound (IX) by hydrogenation of the imine can be carried out by a method known per se. For example, a method using metal hydride in a solvent inert to the reaction and a method involving catalytic hydrogenation in a solvent inert to the reaction can be mentioned.

As the metal hydride, for example, sodium borohydride, lithium borohydride, zinc borohydride, sodium cyanoborohydride, sodium triacetoxyborohydride, lithium cyanoborohydride, diisobutylaluminum hydride, aluminum hydride, lithium aluminum hydride, borane complex (borane-THF complex etc.), catechol borane and the like can be mentioned. Of these, sodium borohydride, sodium cyanoborohydride, sodium triacetoxyborohydride and the like are preferable. The amount of the metal hydride to be used is, for example, about 1 to about 50 mol, preferably about 1 to about 10 mol, per 1 mol of the imine.

The solvent used here is not particularly limited as long as it does not adversely affect the reaction and can dissolve the starting compound and, for example, aromatic hydrocarbons such as toluene, xylene and the like; aliphatic hydrocarbons such as heptane, hexane and the like; halogenated hydrocarbons such as chloroform, dichloromethane and the like; ethers such as diethyl ether, tetrahydrofuran, dioxane and the like; alcohols such as methanol, ethanol, 2-propanol, butanol, benzyl alcohol and the like; nitriles such as acetonitrile and the like; N,N-dimethylformamide; dimethyl sulfoxide and the like can be mentioned. These solvents may be used in a mixture at an appropriate ratio. As regards the amount of the solvent to be used, the reaction can be carried out in the state almost free of a solvent or using a solvent in an amount of about not more than 100-fold weight of the imine. Generally, use of a solvent in a 5- to 30-fold weight of the imine is preferable.

The reaction temperature is generally about −80° C. to about 200° C., preferably about −50° C. to about 100° C., and the reaction time is generally about 5 min. to about 72 hrs, preferably about 0.5 hr to about 12 hrs.

The catalytic hydrogenation can be carried out under a hydrogen atmosphere in the presence of a catalyst. As the catalyst, palladium catalysts such as palladium carbon, palladium hydroxide carbon, palladium oxide and the like; nickel catalysts such as Raney nickel catalyst and the like; platinum catalysts such as platinum oxide, platinum carbon and the like; rhodium catalysts such as rhodium carbon and the like and the like can be mentioned. Of these catalysts, a heterogeneous catalyst using nickel is preferable, and Raney nickel catalyst is particularly preferable. Its amount of use based on nickel is about 0.1 to about 200 mol, preferably about 1 to about 100 mol, per 1 mol of the imine.

The catalytic hydrogenation is generally carried out in a solvent inert to the reaction. As such solvent, for example, alcohols such as methanol, ethanol, propanol, butanol, benzyl alcohol and the like; aliphatic hydrocarbons such as heptane, hexane and the like; aromatic hydrocarbons such as benzene, toluene, xylene and the like; halogenated hydrocarbons such as dichloromethane, chloroform and the like; ethers such as diethyl ether, dimethoxyethane, dioxane, tetrahydrofuran and the like; esters such as ethyl acetate and the like; amides such as N,N-dimethylformamide and the like; carboxylic acids such as acetic acid and the like; water and a mixture thereof can be mentioned. Preferable solvent is alcohol and, ethanol is particularly preferable. As regards the amount of the solvent to be used, the reaction can be carried out in the state almost free of a solvent or using a solvent in an amount of about not more than 100-fold weight of the imine. Generally, use of a solvent in a 5- to 30-fold weight of the imine is preferable.

The hydrogenation can be carried out by any of a batch type reaction and a continuous reaction. The hydrogen pressure at which the reaction is carried out is generally about 0.1 to about 5 MPa, and preferably about 0.1 to about 1 MPa. The reaction temperature is generally about 0° C. to about 150° C., preferably about 20° C. to about 50° C., and the reaction time is generally about 5 min. to about 120 hrs.

Of optically active amines (VIII), a desired optically active form of compound (IX) can be selectively obtained by appropriately selecting an (R)-configuration or an (S)-configuration.

(Step 2)

In this Step, compound (IX) obtained in Step 1 is subjected to hydrogenolysis to give compound (IIIa) wherein the amino group and the phenyl group are in cis configuration. The hydrogenolysis can be carried out according to a method known per se and, for example, a method including catalytic hydrogenation can be mentioned.

The catalytic hydrogenation can be carried out under a hydrogen atmosphere in the presence of a catalyst. As the catalyst to be used, for example, palladium catalysts such as palladium carbon, palladium hydroxide carbon, palladium oxide and the like; nickel catalysts such as Raney nickel and the like; platinum catalysts such as platinum oxide, platinum carbon and the like; rhodium catalysts such as rhodium carbon and the like, and the like can be mentioned. Of these catalysts, a heterogeneous catalyst supporting palladium is preferable, and palladium carbon and palladium hydroxide carbon are particularly preferable. Its amount of use based on palladium is about 0.0001 to about 1 mol, preferably about 0.001 to about 0.5 mol, per 1 mol of compound (IX).

The catalytic hydrogenation is generally carried out in a solvent inert to the reaction. As such solvent, for example, alcohols such as methanol, ethanol, propanol, butanol, benzyl alcohol and the like; aliphatic hydrocarbons such as heptane, hexane and the like; aromatic hydrocarbons such as benzene, toluene, xylene and the like; halogenated hydrocarbons such as dichloromethane, chloroform and the like; ethers such as diethyl ether, dimethoxyethane, dioxane, tetrahydrofuran and the like; esters such as ethyl acetate and the like; nitrites such as acetonitrile and the like; amides such as N,N-dimethylformamide and the like; carboxylic acids such as acetic acid and the like; water and a mixture thereof can be mentioned. Preferable solvent is alcohol and ethanol is particularly preferable. As regards the amount of the solvent to be used, the reaction can be carried out in the state almost free of a solvent or using a solvent in an amount of about not more than 100-fold weight of compound (IX). Generally, use of a solvent in a 5- to 30-fold weight of compound (IX) is preferable.

The hydrogenation can be carried out by any of a batch type reaction and a continuous reaction. The hydrogen pressure at which the reaction is carried out is, for example, generally about 0.1 to about 5 MPa, preferably about 0.1 to about 1 MPa. The reaction temperature is generally about 0° C. to about 200° C., preferably about 20° C. to about 60° C., and the reaction time is generally about 5 min. to about 120 hrs.

In this method, the next hydrogenolysis is carried out to directly give compound (IIIa) from compound (IV), without isolating compound (IX), which is an intermediate.

An optically active compound A of the present invention represented by the formula: 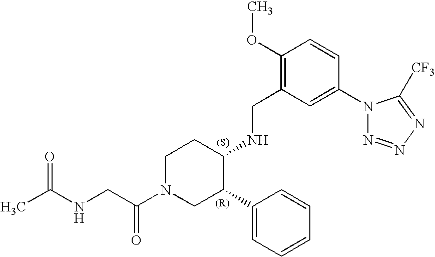
can be produced by the aforementioned Method A or Method B. It is preferable to produce the compound using, as a starting amine compound, optically active N-{2-[(3R,4S)-4-amino-3-phenylpiperidin-1-yl]-2-oxoethyl}acetamide represented by the formula: 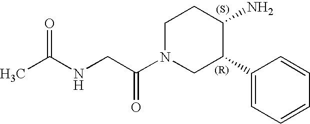
or a salt thereof, by subjecting the compound to alkylation or reductive alkylation according to the aforementioned Method B, Step 2. The reductive alkylation is more preferable, wherein N-{2-[(3R,4S)-4-amino-3-phenylpiperidin-1-yl]-2-oxoethyl}acetamide or a salt thereof and 2-methoxy-5-[5-(trifluoromethyl)-1H-tetrazol-1-yl]benzaldehyde represented by the formula: 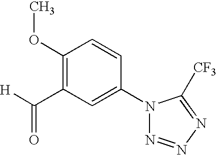
or a salt thereof are reacted, and the resulting imine or iminium ion is subjected to reduction. Production of the imine or the iminium ion and reduction thereof can be carried out according to the method described in Method B. Step 2.

The production of imine or iminium ion is generally performed in a solvent that does not adversely affect the reaction. As such solvent, for example, aromatic hydrocarbons such as toluene, xylene and the like; aliphatic hydrocarbons such as heptane, hexane and the like; halogenated hydrocarbons such as chloroform, dichloromethane and the like; ethers such as diethyl ether, tetrahydrofuran, dioxane and the like; esters such as ethyl acetate and the like; carboxylic acids such as acetic acid and the like; alcohols such as methanol, ethanol, 2-propanol, butanol, benzyl alcohol and the like; nitrites such as acetonitrile and the like; amides such as N,N-dimethylformamide, N,N-dimethylacetamide and the like; dimethyl sulfoxide and the like can be mentioned. These solvents may be used in a mixture at an appropriate ratio.

The reaction can be advantageously carried out by adding a catalyst as necessary. As such catalyst, mineral acids (e.g., hydrochloric acid, hydrobromic acid, sulfuric acid etc.), sulfonic acids (e.g., methanesulfonic acid, p-toluenesulfonic acid etc.), Lewis acids (e.g., aluminum chloride, zinc chloride, zinc bromide, boron trifluoride, titanium chloride etc.), acetate (e.g., sodium acetate, potassium acetate etc.), molecular sieves (e.g., molecular sieves 3A, 4A, 5A etc.) can be mentioned. The amount of the catalyst to be used is, for example, 0 to about 50 mol, preferably 0 to about 10 mol, per 1 mol of N-{2-[(3R,4S)-4-amino-3-phenylpiperidin-1-yl]-2-oxoethyl}acetamide or a salt thereof.

The reaction temperature is generally about 0° C. to about 200° C., preferably about 20° C. to about 150° C., and the reaction time is generally about 0.5 hr to about 48 hrs, preferably about 0.5 hr to about 24 hrs.

While the imine produced here can be isolated and purified, for example, by conventional separation means such as recrystallization, distillation, chromatography and the like, it is preferable to carry out reduction without isolation.

Imine or iminium ion can be reduced, for example, by a method using metal hydride or a method involving catalytic hydrogenation.

As the metal hydride, metal hydrides exemplified in Method B, Step 1 can be mentioned. Preferred are sodium borohydride, sodium cyanoborohydride, sodium triacetoxyborohydride and the like, and most preferred is sodium triacetoxyborohydride. The amount of the reducing agent to be used is, for example, about 1 to about 50 mol, preferably about 1 to about 10 mol, per 1 mol of imine or iminium ion.

As the reaction solvent, for example, aromatic hydrocarbons such as toluene, xylene and the like; aliphatic hydrocarbons such as heptane, hexane and the like; halogenated hydrocarbons such as chloroform, dichloromethane and the like; ethers such as diethyl ether, tetrahydrofuran, dioxane and the like; esters such as ethyl acetate and the like; carboxylic acids such as acetic acid and the like; alcohols such as methanol, ethanol, 2-propanol, butanol, benzyl alcohol and the like; nitrites such as acetonitrile and the like; amides such as N,N-dimethylformamide and the like; dimethyl sulfoxide and the like can be mentioned. These solvents may be used in a mixture at an appropriate ratio. Preferable solvents are carboxylic acids, halogenated hydrocarbons and esters. More preferable solvents are a mixed solvent of carboxylic acids and halogenated hydrocarbons and a mixed solvent of carboxylic acids and esters. Particularly, preferable carboxylic acids include acetic acid, preferable esters include ethyl acetate and preferable halogenated hydrocarbons include dichloromethane. Especially preferred are a mixed solvent of dichloromethane and acetic acid and a mixed solvent of ethyl acetate and acetic acid.

The reaction can be advantageously carried out by adding an additive as necessary. As the additive, organic amines (e.g., alkylamines such as trimethylamine, triethylamine, diisopropylethylamine, N-methylmorpholine and the like, aromatic amines such as pyridine, N,N-dimethylaniline and the like, etc.) is preferable. Of these, triethylamine and diisopropylethylamine are preferable. Its amount of use is, for example, about 0.001 to about 10 mol, preferably about 0.01 to about 5 mol, per 1 mol of imine or iminium ion.

The reaction temperature is generally about −80° C. to about 80° C., preferably about −40° C. to about 40° C., and the reaction time is generally about 5 min. to about 48 hrs, preferably about 1 hr to about 24 hrs.

The catalytic hydrogenation can be carried out under a hydrogen atmosphere in the presence of a catalyst. As the catalyst to be used, the catalysts exemplified in Method B, Step 1 can be mentioned. Preferred are palladiums such as palladium carbon, palladium hydroxide carbon, palladium oxide and the like, and most preferred is palladium carbon. The amount of the catalyst to be used based on palladium is about 0.001 to about 1 mol, preferably about 0.01 to about 0.5 mol, per 1 mol of imine or iminium ion.

The catalytic hydrogenation is generally carried out in a solvent inert to the reaction. As such solvent, for example, alcohols such as methanol, ethanol, propanol, butanol and the like; hydrocarbons such as benzene, toluene, xylene and the like; halogenated hydrocarbons such as dichloromethane, chloroform and the like; ethers such as diethyl ether, dioxane, tetrahydrofuran and the like; esters such as ethyl acetate and the like; amides such as N,N-dimethylformamide, N,N-dimethylacetamide and the like; carboxylic acids such as acetic acid and the like; water and the like can be mentioned. These solvents may be used in a mixture at an appropriate ratio. Preferable solvents are amides and esters. More preferable solvent is a mixed solvent of amides and esters. Particularly, preferable amides include N,N-dimethylacetamide and preferable esters include ethyl acetate. Most preferred is a mixed solvent of N,N-dimethylacetamide and ethyl acetate.

The reaction can be advantageously carried out by adding an additive as necessary. As the additive, organic amines (e.g., alkylamines such as trimethylamine, triethylamine, diisopropylethylamine, N-methylmorpholine and the like, aromatic amines such as pyridine, N,N-dimethylaniline and the like, etc.) is preferable. Of these, triethylamine and diisopropylethylamine are preferable. Its amount of use is about 0.001 to 10 mol, preferably about 0.01 to 5 mol, per 1 mol of imine or iminium ion.

The hydrogen pressure at which the reaction is carried out is generally about 1 to about 50 atm, and preferably about 1 to about 10 atm. The reaction temperature is generally about 0° C. to about 150° C., preferably about 20° C. to about 100° C., and the reaction time is generally about 5 min. to about 72 hrs, preferably about 0.5 hr to about 40 hrs.

For reduction of imine and iminium ion, catalytic hydrogenation is more preferable.

N-{2-[(3R,4S)-4-amino-3-phenylpiperidin-1-yl]-2-oxoethyl}acetamide or a salt thereof, which is an optically active amine compound to be used as a starting compound, can be produced by Method D shown below.
[Method D] 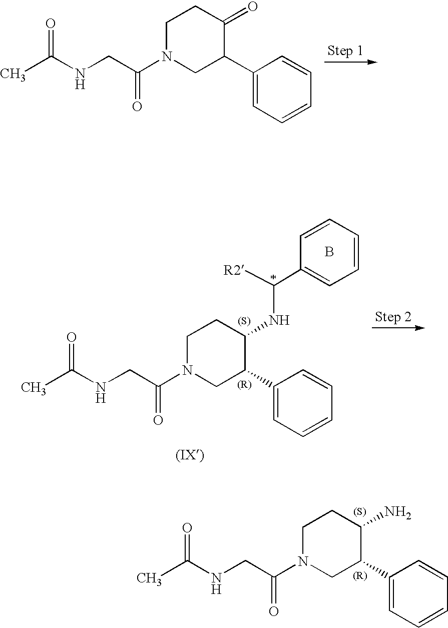
wherein each symbol is as defined above.
(Step 1)

This step can be performed according to the method described in Method C, Step 1. In this step, N-[2-oxo-2-(4-oxo-3-phenylpiperidin-1-yl)ethyl]acetamide and optically active amine (VIII): 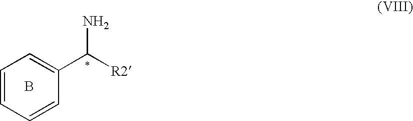
wherein each symbol is as defined above, are condensed to convert to imine, and the imine is hydrogenated to convert a compound represented by the formula (IX′) (hereinafter to be referred to as compound (IX′)).

The step wherein N-[2-oxo-2-(4-oxo-3-phenylpiperidin-1-yl)ethyl]acetamide is reacted with optically active amine (VIII) to convert it to imine can be carried out according to a method known per se and, for example, using optically active amine (VIII) in a solvent inert to the reaction and using a catalyst as necessary.

As the optically active amine (VIII) to be used in this reaction, an optical isomer having (S)-configuration is preferable and, for example, (S)-1-phenylethylamine, (S)-1-phenylpropylamine, (S)-1-(1-naphthyl)ethylamine, (S)-1-(2-naphthyl)ethylamine, (S)-1-(4-toluyl)ethylamine and the like can be mentioned. Of these, a compound wherein R2′ is a methyl group is preferable, and (S)-1-phenylethylamine is particularly preferable. The amount of the optically active amine (VIII) to be used is about 0.9 to about 10 mol, preferably about 1 to about 2 mol, per 1 mol of N-[2-oxo-2-(4-oxo-3-phenylpiperidin-1-yl)ethyl]acetamide.

The solvent to be used in this reaction is not particularly limited as long as it does not adversely affect the reaction and can dissolve the starting compound and, for example, aromatic hydrocarbons such as toluene, xylene and the like; aliphatic hydrocarbons such as heptane, hexane and the like; halogenated hydrocarbons such as chloroform, dichloromethane and the like; ethers such as diethyl ether, tetrahydrofuran, dioxane and the like; alcohols such as methanol, ethanol, 2-propanol, butanol, benzyl alcohol and the like; nitriles such as acetonitrile and the like; N,N-dimethylformamide; dimethyl sulfoxide and the like can be mentioned. These solvents may be used in a mixture at an appropriate ratio. Particularly, toluene is preferable. The amount of the solvent to be used is appropriately determined based on the solubility of N-[2-oxo-2-(4-oxo-3-phenylpiperidin-1-yl)ethyl]acetamide and optically active amine (VIII), and the like. The reaction can be carried out in the state almost free of a solvent or using a solvent in an amount not more than 100-fold weight of N-[2-oxo-2-(4-oxo-3-phenylpiperidin-1-yl)ethyl]acetamide. Generally, use of a solvent in a 5- to 30-fold weight is preferable.

The reaction can be advantageously carried out by adding a catalyst as necessary. As such catalyst, mineral acids (e.g., hydrochloric acid, hydrobromic acid, sulfuric acid etc.), carboxylic acids (e.g., formic acid, acetic acid, propionic acid, trifluoroacetic acid etc.), sulfonic acids (e.g., methanesulfonic acid, p-toluenesulfonic acid etc.), Lewis acids (e.g., aluminum chloride, zinc chloride, zinc bromide, boron trifluoride, titanium chloride etc.), acetate (e.g., sodium acetate, potassium acetate etc.), molecular sieves (e.g., molecular sieves 3A, 4A, 5A etc.) can be mentioned. Sulfonic acids are preferable and p-toluenesulfonic acid is particularly preferable. The amount of the catalyst to be used is, for example, about 0.001 to about 10 mol, preferably about 0.01 to about 1 mol, per 1 mol of N-[2-oxo-2-(4-oxo-3-phenylpiperidin-1-yl)ethyl]acetamide.

While the reaction temperature varies depending on the solvent to be used, it is generally about 30° C. to about 200° C., preferably about 50° C. to about 150° C., and the reaction time is generally about 0.1 hr to about 48 hrs, preferably about 0.1 hr to about 24 hrs.

This reaction can also be promoted by azeotropic dehydration known per se.

Then, the imine is converted to an optically active compound (IX′) by hydrogenation. When (S)-1-phenylethylamine is used as optically active amine (VIII), N-[2-oxo-2-((3R,4S)-3-phenyl-4-{[(1S)-1-phenylethyl]amino}piperidin-1-yl)ethyl]acetamide can be obtained as an optically active compound (IX′).

The hydrogenation can be carried out by a method known per se, in a solvent inert to the reaction, for example, a method using a metal hydride and a method involving catalytic hydrogenation can be mentioned. Of these, catalytic hydrogenation is more preferable.

As the metal hydride, the metal hydrides exemplified in Method B, Step 1 can be mentioned.

The amount of the metal hydride to be used is, for example, about 1 to about 50 mol, preferably about 1 to about 10 mol, per 1 mol of the imine.

The solvent to be used here is not particularly limited as long as it does not adversely affect the reaction and can dissolve the starting compound and, for example, aromatic hydrocarbons such as toluene, xylene and the like; aliphatic hydrocarbons such as heptane, hexane and the like; halogenated hydrocarbons such as chloroform, dichloromethane and the like; ethers such as diethyl ether, tetrahydrofuran, dioxane and the like; alcohols such as methanol, ethanol, 2-propanol, butanol, benzyl alcohol and the like; nitriles such as acetonitrile and the like; N,N-dimethylformamide; dimethyl sulfoxide and the like can be mentioned. These solvents may be used in a mixture at an appropriate ratio. The reaction can be carried out in the state almost free of a solvent or using a solvent in an amount not more than 100-fold weight of the imine. Generally, use of a solvent in a 5- to 30-fold weight of the imine is preferable.

The reaction temperature is generally about −80° C. to about 200° C., preferably about −50° C. to about 100° C., and the reaction time is generally about 5 min. to about 72 hrs, preferably about 0.5 hr to about 12 hrs.

The catalytic hydrogenation can be carried out under a hydrogen atmosphere in the presence of a catalyst. As the catalyst to be used, the catalysts exemplified in Method B, Step 1 can be mentioned, nickel catalyst is preferable, and Raney nickel catalyst is particularly preferable. Its amount of use based on nickel is about 0.1 to about 200 mol, preferably about 1 to about 100 mol, per 1 mol of the imine.

The catalytic hydrogenation is generally carried out in a solvent inert to the reaction. As such solvent, for example, alcohols such as methanol, ethanol, propanol, butanol, benzyl alcohol and the like; aliphatic hydrocarbons such as heptane, hexane and the like; aromatic hydrocarbons such as benzene, toluene, xylene and the like; halogenated hydrocarbons such as dichloromethane, chloroform and the like; ethers such as diethyl ether, dimethoxyethane, dioxane, tetrahydrofuran and the like; esters such as ethyl acetate and the like; amides such as N,N-dimethylformamide and the like; carboxylic acids such as acetic acid and the like; water and a mixture thereof can be mentioned.

Preferable solvents are alcohols and aromatic hydrocarbons. More preferable solvent is a mixed solvent of alcohols and aromatic hydrocarbons. Particularly, preferable alcohols include ethanol and preferable aromatic hydrocarbons include toluene. Most preferred is a mixed solvent of ethanol and toluene. The reaction can be carried out in the state almost free of a solvent or using a solvent in an amount not more than 100-fold weight of the imine. Generally, use of a solvent in a 5- to 30-fold weight of the imine is preferable.

The reaction can be advantageously carried out by adding an additive as necessary. As the additive, organic amines (e.g., alkylamines such as trimethylamine, triethylamine, diisopropylethylamine, N-methylmorpholine and the like, aromatic amines such as pyridine, N,N-dimethylaniline and the like, etc.) are preferable, and of these, triethylamine and diisopropylethylamine are preferable. Its amount of use is about 0.001 to about 10 mol, preferably about 0.01 to about 5 mol, per 1 mol of the imine.

The hydrogenation can be carried out by any of a batch type reaction and a continuous reaction. The hydrogen pressure at which the reaction is carried out is generally about 0.01 to about 5 MPa, and preferably about 0.1 to about 1 MPa. The reaction temperature is generally about 0° C. to about 150° C., preferably about 20° C. to about 100° C., and the reaction time is generally about 5 min. to about 120 hrs.

While the imine obtained in Step 1 can be isolated and purified by, for example, conventional separation means such as recrystallization, distillation, chromatography and the like, it is preferable to carry out reduction without isolation. In this method, moreover, the production and reduction of the above-mentioned imine can be simultaneously carried out to directly give an optically active compound (IX′) from N-[2-oxo-2-(4-oxo-3-phenylpiperidin-1-yl)ethyl]acetamide.

(Step 2)

In this Step, compound (IX′) obtained in Step 1 is subjected to hydrogenolysis to give N-{2-[(3R,4S)-4-amino-3-phenylpiperidin-1-yl]-2-oxoethyl}acetamide or a salt thereof. The hydrogenolysis can be carried out according to a method known per se and, for example, a method by catalytic hydrogenation can be mentioned.

The catalytic hydrogenation can be carried out under a hydrogen atmosphere in the presence of a catalyst. As the catalyst to be used, for example, palladium catalysts such as palladium carbon, palladium hydroxide carbon, palladium oxide and the like; nickel catalysts such as Raney nickel catalyst and the like; platinum catalysts such as platinum oxide, platinum carbon and the like; rhodium catalysts such as rhodium carbon and the like, and the like can be mentioned. Of these catalysts, a heterogeneous catalyst supporting palladium is preferable, particularly palladium carbon, and palladium hydroxide carbon is preferable. Its amount of use is palladium about 0.0001 to about 1 mol, preferably about 0.001 to about 0.5 mol, per 1 mol of compound (IX′).

The catalytic hydrogenation is generally carried out in a solvent inert to the reaction. As such solvent, for example, alcohols such as methanol, ethanol, propanol, butanol, benzyl alcohol and the like; aliphatic hydrocarbons such as heptane, hexane and the like; aromatic hydrocarbons such as benzene, toluene, xylene and the like; halogenated hydrocarbons such as dichloromethane, chloroform and the like; ethers such as diethyl ether, dimethoxyethane, dioxane, tetrahydrofuran and the like; esters such as ethyl acetate and the like; nitrites such as acetonitrile and the like; amides such as N,N-dimethylformamide and the like; carboxylic acids such as acetic acid and the like; water and a mixture thereof can be mentioned. Preferable solvent is alcohol and, ethanol is particularly preferable. The reaction can be carried out in the state almost free of a solvent or using a solvent in an amount not more than 100-fold weight of compound (IX′). Generally, use of a solvent in a 5- to 30-fold weight of compound (IX′) is preferable.

The hydrogenation can be carried out by any of a batch type reaction and a continuous reaction. The hydrogen pressure at which the reaction is carried out is, for example, generally about 0.1 to about 5 MPa, and preferably about 0.1 to about 1 MPa. The reaction temperature is generally about 0° C. to about 200° C., preferably about 20° C. to about 60° C., and the reaction time is generally about 5 min. to about 120 hrs.

In this method, without isolating compound (IX′), which is as an intermediate, the next hydrogenolysis can be also carried out to directly give N-{2-[(3R,4S)-4-amino-3-phenylpiperidin-1-yl]-2-oxoethyl}acetamide or a salt thereof from N-[2-oxo-2-(4-oxo-3-phenylpiperidin-1-yl)ethyl]acetamide.

N-{2-[(3R,4S)-4-amino-3-phenylpiperidin-1-yl]-2-oxoethyl}acetamide can be isolated and purified by, for example, conventional separation means such as recrystallization, distillation, chromatography and the like.

In the above-mentioned method, when N-{2-[(3R,4S)-4-amino-3-phenylpiperidin-1-yl]-2-oxoethyl}acetamide is obtained as a free compound, for example, a salt with an inorganic acid (e.g., hydrochloric acid, sulfuric acid, hydrobromic acid etc.) or an organic acid (e.g., methanesulfonic acid, benzenesulfonic acid, toluenesulfonic acid, oxalic acid, fumaric acid, maleic acid, tartaric acid etc.) can be produced according to a conventional method. When N-{2-[(3R,4S)-4-amino-3-phenylpiperidin-1-yl]-2-oxoethyl}acetamide is obtained in the form of a salt, it can be converted to a free compound or other salt according to a conventional method. N-{2-[(3R,4S)-4-Amino-3-phenylpiperidin-1-yl]-2-oxoethyl}acetamide is preferably obtained in the form of a salt with an organic acid, most preferably as a methanesulfonate. The amount of the acid to be use in the formation of the methanesulfonate is, for example, about 0.9 to about 5 mol, preferably about 0.9 to about 2 mol, per 1 mol of N-{2-[(3R,4S)-4-amino-3-phenylpiperidin-1-yl]-2-oxoethyl}acetamide.

N-{2-[(3R,4S)-4-Amino-3-phenylpiperidin-1-yl]-2-oxoethyl}acetamide methanesulfonate obtained by this method has an extremely high chemical purity (not less than 99%), enantiomer excess (not less than 99.5% ee) and diastereomer excess (not less than 99.5% de), and the compound has high quality.

N-[2-Oxo-2-(4-oxo-3-phenylpiperidin-1-yl)ethyl]acetamide to be used as a starting compound in Method D, Step 1 can be produced by, for example, reacting 3-phenylpiperidin-4-one or a salt thereof with N-acetylglycine or a salt thereof or reactive derivative thereof which is an acylating agent.

As the reactive derivative of N-acetylglycine or a salt thereof, for example, a compound represented by the formula (IIa′)
CH₃CONHCH₂—(C═O)-L′ (IIa′)
wherein L′ is a leaving group, or a salt thereof (hereinafter to be referred to as reactive derivative (IIa′)) can be used.

As the leaving group for L′, those exemplified as the leaving group for L in reactive derivative (IIa) used in Method A can be mentioned.

When N-acetylglycine or a salt thereof is used as an acylating agent, for example, it can be produced by the use of a condensing agent. As the “condensing agent”, for example, N,N′-dicyclohexylcarbodiimide, 1-ethyl-3-(3-dimethylaminopropyl)carbodiimide hydrochloride, carbonyldiimidazole, di-(N-succinimidyl)carbonate, N-ethyl-5-phenylisoxazolium-3′-sulfonate, 1H-benzotriazol-1-yloxytris(dimethylamino)phosphonium hexafluorophosphate, O-(benzotriazol-1-yl)-N,N,N′,N′-tetramethyluronium hexafluorophosphate, an organophosphorus compound and the like can be mentioned.

The “organophosphorus compound” is reacted, for example, in the presence of a base according to a method described in JP-A-58-43979. As the “organophosphorus compound”, for example alkyl o-phenylenephosphate such as methyl phenylenephosphate, ethyl o-phenylenephosphate (EPPA) and the like, aryl o-phenylenephosphate such as phenyl o-phenylenephosphate, p-chlorophenyl o-phenylenephosphate and the like, diphenylphosphoryl azide and the like can be mentioned.

The amount of the “condensing agent” to be used is generally about 1 to about 10 mol, preferably about 1 to about 3 mol, per 1 mol of 3-phenylpiperidin-4-one or a salt thereof.

This reaction is generally carried out in a solvent, and a convenient base may be added to promote the reaction. As the solvent, for example, hydrocarbons such as benzene, toluene and the like; ethers such as ethyl ether, dioxane, tetrahydrofuran and the like; esters such as ethyl acetate and the like; halogenated hydrocarbons such as chloroform, dichloromethane and the like; esters such as ethyl acetate and the like; amides such as N,N-dimethylformamide and the like; aromatic amines such as pyridine and the like; nitrites such as acetonitrile and the like; water and the like can be mentioned. These solvents may be used in a mixture at an appropriate ratio.

As the base, for example, alkali metal hydroxides such as sodium hydroxide, potassium hydroxide and the like; hydrogencarbonates such as sodium hydrogencarbonate, potassium hydrogencarbonate and the like; carbonates such as sodium carbonate, potassium carbonate and the like; acetates such as sodium acetate and the like; tertiary amines such as trimethylamine, triethylamine, N-methylmorpholine and the like; aromatic amines such as pyridine, picoline, N,N-dimethylaniline and the like, and the like can be mentioned. The amount of the base to be used is, for example, about 0.5 to about 100 mol, preferably about 0.5 to about 10 mol, per 1 mol of 3-phenylpiperidin-4-one or a salt thereof.

The amount of the acylating agent to be used is generally about 1 to about 10 mol, preferably about 1 to about 3 mol, per 1 mol of 3-phenylpiperidin-4-one or a salt thereof. The reaction temperature is generally about −10° C. to about 150° C., preferably about 0° C. to about 100° C., and the reaction time is generally about 15 min. to about 24 hrs, preferably about 30 min. to about 16 hrs.

As the acylating agent, N-acetylglycine is most preferable. In this case, a method using a condensing agent is preferable, and addition of a base here is more preferable. Especially, a method using 1-ethyl-3-(3-dimethylaminopropyl)carbodiimide hydrochloride as a condensing agent and adding triethylamine as a base is most preferable. In this case, acetonitrile is preferable as the solvent.

When compound (I) is obtained in a free compound in the above-mentioned method, a salt with for example, inorganic acids (e.g., hydrochloric acid, sulfuric acid, hydrobromic acid etc.), organic acids (e.g., methanesulfonic acid, benzenesulfonic acid, toluenesulfonic acid, oxalic acid, fumaric acid, maleic acid, tartaric acid etc.), inorganic bases (e.g., alkali metals such as sodium, potassium etc., alkaline earth metals such as calcium, magnesium etc., aluminum, ammonium, and the like), or organic bases (e.g., trimethylamine, triethylamine, pyridine, picoline, ethanolamine, diethanolamine, triethanolamine, dicyclohexylamine, N,N′-dibenzylethylenediamine etc.) and the like can be prepared in a routine manner. When compound (I) is obtained in the form of a salt, the compound can be converted to a free compound or another salt in a routine manner.

In addition, when the starting compound may form a salt in each of the above-mentioned reactions, the compound may be used as a salt. Such salt includes, for example, those exemplified as a salt of compound (I).

Compound (I) prepared by such methods can be isolated and purified by a typical separation means such as recrystallization, distillation, chromatography etc.

When compound (I) contains an optical isomer, a stereoisomer, a regioisomer or a rotamer, these are also included in compound (I), and can be obtained as a single product according to synthesis and separation methods known per se (for example, concentration, solvent extraction, column chromatography, recrystallization etc.). For example, when compound (I) has an optical isomer, the optical isomer resolved from this compound is also included in compound (I).

The optical isomer can be prepared by a method known per se. To be specific, an optically active synthetic intermediate is used, or the final racemate product is subjected to optical resolution according to a conventional method to give an optical isomer.

The method of optical resolution may be a method known per se, such as a fractional recrystallization method, a chiral column method, a diastereomer method etc.

1) Fractional Recrystallization Method

A method wherein a salt of a racemate with an optically active compound (e.g., (+)-mandelic acid, (−)-mandelic acid, (+)-tartaric acid, (−)-tartaric acid, (+)-1-phenethylamine, (−)-1-phenethylamine, cinchonine, (−)-cinchonidine, brucine etc.) is formed, which is separated by a fractional recrystallization method, and if desired, a free optical isomer is obtained by a neutralization step.

2) Chiral Column Method

A method wherein a racemate or a salt thereof is applied to a column for separation of an optical isomer (a chiral column) to allow separation. In the case of a liquid chromatography, for example, a mixture of the optical isomers is applied to a chiral column such as ENANTIO-OVM (manufactured by Tosoh Corporation), CHIRAL series (manufactured by Daicel Chemical Industries, Ltd.) and the like, and developed with water, various buffers (e.g., phosphate buffer) and organic solvents (e.g., ethanol, methanol, 2-propanol, acetonitrile, trifluoroacetic acid, diethylamine etc.) solely or in admixture to separate the optical isomer. In the case of a gas chromatography, for example, a chiral column such as CP-Chirasil-DeX CB (manufactured by GL Sciences Inc.) and the like is used to allow separation.

3) Diastereomer Method

A method wherein a racemic mixture is prepared into a diastereomeric mixture by chemical reaction with an optically active reagent, which is made into a single substance by a typical separation means (e.g., a fractional recrystallization method, a chromatography method etc.) and the like, and is subjected to a chemical treatment such as hydrolysis and the like to separate an optically active reagent moiety, whereby an optical isomer is obtained. For example, when compound (I) contains hydroxy, or primary or secondary amino group within a molecule, the compound and an optically active organic acid (e.g., MTPA [α-methoxy-α-(trifluoromethyl)phenylacetic acid], (−)-menthoxyacetic acid etc.) and the like are subjected to condensation reaction to give diastereomers of the ester compound or the amide compound, respectively. When compound (I) has a carboxyl group, this compound and an optically active amine or an optically active alcohol reagent are subjected to condensation reaction to give diastereomers of the ester compound or the amide compound, respectively. The separated diastereomer is converted to an optical isomer of the original compound by acid hydrolysis or base hydrolysis.

Compound (I) may be in the form of a crystal.

The crystal of compound (I) can be prepared by crystallization of compound (I) by a method of crystallization known per se.

Examples of the method of crystallization include a method of crystallization from a solution, a method of crystallization from vapor, a method of crystallization from the melts, and the like.

The “method of crystallization from a solution” is typically a method of shifting a non-saturated state to supersaturated state by varying factors involved in solubility of compounds (solvent composition, pH, temperature, ionic strength, redox state etc.) or the amount of solvent. To be specific, for example, a concentration method, a slow cooling method, a reaction method (a diffusion method, an electrolysis method), a hydrothermal growth method, a flux method and the like can be mentioned. Examples of the solvent to be used include aromatic hydrocarbons (e.g., benzene, toluene, xylene etc.), halogenated hydrocarbons (e.g., dichloromethane, chloroform etc.), saturated hydrocarbons (e.g., hexane, heptane, cyclohexane etc.), ethers (e.g., diethyl ether, diisopropyl ether, tetrahydrofuran, dioxane etc.), nitriles (e.g., acetonitrile etc.), ketones (e.g., acetone etc.), sulfoxides (e.g., dimethyl sulfoxide etc.), acid amides (e.g., N,N-dimethylformamide etc.), esters (e.g., ethyl acetate etc.), alcohols (e.g., methanol, ethanol, isopropyl alcohol etc.), water and the like. These solvents are used alone or in a combination of two or more at a suitable ratio (e.g., 1:1 to 1:100 (a volume ratio)). Where necessary, a seed crystal can also be used.

The “method of crystallization from vapor” is, for example, a vaporization method (a sealed tube method, and a gas stream method), a gas phase reaction method, a chemical transportation method and the like.

The “method of crystallization from the melts” is, for example, a normal freezing method (a pulling method, a temperature gradient method and a Bridgman method), a zone melting method (a zone leveling method and a floating zone method), a special growth method (a VLS method and a liquid phase epitaxy method) and the like.

Preferable examples of the method of crystallization include a method of dissolving compound (I) in a suitable solvent (e.g., alcohols such as methanol, ethanol etc. and the like) at a temperature of 20° C. to 120° C., and cooling the resulting solution to a temperature not higher than the temperature of dissolution (e.g., 0° C. to 50° C., preferably 0° C. to 20° C.) and the like.

The thus-obtained crystal of the present invention can be isolated, for example, by filtration and the like.

As an analysis method of the obtained crystal, crystal analysis by powder X-ray diffraction is generally employed. Moreover, as a method for determining the crystal orientation, a mechanical method, an optical method and the like can also be mentioned.

The crystal of compound (I) obtained in the above-mentioned production method (hereinafter to be abbreviated as “crystal of the present invention”) has high purity, high quality and low hygroscopicity, is free of denaturation even after a long-term preservation under normal conditions, and is extremely superior in stability. The crystal is also superior in biological properties (e.g., in vivo kinetics (absorbability, distribution, metabolism, excretion), efficacy expression etc.), and is extremely useful as a pharmaceutical agent.

As the crystal of the present invention, the crystal of compound A (preferably free form) is preferably used.

As the crystal of compound A, for example, a crystal (crystal Form A) having a melting point of about 107° C. to about 119° C., and a diffraction pattern having characteristic peaks at lattice spacing (d value) of about 5.83, about 5.17, about 4.61, about 4.00 and about 3.40 angstroms by powder X-ray diffraction can be mentioned.

In addition, a crystal (crystal Form B) having a melting point of about 124° C. to about 134° C., and a diffraction pattern having characteristic peaks at lattice spacing (d value) of about 7.26, about 4.61, about 4.54, about 4.38 and about 3.63 angstroms by powder X-ray diffraction can be mentioned.

While the crystal of compound A of the present invention can be obtained by applying the “method of crystallization” exemplified for compound (I), application of the “method of crystallization from solution” is more preferable.

The above-mentioned “crystal Form A” is desirably precipitated from a supersaturation state at a low temperature. The temperature of the supersaturation state is preferably less than 46° C., more preferably not more than 30° C., and most preferably not more than 20° C. In the supersaturation state, a crystal having a melting point of about 107° C. to 119° C. may be added as a seed crystal where necessary. For crystal precipitation, the “method of crystallization” exemplified for compound (I) can be applied, application of the “method of crystallization from solution”, is more preferable. As the solvent to be used, aromatic hydrocarbons (e.g., benzene, toluene, xylene etc.), halogenated hydrocarbons (e.g., dichloromethane, chloroform etc.), saturated hydrocarbons (e.g., hexane, heptane, cyclohexane etc.), ethers (e.g., diethyl ether, diisopropyl ether, tetrahydrofuran, dioxane etc.), nitrites (e.g., acetonitrile etc.), ketones (e.g., acetone etc.), sulfoxides (e.g., dimethyl sulfoxide etc.), acid amides (e.g., N,N-dimethylformamide etc.), esters (e.g., ethyl acetate etc.), alcohols (e.g., methanol, ethanol, isopropyl alcohol etc.), water and the like can be mentioned.

As a method for achieving a supersaturation state by the “method of crystallization from solution”, a method comprising dissolving compound A of the present invention in a solvent having a high compound A solubility and then adding a solvent having a low compound A solubility is more preferable. A method comprising dissolving compound A in ethanol as a solvent having a high compound A solubility and adding water and a method comprising dissolving compound A in ethyl acetate as a solvent having a high compound A solubility and adding diisopropyl ether or heptane are more preferable. Of these, a method comprising dissolving compound A in ethyl acetate as a solvent having a high compound A solubility and adding heptane is most preferable. The crystal thus obtained can be isolated, for example, by filtration and the like.

The above-mentioned “crystal Form B” is desirably precipitated from a supersaturation state at a high temperature. The temperature of the supersaturation state is preferably not less than 46° C., more preferably not less than 50° C., and most preferably not less than 55° C. In the supersaturation state, a crystal having a melting point of about 124° C. to 134° C. may be added as a seed crystal where necessary. For crystal precipitation, the “method of crystallization” exemplified for compound (I) can be applied, application of the “method of crystallization from solution” is more preferable. As the solvent to be used, aromatic hydrocarbons (e.g., benzene, toluene, xylene etc.), halogenated hydrocarbons (e.g., dichloromethane, chloroform etc.), saturated hydrocarbons (e.g., hexane, heptane, cyclohexane etc.), ethers (e.g., diethyl ether, diisopropyl ether, tetrahydrofuran, dioxane etc.), nitrites (e.g., acetonitrile etc.), ketones (e.g., acetone etc.), sulfoxides (e.g., dimethyl sulfoxide etc.), acid amides (e.g., N,N-dimethylformamide etc.), esters (e.g., ethyl acetate etc.), alcohols (e.g., methanol, ethanol, isopropyl alcohol etc.), water and the like can be mentioned.

As a method for achieving a supersaturation state by the “method of crystallization from solution”, a method comprising dissolving compound A of the present invention in a solvent having a high compound A solubility and then adding a solvent having a low compound A solubility is more preferable. A method comprising dissolving compound A in tetrahydrofuran as a solvent having a high compound A solubility and adding diisopropyl ether or heptane is more preferable. The crystal thus obtained can be isolated, for example, by filtration and the like.

The crystal of compound A has high purity (purity not less than 99%), high quality and low hygroscopicity, is free of denaturation even after a long-term preservation under normal conditions, and is extremely superior in stability. The crystal is also superior in biological properties (e.g., in vivo kinetics (absorbability, distribution, metabolism, excretion), efficacy expression etc.), and is extremely useful as a pharmaceutical agent.

In the present specification, the optical rotation ([α]_{D}) means that measured using, for example, polarimeter (JASCO Corporation (JASCO), P-1030 polarimeter (No. AP-2)) and the like.

In the present specification, the melting point means that measured using, for example, a micromelting point apparatus (Yanako, MP-500D), a DSC (differential scanning calorimetry) device (SEIKO, EXSTAR 6000) and the like.

In the present specification, the peak by powder X-ray diffraction means that measured using, for example, RINT Ultima⁺ 2100 (Rigaku Corporation) etc. with Cu-K_{α} ray and the like as a radiation source.

In general, the melting point and the peak by powder X-ray diffraction may vary depending on the measurement apparatuses, the measurement conditions and the like. The crystal in the present specification may show different values from the melting point or the peak by powder X-ray diffraction described in the present specification, as long as it is within each of a general error range.

Compound (I) of the present invention has an excellent antagonistic action for tachykinin receptors, particularly Substance P receptor antagonistic action, neurokinin A receptor antagonistic action, in addition to inhibitory action for the increased extravasation in trachea induced by capsaicin. The compound of the present invention has low toxicity and thus it is safe.

Accordingly, the compounds of the present invention having excellent antagonistic actions for Substance P receptors and neurokinin A receptors etc. can be used as a safe pharmaceutical composition for preventing and treating the following diseases related to Substance P in mammals (e.g., mice, rats, hamsters, rabbits, cats, dogs, bovines, sheep, monkeys, humans etc.).

(1) Lower urinary tract diseases [for example, lower urinary tract disease associated with overactive bladder and benign prostatic hyperplasia, pelvic visceral pain, lower urinary tract disease associated with chronic prostatitis, lower urinary tract disease associated with interstitial cystitis and the like]

(2) Gastrointestinal diseases [for example, irritable bowel syndrome, inflammatory bowel disease, ulcerative colitis syndrome, Crohn's disease, diseases caused by a spiral urease-positive gram-negative bacterium (e.g., Helicobacter pylori etc.) (e.g., gastritis, gastric ulcer etc.), gastric cancer, postgastrostomy disorder, dyspepsia, esophageal ulcer, pancreatitis, polyp of the colon, cholelithiasis, hemorrhoids, peptic ulcer, situational ileitis, vomiting, nausea etc.]

(3) Inflammatory or allergic diseases [for example, allergic rhinitis, conjunctivitis, gastrointestinal allergy, pollinosis, anaphylaxis, dermatitis, herpes, psoriasis, bronchitis, expectoration, retinopathy, postoperative and posttraumatic inflammation, regression of puffiness, pharyngitis, cystitis, meningitidis, inflammatory ophthalmic diseases etc.]

(4) Osteoarthropathy diseases [for example, rheumatoid arthritis (chronic rheumatoid arthritis), arthritis deformans, rheumatoid myelitis, osteoporosis, abnormal growth of cells, bone fracture, bone refracture, osteomalacia, osteopenia, osseous Behcet's disease, rigid myelitis, articular tissue destruction by gonarthrosis deformans and similar diseases thereto etc.]

(5) Respiratory diseases [for example, cold syndrome, pneumonia, asthma, pulmonary hypertension, pulmonary thrombi/pulmonary obliteration, pulmonary sarcoidosis, pulmonary tuberculosis, interstitial pneumonia, silicosis, adult tachypnea syndrome, chronic obliterative pulmonary diseases, cough etc.]

(6) Infectious diseases [HIV infectious diseases, virus infectious diseases due to cytomegalo virus, influenza virus, herpes virus and the like, rickettsia infectious diseases, bacterial infectious diseases, sexually-transmitted diseases, carinii pneumonia, helicobacter pylori infectious disease, systemic fungal infectious diseases, tuberculosis, invasive staphylococcal infectious diseases, acute viral encephalitis, acute bacterial meningitidis, AIDS encephalitis, septicemia, sepsis, sepsis gravis, septic shock, endotoxin shock, toxic shock syndromes etc.]

(7) Cancers [for example, primary, metastatic or recurrent breast cancer, prostatic cancer, pancreatic cancer, gastric cancer, lung cancer, colorectal cancer (colon cancer, rectal cancer, anal cancer), esophagus cancer, duodenal cancer, head and neck cancer (tongue cancer, pharynx cancer, larynx cancer), brain tumor, neurinoma, non-small cell lung cancer, small cell lung cancer, hepatic cancer, renal cancer, colic cancer, uterine cancer (cancer of the uterine body, uterine cervical cancer), ovarian cancer, bladder cancer, skin cancer, hemangioma, malignant lymphoma, malignant melanoma, thyroid cancer, bone tumor, angiofibroma, retinosarcoma, penis cancer, pediatric solid cancer, Kaposi's sarcoma, Kaposi's sarcoma caused by AIDS, tumor of the maxillary sinus, fibrous histiocytoma, smooth muscle sarcoma, rhabdomyosarcoma, liposarcoma, fibroid tumors of the uterus, osteoblastoma, osteosarcoma, chondrosarcoma, carcinomatous mesothelial tumor, tumors such as leukemia, Hodgkin's disease etc.]

(8) Central nervous system diseases [for example, neurodegenerative diseases (e.g., Alzheimer's disease, Down's disease, Parkinson's disease, Creutzfeldt-Jakob's disease, amyotrophic lateral sclerosis (ALS), Huntington chorea, diabetic neuropathy, multiple sclerosis etc.), mental diseases (e.g., schizophrenia, depression, mania, anxiety neurosis, obsessive-compulsive neurosis, panic disorder, epilepsy, alcohol dependence, anxiety, anxious mental state etc.), central and peripheral nerve disorders (e.g., head trauma, spinal cord injury, brain edema, disorders of sensory function, abnormality of sensory function, disorders of autonomic nervous function and abnormality of autonomic nervous function, whiplash injury etc.), memory disorders (e.g., senile dementia, amnesia, cerebrovascular dementia etc.), cerebrovascular disorders (e.g., disorders and aftereffect and/or complication from intracerebral hemorrhage, brain infarction etc., asymptomatic cerebro-vascular accident, transient cerebral ischemic attack, hypertensive encephalopathia, blood-brain barrier disorder etc.), recurrence and aftereffect of cerebro-vascular accident (e.g., neural symptoms, mental symptoms, subjective symptoms, disorders of daily living activities etc.), post-cerebrovascular occlusion central hypofunction, disorder or abnormality of cerebral circulation and/or autoregulation of renal circulation, sleep disorder (insomnia) etc.]

(9) Circulatory diseases [for example, acute coronary artery syndromes (e.g., acute cardiac infarction, unstable angina etc.), peripheral arterial obstruction, Raynaud's disease, Buerger disease, restenosis after coronary-artery intervention (percutaneous transluminal coronary angioplasty (PTCA), directional coronary atherectomy (DCA), stenting etc.), restenosis after coronary-artery bypass operation, restenosis after intervention (angioplasty, atherectomy, stenting etc.) or bypass operation in other peripheral artery, ischemic cardiac diseases (e.g., cardiac infarction, angina etc.), myocarditis, intermittent claudication, lacunar infarction, arteriosclerosis (e.g., atherosclerosis etc.), cardiac failure (acute cardiac failure, chronic cardiac failure accompanied by congestion), arrhythmia, progress of atherosclerotic plaque, thrombosis, hypertension, hypertensive tinnitus, hypotension etc.]

(10) Pains [e.g., migraine, neuralgia, pelvic visceral pain (including cystalgia) etc.]

(11) Autoimmune diseases [for example, collagen disease, systemic lupus erythematosus, scleroderma, polyarteritis, myasthenia gravis, multiple sclerosis, Sjogren's syndrome, Behcet's disease etc.]

(12) Hepatic diseases [e.g., hepatitis (including chronic hepatitis), cirrhosis, interstitial hepatic diseases etc.]

(13) Pancreatic diseases [e.g., pancreatitis (including chronic pancreatitis) etc.]

(14) Renal diseases [e.g., nephritis, glomerulonephritis, glomerulosclerosis, renal failure, thrombotic microangiopathy, dialysis complications, organ disorders including nephropathia by radiation, diabetic nephropathia etc.]

(15) Metabolic diseases [e.g., diabetic diseases (insulin-dependent diabetes, diabetic complications, diabetic retinopathy, diabetic microangiopathy, diabetic neuropathy etc.), glucose tolerance abnormality, obesity, benign prostatic hyperplasia, sexual dysfunction etc.]

(16) Endocrine diseases [e.g., Addison's disease, Cushing's syndrome, melanocytoma, primary aldosteronism etc.]

(17) Other Diseases

(a) Transplant rejection [e.g., posttransplantational rejection, posttransplantational polycythemia, hypertension, organ disorder and/or vascular hypertrophy, graft-versus-host disease etc.]

(b) Abnormality in characteristic of blood and/or blood components [e.g., enhancement in platelet aggregation, abnormality of erythrocyte deformability, enhancement in leukocyte adhesiveness, increase in blood viscosity, polycythemia, vascular peliosis, autoimmune hemolytic anemia, disseminated intravascular coagulation syndrome (DIC), multiple myelopathy etc.]

(c) Gynecologic diseases [e.g., climacteric disorder, gestational toxicosis, endometriosis, hysteromyoma, ovarian disease, mammary disease etc.]

(d) Dermatic diseases [e.g., keloid, angioma, psoriasis, pruritus etc.]

(e) Ophthalmic diseases [e.g., glaucoma, ocular hypertension disease etc.]

(f) Otolaryngological diseases [e.g., Menuel syndrome, tinnitus, gustation disorder, dizziness, disequilibrium, dysphagia etc.]

(g) Diseases due to environmental and/or occupational factors (e.g., radiation disorder, disorders by ultraviolet ray-infrared ray-laser ray, altitude sickness etc.)

(h) Ataxia

(i) Chronic Fatigue Syndrome

Of these diseases, the compound of the present invention is particularly useful as a tachykinin receptor antagonist, an agent for improving lower urinary tract diseases such as urinary frequency, urinary incontinence and the like or a therapeutic drug for these lower urinary tract diseases.

Pharmaceutical preparations comprising the compound of the present invention may be in any solid forms of powders, granules, tablets, capsules, suppositories etc., and in any liquid forms of syrups, emulsions, injections, suspensions etc.

The pharmaceutical preparations comprising the compound of the present invention can be produced by any conventional methods, for example, blending, kneading, granulation, tabletting, coating, sterilization, emulsification etc., in accordance with the forms of the preparations to be produced. For the production of such pharmaceutical preparations, for example, each of the items in General Principles for pharmaceutical preparations in the Japanese Pharmacopeia, can be made reference to. In addition, the pharmaceutical preparations of the present invention may be formulated into a sustained release preparation containing active ingredients and biodegradable polymer compounds. The sustained release preparation can be produced according to the method described in JP-A-9-263545.

In the pharmaceutical preparations of the present invention, the content of the compound or a salt thereof in the present invention varies depending on the forms of the preparations, but is generally about 0.01 to 100% by weight, preferably about 0.1 to 50% by weight, more preferably 0.5 to 20% by weight, relative to the total weight of each preparation.

When the compound of the present invention is used in the above-mentioned pharmaceutical preparations, it may be used alone, or in admixture with a suitable, pharmaceutically acceptable carrier, for example, excipients (e.g., starch, lactose, sucrose, calcium carbonate, calcium phosphate etc.), binders (e.g., starch, arabic gum, carboxymethyl cellulose, hydroxypropyl cellulose, crystalline cellulose, alginic acid, gelatin, polyvinyl pyrrolidone etc.), lubricants (e.g., stearic acid, magnesium stearate, calcium stearate, talc etc.), disintegrants (e.g., calcium carboxymethylcellulose, talc etc.), diluents (e.g., water for injection, physiological saline etc.) and if desired, with the additives (e.g., a stabilizer, a preservative, a colorant, a fragrance, a dissolution aid, an emulsifier, a buffer, an isotonic agent etc.) and the like, by ordinary methods. It can be formulated into the solid preparations such as powders, fine granules, granules, tablets, capsules etc., or into the liquid preparations such as injections etc., and can be administered orally or parenterally.

The dose of the pharmaceutical preparation of the present invention varies depending on the kinds of the compound of the present invention or a pharmaceutically acceptable salt thereof, the administration route, the condition and the age of patients etc. For example, the dose for oral administration of the pharmaceutical preparation to an adult patient suffering from lower urinary tract symptoms is generally from about 0.005 to 50 mg/kg body/day, preferably from about 0.05 to 10 mg/kg body/day, more preferably from about 0.2 to 4 mg/kg body/day, based on the compound of the present invention, which may be administered once a day or in two or three divided portions a day.

The dose when the pharmaceutical composition of the present invention is a sustained release preparation varies depending on the kinds and the content of compound (I), the formulation, the duration time of drug release, the animals to be administered (e.g., mammals such as humans, rats, mice, cats, dogs, rabbits, bovines, swines etc.), and the object of administration. For example, when it is parenterally administered, preferably about 0.1 to about 100 mg of compound (I) is released from the preparation for 1 week.

The compound of the present invention can be used in a mixture or combination with other pharmaceutically active ingredients at a suitable ratio.

Combination of the compound of the present invention with other pharmaceutically active ingredients can give the following excellent effects:

(1) a dose can be reduced as compared with separate administration of the compound of the present invention or other pharmaceutically active ingredients. More specifically, when the compound of the present invention is combined with anticholinergic agents or NK-2 receptor antagonists, the dose can be reduced as compared with separate administration of anticholinergic agents or NK-2 receptor antagonists, and therefore, side effects such as dry mouth can be reduced;

(2) according to symptoms of patient (mild symptoms, severe symptoms etc.), a drug to be combined with the compound of the present invention can be selected;

(3) by choosing other pharmaceutically active ingredients which have different mechanism of action from that of the compound of the present invention, the therapeutic period can be designed longer;

(4) by choosing other pharmaceutically active ingredients which have different mechanism of action from that of the compound of the present invention, continuation of therapeutic effects can be obtained; and

(5) by combining the compound of the present invention and other pharmaceutically active ingredients, excellent effects such as synergic effects can be obtained.

A drug which is mixed or combined with the compound of the present invention (hereinafter, briefly referred to as concomitant drugs) includes the following.

(1) Agent for Treating Diabetes

Insulin preparations (e.g., animal insulin preparations extracted from the bovine or swine pancreas; human insulin preparations synthesized by a genetic engineering technique using Escherichia coli or a yeast; insulin zinc; protamine zinc insulin; a fragment or a derivative of insulin (e.g., INS-1 etc.) etc.), agents for potentiating insulin sensitivity (e.g., pioglitazone hydrochloride, troglitazone, rosiglitazone or its maleate, JTT-501, MCC-555, YM-440, GI-262570, KRP-297, FK-614, CS-011 etc.), α-glucosidase inhibitors (e.g., voglibose, acarbose, miglitol, emiglitate etc.), biguanides (e.g., phenformin, metformin, buformin etc.), sulfonylureas (e.g., tolbutamide, glibenclamide, gliclazide, chlorpropamide, tolazamide, acetohexamide, glyclopyramide, glimepiride etc.) and other insulin secretagogues (e.g., repaglinide, senaglinide, mitiglinide or its calcium salt hydrate, GLP-1, nateglinide etc.), dipeptidylpeptidase. IV inhibitors (e.g., NVP-DPP-278, PT-100, P32/98 etc.), β₃ agonists (e.g., CL-316243, SR-58611-A, UL-TG-307, AJ-9677, AZ40140 etc.), amylin agonists (e.g., pramlintide etc.), phosphotyrosine phosphatase inhibitors (e.g., vanadic acid etc.), gluconeogenesis inhibitors (e.g., glycogen phosphorylase inhibitors, glucose-6-phosphatase inhibitors, glucagon antagonists etc.), SGLT (sodium-glucose cotransporter) inhibitors (e.g., T-1095 etc.) and the like.

(2) Agent for Treating Diabetic Complications

Aldose reductase inhibitors (e.g., tolrestat, epalrestat, zenarestat, zopolrestat, fidarestat (SNK-860), minalrestat (ARI-509), CT-112 etc.), neurotrophic factors (e.g., NGF, NT-3 etc.), AGE inhibitors (e.g., ALT-945, pimagedine, pyratoxathine, N-phenacylthiazolium bromide (ALT-766), EXO-226 etc.), active oxygen scavengers (e.g., thioctic acid etc.), cerebral vasodilators (e.g., tiapuride etc.) and the like.

(3) Antihyperlipidemic Agent

Statin compounds inhibiting cholesterol synthesis (e.g., pravastatin, simvastatin, lovastatin, atorvastatin, fluvastatin, cerivastatin or their salt (e.g., sodium salt etc.) and the like), squalene synthase inhibitors, fibrate compounds having triglyceride lowering action (e.g., bezafibrate, clofibrate, simfibrate, clinofibrate etc.) and the like.

(4) Hypotensive Agent

Angiotensin converting enzyme inhibitors (e.g., captopril, enalapril, delapril etc.), angiotensin II antagonists (e.g., losartan, candesartan cilexetil etc.), calcium antagonists (e.g., manidipine, nifedipine, amlodipine, efonidipine, nicardipine etc.), clonidine and the like.

(5) Antiobesity Agent

Antiobesity drugs acting on the central nervous system (e.g. dexfenfluramine, fenfluramine, phentermine, sibutramine, anfepramone, dexamphetamine, mazindol, phenylpropanolamine, clobenzorex etc.), pancreatic lipase inhibitors (e.g. orlistat etc.), β₃ agonists (e.g. CL-316243, SR-58611-A, UL-TG-307, AJ-9677, AZ40140 etc.), anorectic peptides (e.g. leptin, CNTF (Ciliary Neurotrophic Factor) etc.), cholecystokinin agonists (e.g. lintitript, FPL-15849 etc.), cannabinoid CB1 receptor antagonists (e.g., rimonabant) and the like.

(6) Diuretic Agent

Xanthine derivatives (e.g., theobromine sodium salicylate, theobromine calcium salicylate etc.), thiazide preparations (e.g., ethiazide, cyclopenthiazide, trichlormethiazide, hydrochlorothiazide, hydroflumethiazide, benzylhydrochlorothiazide, penflutizide, polythiazide, methyclothiazide etc.), antialdosterone preparations (e.g., spironolactone, triamterene etc.), carbonic anhydrase inhibitors (e.g., acetazolamide etc.), chlorobenzenesulfonamide preparations (e.g., chlotthalidone, mefruside, indapamide etc.), azosemide, isosorbide, ethacrynic-acid, piretanide, bumetanide, furosemide and the like.

(7) Chemotherapeutic Agent

Alkylating agents (e.g., cyclophosphamide, ifosfamide etc.), metabolic antagonists (e.g., methotrexate, 5-fluorouracil etc.), antitumor antibiotics (e.g., mitomycin, adriamycin etc.), plant-derived antitumor agents (e.g., vincristine, vindesine, taxol etc.), cisplatin, carboplatin, etoposide and the like. Among these, 5-fluorouracil derivatives such as Furtulon and Neo-Furtulon are preferred.

(8) Immunotherapeutic Agent

Microorganism- or bacterium-derived components (e.g., muramyl dipeptide derivatives, Picibanil etc.), immunopotentiator polysaccharides (e.g., lentinan, schizophyllan, krestin etc.), genetically engineered cytokines (e.g., interferons, interleukins (IL) etc.), colony stimulating factors (e.g., granulocyte colony stimulating factor, erythropoietin etc.) and the like. Among these, interleukins such as IL-1, IL-2, IL-12 etc. are preferred.

(9) Therapeutic Agent Recognized to Ameliorate Cachexia in Animal Models or Clinical Practice

Progesterone derivatives (e.g., Megestrol acetate) [Journal of Clinical Oncology, vol. 12, pp. 213-225, 1994], metoclopramide pharmaceuticals, tetrahydrocannabinol pharmaceuticals (the above reference is applied to both), fat metabolism ameliorating agents (e.g., eicosapentanoic acid etc.) [British Journal of Cancer, vol. 68, pp. 314-318, 1993], growth hormones, IGF-1, antibodies to the cachexia-inducing factors such as TNF-α, LIF, IL-6 and oncostatin M, and the like.

(10) Antiinflammatory Agent

Steroids (e.g., dexamethasone etc.), sodium hyaluronate, cyclooxygenase inhibitors (e.g., indomethacin, ketoprofen, loxoprofen, meloxicam, ampiroxicam, celecoxib, rofecoxib etc.) and the like.

(11) Miscellaneous

Glycosylation inhibitors (e.g., ALT-711 etc.), nerve regeneration promoting drugs (e.g., Y-128, VX853, prosaptide etc.), drugs acting on the central nervous system (e.g., antidepressants such as desipramine, amitriptyline, imipramine, fluoxetine, paroxetine, doxepin, duloxetine, venlafaxine etc.), anticonvulsants (e.g., lamotrigine, carbamazepine, gabapentin), antiarrhythmic drugs (e.g., mexiletine), acetylcholine receptor ligands (e.g., ABT-594), endothelin receptor antagonists (e.g., ABT-627), monoamine uptake inhibitors (e.g., tramadol), indoleamine uptake inhibitors (e.g., fluoxetine, paroxetine), narcotic analgesics (e.g., morphine), normarcotic analgesics (e.g., buprenorphine, axomadol), GABA receptor agonists, GABA uptake inhibitors (e.g., tiagabine), α₂ receptor agonists (e.g., clonidine), local analgesics (e.g., capsaicin), protein kinase C inhibitors (e.g., LY-333531), antianxiety drugs (e.g., benzodiazepines), phosphodiesterase inhibitors (e.g., sildenafil), dopamine receptor agonists (e.g., apomorphine), dopamine receptor antagonists (e.g., haloperidol), serotonin receptor agonists (e.g., tandospirone citrate, sumatryptan, tegaserod), serotonin receptor antagonists (e.g., cyproheptadine hydrochloride, ondansetron), serotonin uptake inhibitors (e.g., fluvoxamine maleate, fluoxetine, paroxetine), sleep-inducing drugs (e.g., triazolam, zolpidem), hypnotics (e.g., ramelteon), anticholinergic agents, α₁ receptor blocking agents (e.g., tamsulosin, urapidil, naftopidil), muscle relaxants (e.g., baclofen etc.), potassium channel openers (e.g., nicorandil), calcium channel blocking agents (e.g., nifedipine), chloride channel openers (e.g., lubiprostone), agents for preventing and/or treating Alzheimer's disease (e.g., donepezil, rivastigmine, galanthamine), agents for treating Parkinson's disease (e.g., L-dopa), agents for preventing and/or treating multiple sclerosis (e.g., interferon β-1a), histamine H₁ receptor inhibitors (e.g., promethazine hydrochloride), proton pump inhibitors (e.g., lansoprazole, omeprazole), antithrombotic agents (e.g., aspirin, cilostazol), NK-2 receptor antagonists, NK-3 receptor antagonists (e.g., talnetant), agents of treating HIV infection (saquinavir, zidovudine, lamivudine, nevirapine), agents of treating chronic obstructive pulmonary diseases (salmeterol, thiotropium bromide, cilomilast), diuretics (e.g., furosemide), antidiuretics (e.g., vasopressin V2 receptor agonist) and the like.

Anticholinergic agents include, for example, atropine, scopolamine, homatropine, tropicamide, cyclopentolate, but ylscopolamine bromide, propantheline bromide, methylbenactyzium bromide, mepenzolate bromide, flavoxate, pirenzepine, ipratropium bromide, trihexyphenidyl, oxybutynin, propiverine, darifenacin, tolterodine, solifenacin, temiverine, trospium chloride and a salt thereof (e.g., atropine sulfate, scopolamine hydrobromide, homatropine hydrobromide, cyclopentolate hydrochloride, flavoxate hydrochloride, pirenzepine hydrochloride, trihexyphenidyl hydrochloride, oxybutynin chloride, tolterodine tartrate, solifenacin succinate etc.), preferably, oxybutynin, propiverine, darifenacin, tolterodine, solifenacin, temiverine, trospium chloride and a salt thereof (e.g., oxybutynin chloride, tolterodine tartrate, solifenacin succinate etc.). In addition, acetylcholinesterase inhibitors (e.g., distigmine etc.) and the like can be used.

NK-2 receptor antagonists include, for example, a piperidine derivative such as GR159897, GR149861, SR48968 (saredutant), SR144190, YM35375, YM38336, ZD7944, L-743986, MDL105212A, ZD6021, MDL105172A, SCH205528, SCH62373, R-113281 etc., a perhydroisoindole derivative such as RPR-106145 etc., a quinoline derivative such as SB-414240 etc., a pyrrolopyrimidine derivative such as ZM-253270 etc., a pseudopeptide derivative such as MEN11420 (nepadutant), SCH217048, L-659877, PD-147714 (CAM-2291), MEN10376, S16474 etc., and others such as GR100679, DNK333, GR94800, UK-224671, MEN10376, MEN10627, or a salt thereof, and the like.

The pharmaceutical composition comprising a mixture or combination of the compound of the present invention and the concomitant drugs may be formulated into

(1) a single formulation as a pharmaceutical composition containing the compound of the present invention and the concomitant drugs, or

(2) a formulation comprising the compound of the present invention and the concomitant drugs which are separately formulated. Hereinafter, it is generally briefly referred to as the combination preparation of the present invention.

The combination preparation of the present invention can be formulated by mixing the compound of the present invention and active ingredients of the concomitant drugs separately or at the same time as itself or with pharmaceutically acceptable carriers in the same manner as in the method of producing the pharmaceutical preparation comprising the compound of the present invention.

The daily dose of the combination preparation of the present invention varies depending on the severity of symptoms, age, sex, body weight and sensitivity of the subject to be administered, time and interval of administration, property, formulation and kinds of pharmaceutical preparation, kinds of active ingredients etc., and is not particularly limited. While the dose of the compound of the present invention is not particularly limited as long as the dose does not problematically pose side effects, the daily dosage of the compound of the present invention is generally about 0.005 to 100 mg, preferably about 0.05 to 50 mg, and more preferably about 0.2 to 30 mg, per 1 kg body weight of a mammal generally by oral administration, which is generally administered in 1 to 3 portions a day.

The dose of the compound or a combination preparation of the present invention can be set for any amount as long as it does not cause problematic side effects. The daily dose of the compound or combination preparation of the present invention varies depending on the severity of symptoms, age, sex, body weight and sensitivity of the subject to be administered, time and interval of administration, property, formulation and kinds of pharmaceutical preparation, kinds of active ingredients etc., and is not particularly limited. The amount of the active ingredient is generally about 0.001 to 2000 mg, preferably about 0.01 to 500 mg, more preferably about 0.1 to 100 mg, per 1 kg body weight of a mammal by, for example, oral administration, which is generally administered in 1 to 4 portions a day.

In administering the combination preparation of the present invention, the compound of the present invention and the concomitant drugs may be administered at the same time or, the concomitant drugs may be administered before administering the compound of the present invention, and vice versa. In case of staggered administration, the time interval varies depending on the active ingredients to be administered, a formulation and an administration route. For example, if the concomitant drugs are administered first, the compound of the present invention may be administered 1 min. to 3 days, preferably 10 min. to 1 day, more preferably 15 min. to 1 hr after administering the concomitant drugs. If the compound of the present invention is administered first, the concomitant drugs may be administered 1 min. to 1 day, preferably 10 min. to 6 hrs., more preferably 15 min. to 1 hr after administering the compound of the present invention.

A preferable administration method of a daily dose includes, for example, oral administration of about 0.001 to 200 mg/kg of a concomitant drug formulated for oral administration, and about 15 min. later, oral administration of about 0.005 to 100 mg/kg of the compound of the present invention formulated for oral administration.

While the content of the compound of the present invention in the whole combination preparation in the present invention varies depending on the form of the preparation, it is generally about 0.01 to 100 wt %, preferably about 0.1 to 50 wt %, more preferably about 0.5 to 20 wt %, of the preparation as a whole.

### EXAMPLES

The present invention is further described in detail in with reference to Reference Examples, Examples, Preparative Examples and Experimental Examples which are not intended to restrict the invention and may be modified without departing from the scope of the invention.

Elution in the column chromatography in the following Reference Examples and Examples was conducted under observation by TLC (thin layer chromatography), unless otherwise specifically indicated. In the TLC observation, 60F254, TLC plates, produced by Merck & Co., Inc. was used, and the solvent employed as an elution solvent in the column chromatography was used as an eluent. For the detection, a UV detector was used. As silica gel for the column chromatography, Silica Gel 60 (70 to 230 mesh) produced by Merck & Co., Inc. was used. The “room temperature” referred herein means temperature generally from about 10° C. to 35° C. For drying extract, sodium sulfate or magnesium sulfate was used.

The abbreviations in Examples and Reference Examples mean the following.
LC: liquid chromatography
MS: mass spectrometry
ESI: electrospray ionization
FAB: fast atom bombardment
M: molecular ion peak
NMR: nuclear magnetic resonance
Hz: hertz
J: coupling constant
m: multiplet
q: quartet
t: triplet
d: doublet
S: singlet
br: broad
dt: double triplet
brs: broad singlet
^{t}Bu: tert-butyl group
Boc: tert-butoxycarbonyl group
Rf: retardation factor
Rt: retention time
N: normal concentration
MPa: megapascal
DMF: N,N-dimethylformamide
THF: tetrahydrofuran
DMSO: dimethyl sulfoxide
MeOH: methanol
IPE: diisopropyl ether
HOBt•H₂O: 1-hydroxybenzotriazole monohydrate
WSC•HCl: 1-ethyl-3-(dimethylaminopropyl)carbodiimide hydrochloride
Boc₂O: di-tert-butyl bicarbonate

LC-MS in Examples and Reference Examples was measured under the following conditions.

Analysis by LC-MS

Instrument: Waters LC-MS system

HPLC system: Agilent HP1100

MS system: Micromass ZMD

HPLC conditions
Column: CAPCELL PAK C18UG120, S-3 μm, 1.5×35 mm (Shiseido)
Solvents: Solution A; water containing 0.05% trifluoroacetic acid, Solution B; acetonitrile containing 0.05% trifluoroacetic acid
Gradient cycles: 0.00 min. (Solution A/Solution B=90/10), 2.00 min. (Solution A/Solution B=5/95), 2.75 min. (Solution A/Solution B=5/95), 2.76 min. (Solution A/Solution B=90/10), 3.60 min. (Solution A/Solution B=90/10)
Injection volume: 2 μL, Flow rate: 0.5 mL/min, Detection method: UV 220 nm
MS conditions
Ionization method: ESI
   Analysis by LC
Instrument: Shimadzu Corporation CLASS-VP system
HPLC conditions
Column: Inertsil ODS-2, CAPCELL PAK C18UG120, 5 μm, 4.6×150 mm (GL Sciences Inc.)
Solvents: Solution A; water containing 0.1% trifluoroacetic acid, Solution B; acetonitrile containing 0.1% trifluoroacetic acid
Gradient cycles: 0.00 min. (Solution A/Solution B=70/30), 15.00 min. (Solution A/Solution B=15/85), 15.01 min. (Solution A/Solution B=5/95), 20.00 min. (Solution A/Solution B=5/95), 20.01 min. (Solution A/Solution B=70/30), 25.00 min. (Solution A/Solution B=70/30)
Injection volume: 10 μL, Flow rate: 1.0 mL/min, Detection method: UV 220 nm

Purification by preparative HPLC in Examples and Reference Examples was carried out under the following conditions.
Instrument: High Throughput Purification System, Gilson Company, Inc.
Column: YMC CombiPrep ODS-AS-5 μm, 50×20 mm
Solvents: Solution A; water containing 0.1% trifluoroacetic acid, Solution B; 0.1% trifluoroacetic acid-containing acetonitrile
Gradient cycle: 0.00 minute (Solution A/Solution B=95/5), 1.00 minute (Solution A/Solution B=95/5), 5.20 min. (Solution A/Solution B=5/95), 6.40 min. (Solution A/Solution B=5/95), 6.50 min. (Solution A/Solution B=95/5), 6.60 min. (Solution A/Solution B=95/5)
Flow rate: 25 ml/min, Detection method: UV 220 nm
   HPLC Conditions (Measurement of Chemical Purity and Diastereomer Excess of Examples 23 and 24)
Column: YMC ODS PAK A-302 4.6 mmID×150 mm
Solvent: 50 mM potassium dihydrogenphosphate/acetonitrile=80/20
Injection volume: 20 μL
Flow rate: 1.0 mL/min
Detection method: UV 220 nm
   Chiral HPLC Conditions (Measurement of Enantiomer Excess of Examples 23 and 24)
Column: CHIRALCEL OD-RH 4.6 mmID×150 mm
Solvent: 50 mM potassium dihydrogenphosphate (pH 8.0)/acetonitrile=85/15
Injection volume: 20 μL
Flow rate: 0.3 mL/min
Temperature: 40° C.
Detection method: UV 220 nm
   HPLC Conditions (Measurement of Chemical Purity and Diastereomer Excess of Example 25)
Column: YMC ODS PAK A-302 4.6 mmID×150 mm
Solvent: 50 mM potassium dihydrogenphosphate/acetonitrile=50/50
Injection volume: 20 μL
Flow rate: 1.0 mL/min
Detection method: UV 220 nm
   Chiral HPLC Conditions (Measurement of Enantiomer Excess of Example 25)
Column: CHIRALPAK AD 4.6 mmID×150 mm
Solvent: hexane/2-propanol=50/50
Flow rate: 0.5 mL/min
Temperature: 25° C.
Detection method: UV 220 nm

The powder X-ray diffraction in the Examples and Reference Examples was measured under the following conditions.
Measurement device: Rigaku Corporation RINT Ultima⁺ 2100
Radiation source: Cu-K_{α} ray (λ=1.5418 Å)
Tube voltage: 40 kV
Tube current: 50 mA
Scanning rate: 6°/min
Angle of diffraction (2θ): 2 to 35°

### Reference Example 1

### N-{2-[(3R*,4S*)-4-({2-methoxy-5-[5-(trifluoromethyl)-1H-tetrazol-1-yl]benzyl}amino)-3-phenylpiperidin-1-yl]-1-methyl-2-oxoethyl}acetamide

To a solution of (3R*,4S*)-N-{2-methoxy-5-[5-(trifluoromethyl)-1H-tetrazol-1-yl]benzyl}-3-phenylpiperidine-4-amine dihydrochloride (0.20 g) (synthesized by a known method (WO03/101964 A1)), Et₃N (0.081 g) and N-acetyl-DL-alanine (0.079 g) in DMF (5 mL) were added WSC•HCl (0.12 g) and HOBt•H₂O (0.092 g), and the mixture was stirred at room temperature for 14 hrs. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 50→100% ethyl acetate/hexane) to give the title compound as a white amorphous solid (0.22 g, 99%).

MS(ESI+): 546(M+H)

In the same manner as in Reference Example 1 and using (3R*,4S*)-N-{2-methoxy-5-[5-(trifluoromethyl)-1H-tetrazol-1-yl]benzyl}-3-phenylpiperidine-4-amine dihydrochloride and the corresponding carboxylic acid, the compounds of Reference Examples 2 to 11 were obtained (compounds obtained in Reference Examples 3, 5, 6 and 9 to 11 were each treated with 1 equivalent of hydrogen chloride/ethyl acetate and isolated as monohydrochloride).

### Reference Example 2

### N-{2-[(3R*,4S*)-4-({2-methoxy-5-[5-(trifluoromethyl)-1H-tetrazol-1-yl]benzyl}amino)-3-phenylpiperidin-1-yl]-1,1-dimethyl-2-oxoethyl}acetamide

MS(ESI+): 560(M+H)

### Reference Example 3

### 2-hydroxy-N-{2-[(3R*,4S*)-4-({2-methoxy-5-[5-(trifluoromethyl)-1H-tetrazol-1-yl]benzyl}amino)-3-phenylpiperidin-1-yl]-2-oxoethyl}acetamide monohydrochloride

MS(ESI+): 548(M−HCl+H)

### Reference Example 4

### N-{2-[(3R,4S*)-4-({2-methoxy-5-[5-(trifluoromethyl)-1H-tetrazol-1-yl]benzyl}amino)-3-phenylpiperidin-1-yl]-2-oxoethyl}urea

MS(ESI+): 533 (M+H)

### Reference Example 5

### N-{2-[(3R*,4S*)-4-({2-methoxy-5-[5-(trifluoromethyl)-1H-tetrazol-1-yl]benzyl}amino)-3-phenylpiperidin-1-yl]-2-oxoethyl)-N-methylformamide monohydrochloride

MS(ESI+): 532 (M−HCl+H)

### Reference Example 6

### N-{4-[(3R*,4S*)-4-({2-methoxy-5-[5-(trifluoromethyl)-1H-tetrazol-1-yl]benzyl}amino)-3-phenylpiperidin-1-yl]-4-oxobutyl}acetamide monohydrochloride

MS(ESI+): 560(M−HCl+H)

### Reference Example 7

### 4-[(3R*,4S*)-4-({2-methoxy-5-[5-(trifluoromethyl)-1H-tetrazol-1-yl]benzyl}amino)-3-phenylpiperidin-1-yl]-4-oxobutanamide

MS(ESI+): 532 (M+H)

### Reference Example 8

### (3R*,4S*)-N-{2-methoxy-5-[5-(trifluoromethyl)-1H-tetrazol-1-yl]benzyl)-1-[(methylthio)acetyl]-3-phenylpiperidine-4-amine

MS(ESI+): 521 (M+H)

### Reference Example 9

### 5-{[(3R*,4S*)-4-({2-methoxy-5-[5-(trifluoromethyl)-1H-tetrazol-1-yl]benzyl}amino)-3-phenylpiperidin-1-yl]carbonyl}-1-methylpyrrolidin-2-one monohydrochloride

MS(ESI+): 558(M−HCl+H)

### Reference Example 10

### 4-{[(3R,4S*)-4-({2-methoxy-5-[5-(trifluoromethyl)-1H-tetrazol-1-yl]benzyl}amino)-3-phenylpiperidin-1-yl]carbonyl}-2,6-piperidinedione monohydrochloride

MS(ESI+): 572(M−HCl+H)

### Reference Example 11

### 6-{[(3R*,4S*)-4-({2-methoxy-5-[5-(trifluoromethyl)-1H-tetrazol-1-yl]benzyl}amino)-3-phenylpiperidin-1-yl]carbonyl}-1,2,3,4-tetrahydropyrimidine-2,4-dione monohydrochloride

MS(ESI+): 571 (M−HCl+H)

### Reference Example 12

### (3R*,4S*)-N-(2-methoxy-5-[5-(trifluoromethyl)-1H-tetrazol-1-yl]benzyl}-1-[(methylsulfinyl)acetyl]-3-phenylpiperidine-4-amine monohydrochloride

To a solution of the compound (0.23 g) obtained in Reference Example 8 in CH₂Cl₂ (5 mL) was added m-chloroperbenzoic acid (0.084 g) at 0° C., and the mixture was stirred for 1 hr. The reaction mixture was poured into a saturated solution of sodium hydrogencarbonate, and the product was extracted with ethyl acetate. The organic layer was washed with brine, and dried, and the solvent was evaporated under reduced pressure. The obtained residue was purified by silica gel column chromatography (solvent gradient; 0→10% methanol/ethyl acetate) to give a colorless oil (0.12 g). The obtained oil (0.12 g) was treated with 1 equivalent of 4N hydrogen chloride/ethyl acetate to give the title compound as a white powder (0.12 g, 48%).

MS(ESI+):537(M−HCl+H)

### Reference Examples 13-14

### tert-butyl (3R*,4S*)-4-({2-(cyclopropyloxy)-5-[5-(trifluoromethyl)-1H-tetrazol-1-yl]benzyl}amino)-3-phenylpiperidine-1-carboxylate (Reference Example 13)

### tert-butyl (3R*,4R*)-4-({2-(cyclopropyloxy)-5-[5-(trifluoromethyl)-1H-tetrazol-1-yl]benzyl}amino)-3-phenylpiperidine-1-carboxylate (Reference Example 14)

To a solution of tert-butyl 4-amino-3-phenylpiperidine-1-carboxylate (cis/trans mixture) (0.60 g) (synthesized by a known method (WO03/101964 A1)) and 2-(cyclopropyloxy)-5-[5-(trifluoromethyl)-1H-tetrazol-1-yl]benzaldehyde (0.54 g) (synthesized by a known method (WO99/24423)) in acetic acid (0.10 mL) and CH₂Cl₂ (10 mL) was added NABH(OAc)₃ (0.69 g), and the mixture was stirred at room temperature for 1 hr. An additional portion of NaBH(OAc)₃ (0.60 g) was added, and the mixture was stirred at room temperature for 1 hr. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 10→50% ethyl acetate/hexane) to give the compound of Reference Example 13 ((3R*,4S*)-form, 0.56 g, 55%) as a white amorphous solid, and the compound of Reference Example 14 ((3R*,4R*)-form, 0.44 g, 44%) as a colorless oil.

Compound ((3R*,4S*)-form) of Reference Example 13: Rf=0.6 (hexane:ethyl acetate=1:2)

MS(ESI+): 503 (M−^{t}Bu+2H)

Compound ((3R*,4R*)-form) of Reference Example 14: Rf=0.4 (hexane:ethyl acetate=1:2)

MS(ESI+): 503 (M−^{t}Bu+2H)

### Reference Example 15

### (3R*,4S*)-N-(2-(cyclopropyloxy)-5-[5-(trifluoromethyl)-1H-tetrazol-1-yl]benzyl}-3-phenylpiperidine-4-amine dihydrochloride

To a solution of the compound (0.55 g) obtained in Reference Example 13 in methanol (10 mL) was added 4N hydrogen chloride/ethyl acetate (0.98 mL), and the mixture was stirred at 50° C. for 2 hrs. The reaction mixture was concentrated under reduced pressure to give the title compound as a white amorphous solid (0.45 g).

MS(ESI+):459(M−2HCl+H)

### Reference Example 16

### (3R*,4S*)-1-[(1-acetylpiperidin-4-yl)carbonyl]-N-(2-(cyclopropyloxy)-5-[5-(trifluoromethyl)-1H-tetrazol-1-yl]benzyl}-3-phenylpiperidine-4-amine monohydrochloride

To a solution of the compound (0.20 g) obtained in Reference Example 15 and 1-acetylpiperidine-4-carboxylic acid (0.061 g) in DMF (5.0 mL) were added WSC•HCl (0.11 g) and HOBt•H₂O (0.086 g), and the mixture was stirred at room temperature for 14 hrs. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by preparative HPLC to give a colorless oil (0.21 g). The obtained oil (0.21 g) was treated with 1 equivalent of 4N hydrogen chloride/ethyl acetate to give the title compound as a white powder (0.17 g, 70%).

MS(ESI+): 612 (M−HCl+H)

Elemental analysis: C₃₁H₃₆F₃N₇O₃·2.5H₂O

Found C, 53.72; H, 6.11; N, 14.14.

Calculated C, 53.68; H, 5.89; N, 14.28.

### Reference Examples 17-18

### tert-butyl (3R,4S*)-4-({2-ethoxy-5-[5-(trifluoromethyl)-1H-tetrazol-1-yl]benzyl}amino)-3-phenylpiperidine-1-carboxylate (Reference Example 17)

### tert-butyl (3R*,4R*)-4-({2-ethoxy-5-[5-(trifluoromethyl)-1H-tetrazol-1-yl]benzyl}amino)-3-phenylpiperidine-1-carboxylate (Reference Example 18)

To a solution of tert-butyl 4-amino-3-phenylpiperidine-1-carboxylate (cis/trans mixture) (1.0 g) (synthesized by a known method (WO03/101964 A1)) and 2-ethoxy-5-[5-(trifluoromethyl)-1H-tetrazol-1-yl]benzaldehyde (0.90 g) (synthesized by a known method (WO03/101964 A1)) in acetic acid (0.12 mL) and CH₂Cl₂ (12 mL) was added NABH(OAc)₃ (1.2 g), and the mixture was stirred at room temperature for 1 hr. An additional portion of NaBH(OAc)₃ (1.2 g) was added, and the mixture was stirred at room temperature for 0.1 hr. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 20→50% ethyl acetate/hexane) to give the compound of Reference Example 17 as a white amorphous solid ((3R,4S*)-form, 1.0 g, 51%), and the compound of Reference Example 18 as a colorless oil ((3R*,4R*)-form, 0.80 g, 48%).

compound ((3R*,4S*)-form) of Reference Example 17: Rf=0.8 (hexane:ethyl acetate=1:1)

MS(ESI+): 491 (M−^{t}Bu+2H)

compound ((3R*,4R*)-form) of Reference Example 18: Rf=0.4 (hexane:ethyl acetate=1:1)

MS(ESI+): 491(M−^{t}Bu+2H)

### Reference Example 19

### (3R*,4S*)-N-{2-ethoxy-5-[5-(trifluoromethyl)-1H-tetrazol-1-yl]benzyl}-3-phenylpiperidine-4-amine dihydrochloride

To a solution of (3R*,4S*)-form (1.13 g) obtained in Reference Example 17 in methanol (15 mL) was added 4N hydrogen chloride/ethyl acetate (2.1 mL), and the mixture was stirred at 50° C. for 2 hrs. The reaction mixture was concentrated under reduced pressure to give the title compound as a white amorphous solid (1.1 g).

MS(ESI+): 447(M−2HCl+H)

### Reference Example 20

### (3R*,4S*)-1-[(1-acetylpiperidin-4-yl)carbonyl]-N-{2-ethoxy-5-[5-(trifluoromethyl)-1H-tetrazol-1-yl]benzyl}-3-phenylpiperidine-4-amine monohydrochloride

To a solution of the compound (0.20 g) obtained in Reference Example 19 and 1-acetylpiperidine-4-carboxylic acid (0.062 g) in DMF (5.0 mL) were added WSC•HCl (0.11 g) and HOBt•H₂O (0.088 g), and the mixture was stirred at room temperature for 14 hrs. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by preparative HPLC to give colorless oil (0.20 g). The obtained oil (0.20 g) was treated with 1 equivalent of 4N hydrogen chloride/ethyl acetate to give the title compound as a white powder (0.18 g, 74%).

MS(ESI+): 600(M−HCl+H)

Elemental analysis: C₃₀H₃₆F₃N₇O₃·2.25H₂O

Found C, 53.25; H, 6.18; N, 14.49.

Calculated C, 53.22; H, 6.05; N, 14.57.

### Reference Example 21

### tert-butyl (3R*,4S)-4-({2-hydroxy-5-[5-(trifluoromethyl)-1H-tetrazol-1-yl]benzyl}amino)-3-phenylpiperidine-1-carboxylate

To a solution tert-butyl (3R*,4S*)-4-amino-3-phenylpiperidine-1-carboxylate (0.61 g) (synthesized by a known method (WO03/101964 A1)) and 2-hydroxy-5-[5-(trifluoromethyl)-1H-tetrazol-1-yl]benzaldehyde (0.52 g) (synthesized by a known method (WO95/08549 A1)) in acetic acid (0.050 mL) and CH₂Cl₂ (10 mL) was added NABH(OAc)₃ (0.64 g), and the mixture was stirred at room temperature for 1 hr. An additional portion of NaBH(OAc)₃ (0.60 g) was added, and the mixture was stirred at room temperature for 1 hr. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 10→20% ethyl acetate/hexane) to give the title compound as a white amorphous solid (1.03 g, 99%).

MS(ESI+): 463(M−^{t}Bu+2H)

### Reference Example 22

### 2-({[(3R*,4S*)-3-phenylpiperidin-4-yl]amino}methyl)-4-[5-(trifluoromethyl)-1H-tetrazol-1-yl]phenol dihydrochloride

To a solution of the compound (1.0 g) obtained in Reference Example 21 in methanol (10 mL) was added 4N hydrogen chloride/ethyl acetate (2.0 mL), and the mixture was stirred at 50° C. for 2 hrs. The reaction mixture was concentrated under reduced pressure to give the title compound as colorless crystals.

MS(ESI+):419 (M−2HCl+H)

### Reference Example 23

### 2-[({(3R*,4S)-1-[(1-acetylpiperidin-4-yl)carbonyl]-3-phenylpiperidin-4-yl}amino)methyl]-4-[5-(trifluoromethyl)-1H-tetrazol-1-yl]phenol monohydrochloride

To a solution of the compound (0.20 g) obtained in Reference Example 22 and 1-acetylpiperidine-4-carboxylic acid (0.070 g) in DMF (5.0 mL) were added WSC•HCl (0.12 g) and HOBt•H₂O (0.095 g), and the mixture was stirred at room temperature for 14 hrs. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by preparative HPLC to give colorless oil (0.17 g). The obtained oil (0.17 g) was treated with 1 equivalent of 4N hydrogen chloride/ethyl acetate to give the title compound as a white amorphous solid (0.10 g, 40%).

MS(ESI+): 572 (M−HCl+H)

### Reference Example 24

### N-{2-[(3R*,4S*)-4-({2-hydroxy-5-[5-(trifluoromethyl)-1H-tetrazol-1-yl]benzyl}amino)-3-phenylpiperidin-1-yl]-2-oxoethyl}acetamide monohydrochloride

In the same manner as in Reference Example 23 and using the compound obtained in Reference Example 22 and N-acetylglycine, the title compound was obtained as a white amorphous solid.

MS(ESI+): 518(M−HCl+H)

### Reference Example 25

### tert-butyl (3R*,4R*)-4-({2-methoxy-5-[5-(trifluoromethyl)-1H-tetrazol-1-yl]benzyl}amino)-3-phenylpiperidine-1-carboxylate

To a solution of tert-butyl (i)-4-amino-3-phenylpiperidine-1-carboxylate (cis/trans, racemic mixture) (1.5 g) (synthesized by a known method (WO03/101964 A1)) and 2-methoxy-5-[5-(trifluoromethyl)-1H-tetrazol-1-yl]benzaldehyde (1.3 g) (synthesized by a known method (J. Labelled Cpd. Radiopharm., vol. 43, pp. 29-45)) in acetic acid (0.1 mL) and CH₂Cl₂ (20 mL) was added NaBH(OAc)₃ (1.7 g), and the mixture was stirred at room temperature for 1 hr. An additional portion of NABH(OAc)₃ (1.7 g) was added, and the mixture was stirred at room temperature for 1 hr. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 5→50% ethyl acetate/hexane) to give a (3R*,4S*)-isomer (1.9 g) of the title compound and the title compound (0.60 g) as a colorless oil.

(3R*,4S*)-form: Rf=0.4 (hexane:ethyl acetate=1:1)

MS(ESI+): 477 (M−^{t}Bu+2H)

compound ((3R*,4R*)-form) of Reference Example 25: Rf=0.2 (hexane:ethyl acetate=1:1)

MS(ESI+): 477(M−^{t}Bu+2H)

### Reference Example 26

### (3R*,4R*)-N-{2-methoxy-5-[5-(trifluoromethyl)-1H-tetrazol-1-yl]benzyl}-3-phenylpiperidine-4-amine dihydrochloride

To a solution of the compound (0.60 g) obtained in Reference Example 25 in methanol (5.0 mL) was added 4N hydrogen chloride/ethyl acetate (1.2 mL), and the mixture was stirred at 50° C. for 2 hrs. The reaction mixture was concentrated under reduced pressure to give the title compound as a white amorphous solid (0.63 g).

MS(ESI+): 433(M−2HCl+H)

### Reference Example 27

### (3R*,4R*)-1-[(1-acetyl-4-piperidinyl)carbonyl]-N-{2-methoxy-5-[5-(trifluoromethyl)-1H-tetrazol-1-yl]benzyl}-3-phenylpiperidine-4-amine

To a solution of the compound (0.92 g) obtained in Reference Example 26 and 1-acetylpiperidine-4-carboxylic acid (0.30 g) in DMF (5 mL) were added WSC•HCl (0.52 g) and HOBt•H₂O (0.42 g), and the mixture was stirred at room temperature for 14 hrs. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 50→100% ethyl acetate/hexane) to give the title compound as a white amorphous solid (0.80 g, 75%).

MS(ESI+): 586(M+H)

### Reference Example 28

### N-{2-[(3R*,4R*)-4-({2-methoxy-5-[5-(trifluoromethyl)-1H-tetrazol-1-yl]benzyl}amino)-3-phenylpiperidin-1-yl]-2-oxoethyl}acetamide

To a solution of the compound (1.3 g) obtained in Reference Example 26 and N-acetylglycine (0.45 g) in DMF (2.5 mL) were added WSC•HCl (0.74 g) and HOBt•H₂O (0.59 g), and the mixture was stirred at room temperature for 14 hrs. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 50→100% ethyl acetate/hexane) to give the title compound as a white amorphous solid (1.2 g, 88%).

MS(ESI+): 532 (M+H)

The compounds described in Reference Examples 1-28 are as follows (Tables 1-3).

TABLE 1

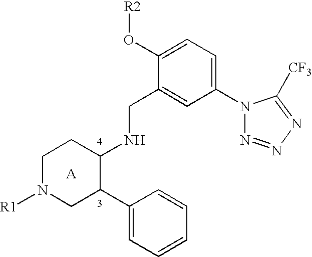

| Ref. Ex. | | | | | |
|---|---|---|---|---|---|
| No. | R1 | R2 | 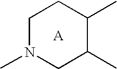 | additives | MS (ESI) |
| 1 | 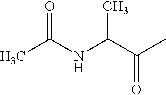 | CH₃ | 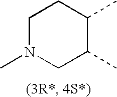 | | 546 (M + H) |
| 2 | 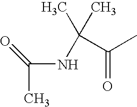 | CH₃ | 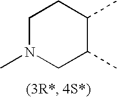 | | 560 (M + H) |
| 3 | 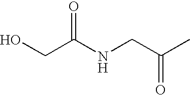 | CH₃ | 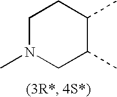 | HCl | 548 (M − HCl + H) |
| 4 | 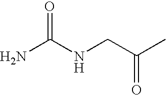 | CH₃ | 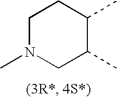 | | 533 (M + H) |
| 5 | 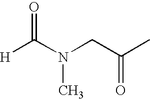 | CH₃ | 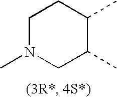 | HCl | 532 (M − HCl + H) |
| 6 | 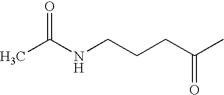 | CH₃ | 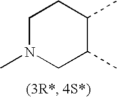 | HCl | 560 (M − HCl + H) |
| 7 | 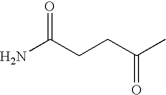 | CH₃ | 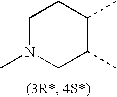 | | 532 (M + H) |
| 8 | 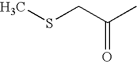 | CH₃ | 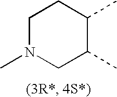 | | 521 (M + H) |
| 9 | 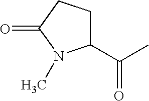 | CH₃ | 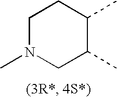 | HCl | 558 (M − HCl + H) |

TABLE 2

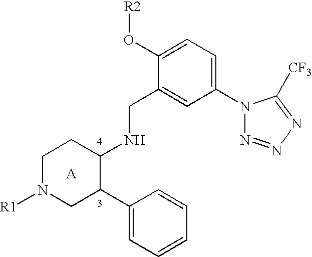

| Ref. Ex. | | | | | |
|---|---|---|---|---|---|
| No. | R1 | R2 | 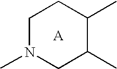 | additives | MS (ESI) |
| 10 | 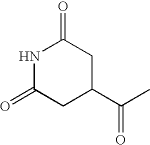 | CH₃ | 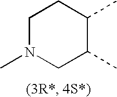 | HCl | 572 (M − HCl + H) |
| 11 | 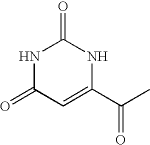 | CH₃ | 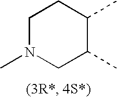 | HCl | 571 (M − HCl + H) |
| 12 | 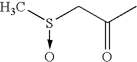 | CH₃ | 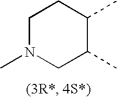 | HCl | 537 (M − HCl + H) |
| 13 | 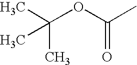 | 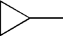 | 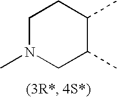 | | 503 (M − ^{t}Bu + 2H) |
| 14 | 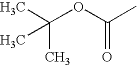 | 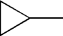 | 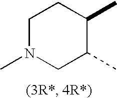 | | 503 (M − ^{t}Bu + 2H) |
| 15 | H | 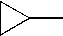 | 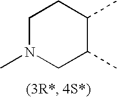 | 2HCl | 459 (M − HCl + H) |
| 16 | 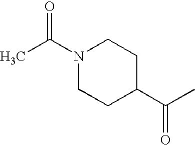 | 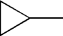 | 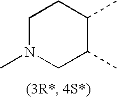 | HCl | 612 (M − HCl + H) |
| 17 | 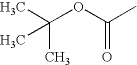 | C₂H₅ | 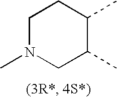 | | 491 (M − ^{t}Bu + 2H) |
| 18 | 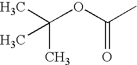 | C₂H₅ | 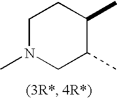 | | 491 (M − ^{t}Bu + 2H) |

TABLE 3

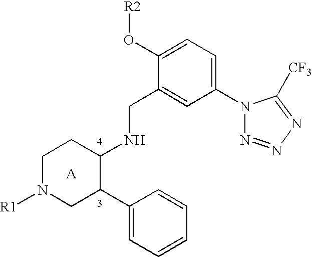

| Ref. Ex. | | | | | |
|---|---|---|---|---|---|
| No. | R1 | R2 | 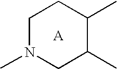 | additives | MS (ESI) |
| 19 | H | C₂H₅ | 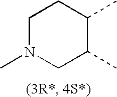 | 2HCl | 447 (M − 2HCl + H) |
| 20 | 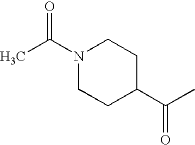 | C₂H₅ | 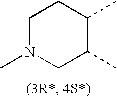 | HCl | 600 (M − HCl + H) |
| 21 | 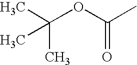 | H | 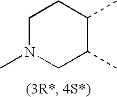 | | 463 (M − ^{t}Bu + 2H) |
| 22 | H | H | 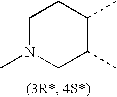 | 2HCl | 419 (M − 2HCl + H) |
| 23 | 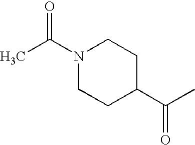 | H | 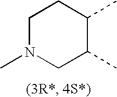 | HCl | 572 (M − HCl + H) |
| 24 | 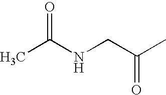 | H | 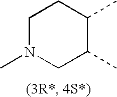 | HCl | 518 (M − HCl + H) |
| 25 | 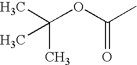 | CH₃ | 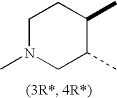 | | 477 (M − ^{t}Bu + 2H) |
| 26 | H | CH₃ | 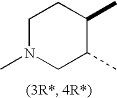 | 2HCl | 433 (M − 2HCl + H) |
| 27 | 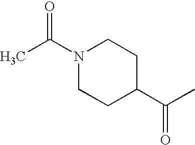 | CH₃ | 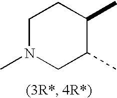 | | 586 (M + H) |
| 28 | 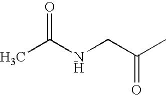 | CH₃ | 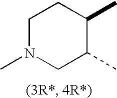 | | 532 (M + H) |

### Examples 1-2

### (+)-(3R*,4R*)-1-[(1-acetyl-4-piperidinyl)carbonyl]-N-{2-methoxy-5-[5-(trifluoromethyl)-1H-tetrazol-1-yl]benzyl}-3-phenylpiperidine-4-amine monohydrochloride (Example 1)

### (−)-(3R,4R*)-1-[(1-acetyl-4-piperidinyl)carbonyl]-N-{2-methoxy-5-[5-(trifluoromethyl)-1H-tetrazol-1-yl]benzyl}-3-phenylpiperidine-4-amine monohydrochloride (Example 2)

The compound (0.75 g) obtained in Reference Example 27 was optically resolved by chiral HPLC, and the fractions were concentrated under reduced pressure. A white amorphous solid (0.28 g; [α]_{D}²⁵+13.0° (c 1.0, MeOH)) was obtained from a fraction having a shorter Rt, which was treated with 1 equivalent of 4N hydrogen chloride/ethyl acetate to give the compound (0.20 g) of Example 1.

MS(ESI+): 586 (M−HCl+H)

A white amorphous solid (0.048 g) was obtained from a fraction having a longer Rt, which was treated with 1 equivalent of 4N hydrogen chloride/ethyl acetate to give the compound (0.021 g) of Example 2 as a white amorphous solid.

MS(ESI+): 586 (M−HCl+H)

Chiral HPLC Conditions

Column: CHIRALPAK OJ 50 mmID×500 mmL
Solvent: hexane/ethanol=85/25
Flow rate: 80 mL/min
Temperature: 40° C.
Detection method: UV 220 nm

### Example 3

### tert-butyl (3R,4S)-4-({2-methoxy-5-[5-(trifluoromethyl)-1H-tetrazol-11-yl]benzyl}amino)-3-phenylpiperidine-1-carboxylate

To a solution of tert-butyl (3R,4S)-4-amino-3-phenylpiperidine-1-carboxylate (same as tert-butyl (+)-cis-4-amino-3-phenylpiperidine-1-carboxylate) (5.0 g) (synthesized by a known method (WO03/101964 A1)) and 2-methoxy-5-[5-(trifluoromethyl)-1H-tetrazol-1-yl]benzaldehyde (4.9 g) (synthesized by a known method (J. Labelled Cpd. Radiopharm., vol. 43, pp. 29-45)) in acetic acid (0.90 mL) and CH₂Cl₂ (90 mL) was added NABH(OAc)₃ (5.8 g), and the mixture was stirred at room temperature for 1 hr. An additional portion of NABH(OAc)₃ (4.0 g) was added, and the mixture was stirred at room temperature for 1 hr. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 5→50% ethyl acetate/hexane) to give the title compound as a white amorphous solid (8.8 g, 96%).

MS(ESI+): 477(M−^{t}Bu+2H)

### Example 4

### (3R,4S)-N-{2-methoxy-5-[5-(trifluoromethyl)-1H-tetrazol-1-yl]benzyl}-3-phenylpiperidine-4-amine monohydrochloride

To a solution of the compound (37.6 g) obtained in Example 3 in methanol (200 mL) was added 4N hydrogen chloride/ethyl acetate (70.6 mL), and the mixture was stirred at 50° C. for 3 hrs. The reaction mixture was concentrated under reduced pressure, and the residue was suspended in water (300 mL) and chloroform (200 mL). An 8N solution of sodium hydroxide (50 mL) was added, and the organic layer was separated. The aqueous layer was further extracted with chloroform (200 mL). The organic layer was dried, and the solvent was evaporated under reduced pressure. The obtained residue (free amine form, 30.5 g) was dissolved in ethyl acetate, and 1 equivalent of 4N hydrogen chloride/ethyl acetate (16.7 mL) was added. The precipitate was collected by filtration, and recrystallized from ethanol to give the title compound as colorless crystals (25.7 g, 78%).

MS(ESI+):433(M−HCl+H)

### Example 5

### {2-[(3R,4S)-4-({2-methoxy-5-[5-(trifluoromethyl)-1H-tetrazol-1-yl]benzyl}amino)-3-phenylpiperidin-1-yl]-2-oxoethyl}formamide

To a solution of the compound (0.20 g) obtained in Example 4, Et₃N (0.081 g) and N-formylglycine (0.062 g) in DMF (6 mL) were added WSC•HCl (0.12 g) and HOBt•H₂O (0.092 g), and the mixture was stirred at room temperature for 14 hrs. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 50→100% ethyl acetate/hexane) to give the title compound as a white amorphous solid (0.20 g, 97%).

MS(ESI+): 518(M+H)

In the same manner as in Example 5 and using the compound obtained in Example 4 and the corresponding carboxylic acid, the compounds of Examples 6-9 were obtained (these compounds were each treated with 1 equivalent of hydrogen chloride/ethyl acetate and isolated as monohydrochloride).

### Example 6

### (3R,4S)-1-acetyl-N-{2-methoxy-5-[5-(trifluoromethyl)-1H-tetrazol-1-yl]benzyl}-3-phenylpiperidine-4-amine monohydrochloride

MS(ESI+): 475 (M−HCl+H)

### Example 7

### 1-{2-[(3R,4S)-4-({2-methoxy-5-[5-(trifluoromethyl)-1H-tetrazol-1-yl]benzyl}amino)-3-phenylpiperidin-1-yl]-2-oxoethyl}pyrrolidine-2,5-dione monohydrochloride

MS(ESI+): 572(M−HCl+H)

### Example 8

### (3R,4S)-N-{2-methoxy-5-[5-(trifluoromethyl)-1H-tetrazol-1-yl]benzyl}-1-[(methylsulfonyl)acetyl]-3-phenylpiperidine-4-amine monohydrochloride

MS(ESI+): 553(M−HCl+H)

### Example 9

### N-{2-[(3R,4S)-4-({2-methoxy-5-[5-(trifluoromethyl)-1H-tetrazol-1-yl]benzyl}amino)-3-phenylpiperidin-1-yl]-2-oxoethyl}propanamide monohydrochloride

MS(ESI+): 546 (M−HCl+H)

### Example 10

### (3R,4S)-N-(2-methoxy-5-[5-(trifluoromethyl)-1H-tetrazol-1-yl]benzyl)-3-phenyl-1-(piperidin-4-ylcarbonyl)piperidine-4-amine dihydrochloride

(Step 1)

To a solution of the compound (0.25 g) obtained in Example 4, Et₃N (0.10 g) and Boc-isonipecotic acid (0.17 g) in DMF (6 mL) were added WSC•HCl (0.14 g) and HOBt•H₂O (0.12 g), and the mixture was stirred at room temperature for 18 hrs. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 50→100% ethyl acetate/hexane) to give a white amorphous solid (0.31 g).

(Step 2)

To a solution of the compound (0.42 g) obtained in step 1 in methanol (25 mL) was added 4N hydrogen chloride/ethyl acetate (0.65 mL), and the mixture was stirred at 50° C. for 10 hrs. The reaction mixture was concentrated under reduced pressure, and the residue was crystallized from methanol and IPE to give the title compound as colorless crystals (0.31 g, 88%).

MS(ESI+): 544 (M−2HCl+H)

### Example 11

### (3R,4S)-N-{2-methoxy-5-[5-(trifluoromethyl)-1H-tetrazol-1-yl]benzyl}-1-{[1-(methylsulfonyl)piperidin-4-yl]carbonyl}-3-phenylpiperidine-4-amine monohydrochloride

To a solution of the compound (0.25 g) obtained in Example 10 and Et₃N (0.083 g) in DMF (2.5 mL)-THF (2.5 mL) was added methanesulfonyl chloride (0.061 g) at −78° C., and the mixture was stirred at 0° C. for 30 min. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 10→50% ethyl acetate/hexane) to give a white amorphous solid (0.13 g). The obtained white amorphous solid (0.13 g) was treated with 4N hydrogen chloride/ethyl acetate (0.050 mL) to give the title compound as a white amorphous solid (0.12 g, 44%).

MS(ESI+): 622(M−HCl+H)

### Examples 12-13

### N-{1-hydroxy-2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide (shorter Rt: Example 12, longer Rt: Example 13)

(Step 1)

To a solution of the compound obtained in Example 4 (0.30 g), Et₃N (0.065 g) and (acetylamino)(hydroxy)acetic acid (0.11 g) synthesized by a known method (Journal of Organic Chemistry, vol. 55, pp. 4657-4663, 1990) in DMF (5 mL) were added WSC•HCl (0.18 g) and HOBt•H₂O (0.15 g), and the mixture was stirred at room temperature for 18 hrs. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 50→100% ethyl acetate/hexane) to give a white amorphous solid (0.21 g) as diastereomer mixture (1:1).

(Step 2)

The compound (0.50 g) obtained in Step 1 was subjected to diastereomer resolution by chiral HPLC, and the fractions were concentrated under reduced pressure. The compound of Example 12 was obtained as a white amorphous solid (0.088 g) from the fraction having a shorter Rt.

MS(ESI+): 548 (M+H)

In addition, the compound of Example 13 was obtained as a white amorphous solid from the fraction having a longer Rt.

MS(ESI+): 548(M+H)

Chiral HPLC Conditions

Column: CHIRALPAK AD 50 mmID×500 mmL
Solvent: hexane/2-propanol=70/30
Flow rate: 60 mL/min
Temperature: 25° C.
Detection method: UV 254 nm

### Example 14

### N-{2-[(3R,4S)-4-({2-hydroxy-5-[5-(trifluoromethyl)-1H-tetrazol-1-yl]benzyl}amino)-3-phenylpiperidin-1-yl]-2-oxoethyl}acetamide

(Step 1)

To a solution of tert-butyl (3R,4S)-4-amino-3-phenylpiperidine-1-carboxylate (same as tert-butyl (+)-cis-4-amino-3-phenylpiperidine-1-carboxylate)(3.0 g) (synthesized by a known method (WO03/101964 A1)) and 2-hydroxy-5-[5-(trifluoromethyl)-1H-tetrazol-1-yl]benzaldehyde (2.6 g) (synthesized by a known method (WO95/08549 A1)) in acetic acid (0.23 mL) and CH₂Cl₂ (45 mL) was added NABH(OAc)₃ (3.1 g), and the mixture was stirred at room temperature for 1 hr. An additional portion of NABH(OAc)₃ (3.1 g) was added, and the mixture was stirred at room temperature for 1 hr. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 10→20% ethyl acetate/hexane) to give white amorphous solid (5.1 g, 99%).

MS(ESI+): 463 (M−^{t}Bu+2H)

(Step 2)

To a solution of the compound (5.6 g) obtained in step 1 in methanol (30 mL) was added 4N hydrogen chloride/ethyl acetate (10 mL), and the mixture was stirred at 50° C. for 2 hrs. The reaction mixture was concentrated under reduced pressure to give colorless crystals (4.8 g, 99%).

MS(ESI+): 419(M−2HCl+H)

(Step 3)

To a solution of the compound (4.8 g) obtained in step 2 and N-acetylglycine (1.7 g) in DMF (30 mL) were added WSC•HCl (2.8 g) and HOBt•H₂O (2.3 g), and the mixture was stirred at room temperature for 14 hrs. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 33→100% ethyl acetate/hexane) to give the title compound as a white amorphous solid (3.2 g, 63%).

MS(ESI+): 518(M+H)

### Examples 15-16

### (+)-N-{2-[(3R*,4R*)-4-({2-methoxy-5-[5-(trifluoromethyl)-1H-tetrazol-1-yl]benzyl}amino)-3-phenylpiperidin-1-yl]-2-oxoethyl}acetamide monohydrochloride (Example 15)

### (−)-N-{2-[(3R*,4R*)-4-({2-methoxy-5-[5-(trifluoromethyl)-1H-tetrazol-1-yl]benzyl}amino)-3-phenylpiperidin-1-yl]-2-oxoethyl}acetamide monohydrochloride (Example 16)

The compound (0.94 g) obtained in Reference Example 28 was subjected to optical resolution by chiral HPLC, and the fractions were concentrated under reduced pressure. A white amorphous solid (0.46 g; [α]_{D}²⁵+13.6° (c 1.0, MeOH)) was obtained from the fraction having a shorter Rt, which was treated with 1 equivalent of 4N hydrogen chloride/ethyl acetate to give the compound of Example 15 (0.27 g).

MS(ESI+): 532 (M−HCl+H)

A white amorphous solid (0.47 g; [α]_{D}²⁵-12.0° (c 1.0, MeOH)) was obtained from the fraction having a longer Rt, which was treated with 1 equivalent of 4N hydrogen chloride/ethyl acetate to give the compound of Example 16 as a white amorphous solid (0.29 g).

MS(ESI+): 532 (M−HCl+H)

Chiral HPLC Conditions

Column: CHIRALPAK OD 50 mmID×500 mmL
Solvent: hexane/ethanol=80/20
Flow rate: 60 mL/min
Temperature: 30° C.
Detection method: UV 230 nm

### Example 17

### tert-butyl (3S,4R)-4-({2-methoxy-5-[5-(trifluoromethyl)-1H-tetrazol-1-yl]benzyl}amino)-3-phenylpiperidine-1-carboxylate

To a solution of tert-butyl (3S,4R)-4-amino-3-phenylpiperidine-1-carboxylate (same as tert-butyl (−)-cis-4-amino-3-phenylpiperidine-1-carboxylate) (0.55 g) (synthesized by a known method (WO03/101964 A1)) and 2-methoxy-5-[5-(trifluoromethyl)-1H-tetrazol-1-yl]benzaldehyde (0.54 g) (synthesized by a known method (J. Labelled Cpd. Radiopharm., vol. 43, pp. 29-45)) in acetic acid (0.10 mL) and CH₂Cl₂ (10 mL) was added NABH(OAc)₃ (0.64 g), and the mixture was stirred at room temperature for 1 hr. An additional portion of NABH(OAc)₃ (0.60 g) was added, and the mixture was stirred at room temperature for 1 hr. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 5→50% ethyl acetate/hexane) to give the title compound as a white amorphous solid (0.83 g, 82%).

MS(ESI+): 477(M−^{t}Bu+2H)

### Example 18

### (3S,4R)-N-{2-methoxy-5-[5-(trifluoromethyl)-1H-tetrazol-1-yl]benzyl}-3-phenylpiperidine-4-amine monohydrochloride

In the same manner as in Example 4 and using the compound obtained in Example 17, the title compounds was obtained as colorless crystals.

MS(ESI+): 433(M−HCl+H)

### Example 19

### N-{2-[(3S,4R)-4-({2-methoxy-5-[5-(trifluoromethyl)-1H-tetrazol-1-yl]benzyl}amino)-3-phenylpiperidin-1-yl]-2-oxoethyl}acetamide

To a solution of the compound (2.0 g) obtained in Example 18, Et₃N (0.44 g) and N-acetylglycine (0.49 g) in DMF (20 mL) were added WSC•HCl (1.2 g) and HOBt•H₂O (0.98 g), and the mixture was stirred at room temperature for 14 hrs. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 50→100% ethyl acetate/hexane) to give the title compound as colorless crystals.

MS(ESI+): 532 (M+H)

Optical rotation: [α]_{D}²⁵ −73.3° (c 1.0, MeOH)

Melting point: 114-116° C.

¹H-NMR(300 MHz,CDCl₃): δ 1.60-1.75. (1H, m), 1.95-1.99 (1H, m), 2.03 (3H×½, s), 2.05 (3H×½, s), 2.95-3.10 (2H, m), 3.25-3.70&3.79-4.19 (total 11H, m), 4.25-4.40 (1H×½, m), 4.53-4.58 (1H×½, m), 6.60-6.70 (1H, m), 6.84 (1H, dd, J=9.0, 3.0 Hz), 7.00-7.08 (3H, m), 7.18-7.28 (4H, m)

### Example 20

### N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide (crystal Form A)

To a solution of the compound (69.5 g) obtained in Example 4, Et₃N (15.0 g) and N-acetylglycine (17.0 g) in DMF (855 mL) were added WSC•HCl (42.6 g) and HOBt•H₂O (34.0 g), and the mixture was stirred at room temperature for 14 hrs. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 50→100% ethyl acetate/hexane) to give the title compound as colorless crystals (68.7 g, 87%).

MS(ESI+): 532 (M+H)

Optical rotation: [α]_{D}²⁵ +70.6° (c 1.0, MeOH)

Melting point: 114-116° C.

¹H-NMR(300 MHz,CDCl₃): δ 1.60-1.75 (1H, m), 1.95-1.99 (1H, m), 2.03 (3H×½, s), 2.05 (3H×½, s), 2.95-3.10 (2H, m), 3.25-3.70&3.79-4.19 (total 11H, m), 4.25-4.40 (1H×½, m), 4.53-4.58 (1H×½, m), 6.60-6.70 (1H, m), 6.84 (1H, dd, J=9.0, 3.0 Hz), 7.00-7.08 (3H, m), 7.18-7.28 (4H, m)

Powder X-ray diffraction: lattice spacing (d value, approximate); 5.83, 5.17, 4.61, 4.00, 3.40 angstroms

The compounds described in Examples 1-20 are as follows (Tables 4-6).

TABLE 4

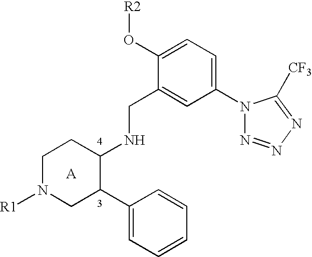

| Ex. No. | R1 | R2 | 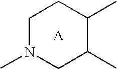 | additives | MS (ESI) |
|---|---|---|---|---|---|
| 1 ((+)-form) | 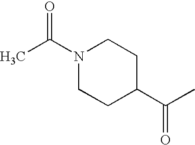 | CH₃ | 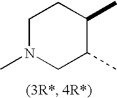 | HCl | 586 (M − HCl + H) |
| 2 ((−)-form) | 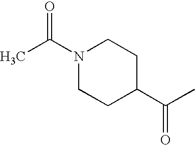 | CH₃ | 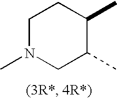 | HCl | 586 (M − HCl + H) |
| 3 | 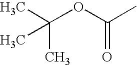 | CH₃ | 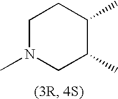 | | 477 (M − ^{t}Bu + 2H) |
| 4 | H | CH₃ | 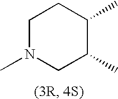 | HCl | 433 (M − HCl + H) |
| 5 | 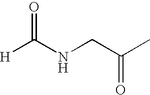 | CH₃ | 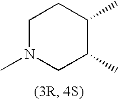 | | 518 (M + H) |
| 6 | 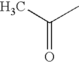 | CH₃ | 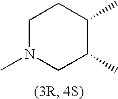 | HCl | 475 (M − HCl + H) |
| 7 | 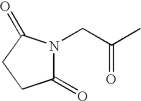 | CH₃ | 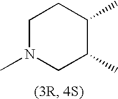 | HCl | 572 (M − HCl + H) |
| 8 | 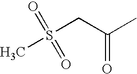 | CH₃ | 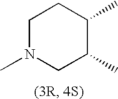 | HCl | 553 (M − HCl + H) |
| 9 | 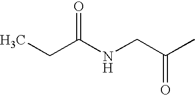 | CH₃ | 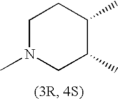 | HCl | 546 (M − HCl + H) |

TABLE 5

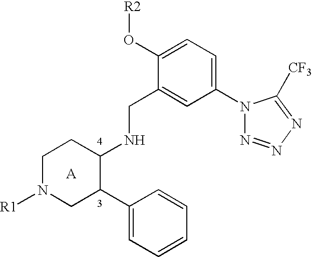

| Ex. No. | R1 | R2 | 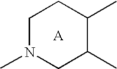 | additives | MS (ESI) |
|---|---|---|---|---|---|
| 10 | 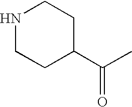 | CH₃ | 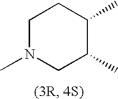 | 2HCl | 544 (M − 2HCl + H) |
| 11 | 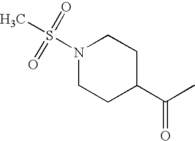 | CH₃ | 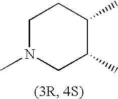 | HCl | 622 (M − HCl + H) |
| 12 (shorter Rt) | 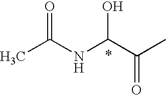 | CH₃ | 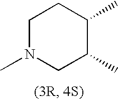 | | 548 (M + H) |
| 13 (longer Rt) | 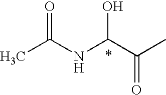 | CH₃ | 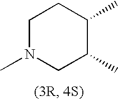 | | 548 (M + H) |
| 14 | 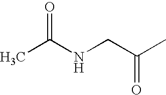 | H | 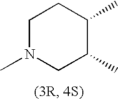 | | 518 (M + H) |
| 15 ((+)−form) | 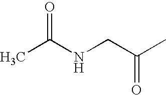 | CH₃ | 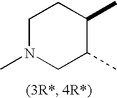 | HCl | 532 (M − HCl + H) |
| 16 ((−)−form) | 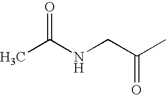 | CH₃ | 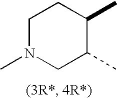 | HCl | 532 (M − HCl + H) |
| 17 | 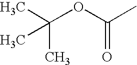 | CH₃ | 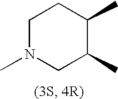 | | 477 (M − ^{t}Bu + 2H) |
| 18 | H | CH₃ | 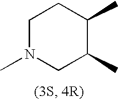 | HCl | 433 (M − HCl + H) |

TABLE 6

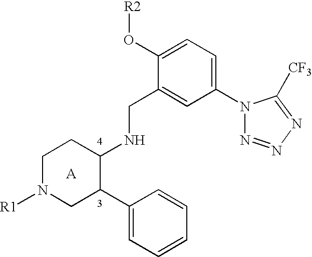

| Ex. No. | R1 | R2 | 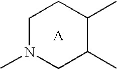 | additives | MS (ESI) |
|---|---|---|---|---|---|
| 19 | 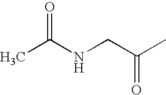 | CH₃ | 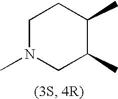 | | 532 (M + H) |
| 20 | 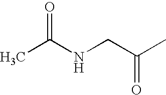 | CH₃ | 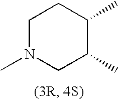 | | 532 (M + H) |

### Reference Example 29

### ethyl 3-(2-ethoxycarbonylethylamino)-2-phenylpropionate phosphate

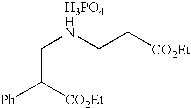

To ethyl phenylacetate (50 g, 305 mmol) were added dimethylformamide dimethylacetal (40 g, 335 mmol) and DMF (400 mL). After stirring at a bath temperature of 140° C. for 6 hrs, β-alanine ethyl ester hydrochloride (51.5 g, 335 mmol) was added to the reaction mixture, and the mixture was stirred at a bath temperature of 80° C. for 2 hrs. Under ice-cooling, acetic acid (400 mL) was added, and sodium triacetoxyborohydride (232.7 g, 1.098 mol) was added over about 15 min. at 25° C. After stirring at 50° C. for 2 hrs, ethyl acetate (800 mL) was added. Under ice-cooling, a 5N solution of sodium hydroxide (2 L) was slowly added paying attention to foam formation. After partitioning, the organic layer was washed twice with water (800 mL), and concentrated. The residue was dissolved in 2-propanol (200 mL) and phosphoric acid (30.0 g, 305 mmol) was added. The mixture was stirred at room temperature for 2 hrs, and stirred for 1 hr under ice-cooling, and the precipitated crystals were collected by filtration under reduced pressure and washed twice with 2-propanol (25 mL). The crystals were dried at 50° C. for 6 hrs to give the title compound (62.4 g) as white crystals.

¹H-NMR(300 MHz,DMSO-d₆): δ 1.11-1.21 (m, 6H), 2.74-2.85 (m, 2H), 3.14-3.24 (m, 3H), 3.39-3.46 (m, 1H), 4.05-4.18 (m, 4H), 4.26-4.30 (m, 1H), 7.15-7.29 (m, 9H).

Elemental analysis: C₁₆H₂₆NO₈P

Found C, 48.88; H, 6.58; N, 3.33; P, 7.61.

Calculated C, 49.10; H, 6.70; N, 3.58; P, 7.91.

### Reference Example 30

### 3-phenylpiperidin-4-one monohydrochloride

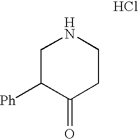

Ethyl 3-(2-ethoxycarbonylethylamino)-2-phenylpropionate phosphate (3.3 g, 8.5 mmol) was suspended in tetrahydrofuran (330 mL) and sodium tert-butoxide (4.5 g, 46.8 mmol) was added under ice-cooling. After stirring under ice-cooling for 30 min, the mixture was stirred at room temperature for 3 hrs. Water (44 mL) was added, and the mixture was stirred at 80° C. for 5 hrs and cooled. The mixture was extracted with ethyl acetate (44 mL), the aqueous layer was extracted with ethyl acetate (22 mL), and the organic layers were combined. The obtained brown solution was concentrated at 40-50° C., dissolved in ethanol (20 mL) and concentrate to dryness twice at 40-50° C. The residue was dissolved in ethanol (6.7 mL) and ethyl acetate (30 mL), and 4N hydrogen chloride/ethyl acetate solution (2.1 mL, 8.5 mmol) was added dropwise at room temperature. The mixture was heated at 80° C. for 4 hrs, crystallized, allowed to cool and stirred under ice-cooling stirred for 1 hr. The precipitated crystals were collected by filtration under reduced pressure, and washed twice with ethyl acetate (5 mL). Vacuum drying at 60° C. for 6 hrs gave the title compound (0.78 g) as pale-yellow crystals.

¹H-NMR(300 MHz,DMSO-d₆): δ2.53 (d, 2H), 2.96-3.07 (m, 1H), 3.52 (dt, 1H), 3.62 (d, 2H), 4.23 (t, 1H), 7.20-7.22 (m, 2H), 7.28-7.39 (m, 3H), 9.90 (brs, 2H).

### Reference Example 31

### 2-(cyclopropylmethoxy)-5-[5-(trifluoromethyl)-1H-tetrazol-1-yl]benzaldehyde

A solution of 2-hydroxy-5-[5-(trifluoromethyl)-1H-tetrazol-1-yl]benzaldehyde (1.00 g) (synthesized by a known method (WO95/08549 A1)), (bromomethyl)cyclopropane (0.75 mL), sodium iodide (1.16 g) and potassium carbonate (1.18 g) in DMF (10 mL) was stirred at 90° C. for 4 hrs. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 10→20% ethyl acetate/hexane) to give the title compound (1.04 g, 86%) as a white powder.

Melting point: 80-82° C.

### Reference Example 32

### 2-(trifluoromethoxy)-5-[5-(trifluoromethyl)-1H-tetrazol-1-yl]benzaldehyde

(Step 1)

To a solution of 3-bromo-4-(trifluoromethoxy)aniline (14.5 g) and Et₃N (7.3 g) in CH₂Cl₂ (60 mL) was added trifluoroacetic anhydride (13.7 g) at 0° C., and the mixture was stirred at room temperature for 1 hr. The reaction mixture was poured into water, and the product was extracted with ethyl acetate. The organic layer was washed with a saturated solution of ammonium chloride and brine and dried, and the solvent was evaporated under reduced pressure. The obtained residue was crystallized from IPE and hexane to give N-[3-bromo-4-(trifluoromethoxy)phenyl]-2,2,2-trifluoroacetamide as a white powder (19.5 g, 98%).

Melting point: 64-66° C.

(Step 2)

To a solution of the compound (19.3 g) obtained in step 1 in CCl₄ (155 mL) was added triphenylphosphine (21.6 g), and the mixture was stirred at 95° C. for 20 hrs. The reaction mixture was concentrated under reduced pressure and the obtained residue was dissolved in DMF (35 mL). The DMF solution was added to a suspension of sodium azide (5.7 g) in DMF (105 mL) at 0° C., and the mixture was stirred at room temperature for 30 min. The reaction mixture was poured into water, and the product was extracted with ethyl acetate. The organic layer was washed with a saturated solution of ammonium chloride and brine and dried, and the solvent was evaporated under reduced pressure. The precipitate was filtered off and the obtained residue was purified by silica gel column chromatography (solvent gradient; 17→20% ethyl acetate/hexane) to give 1-[3-bromo-4-(trifluoromethoxy)phenyl]-5-(trifluoromethyl)-1H-tetrazole as a colorless oil (15.7 g, 76%).

MS(ESI+): 377, 379(M+H)

(Step 3)

To a solution of the compound (13.2 g) obtained in Step 2 and Zn (CN)₂ (4.1 g) in DMF (85 mL) was added tetrakis(triphenylphosphine)palladium (0) (Pd(PPh₃) 4) (2.02 g), and the mixture was stirred under an argon atmosphere at 110° C. for 25 hrs. The reaction mixture was concentrated under reduced pressure, and the residue was poured into water. The product was extracted with ethyl acetate, the organic layer was washed with a saturated solution of ammonium chloride and brine and dried, and the solvent was evaporated under reduced pressure. The obtained residue was purified by silica gel column chromatography (solvent gradient; 10→20% ethyl acetate/hexane) to give 2-(trifluoromethoxy)-5-[5-(trifluoromethyl)-1H-tetrazol-1-yl]benzonitrile as a white powder (7.08 g, 63%).

Melting point: 65-67° C.

(Step 4)

A mixed solution of the compound (4.85 g) obtained in Step 3, Raney-nickel (approx. 20 g) and sodium phosphinate monohydrate (NaH₂PO₂.H₂O) (15.0 g) in pyridine-acetic acid-water (2:1:1(v/v), 80 mL) was stirred at 40° C. for 1.5 hrs. The catalyst was filtered off, and the filtrate was acidified with 2N hydrochloric acid. The product was extracted with ethyl acetate, the organic layer was washed with 2N hydrochloric acid and brine and dried, and the solvent was evaporated under reduced pressure. The obtained residue was purified by silica gel column chromatography (solvent gradient; 10→33% ethyl acetate/hexane) to give the title compound as a white powder (3.04 g, 62%).

Melting point: 40-42° C.

MS(ESI+): 327(M+H)

### Example 21

### N-[2-oxo-2-(4-oxo-3-phenylpiperidin-1-yl)ethyl]acetamide

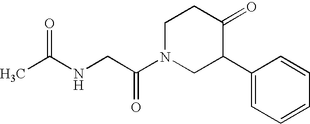

N-Acetylglycine (6.44 g) was suspended in acetonitrile (120 mL). 3-Phenylpiperidin-4-one monohydrochloride (10.58 g), triethylamine (5.06 g) and WSC•HCl (11.50 g) were successively added. The mixture was stirred at 50° C. for 2 hrs and cooled to 25° C. A 1:1 mixture of brine and 3N hydrochloric acid (40 mL) was added to partition the mixture. The aqueous layer was extracted again with acetonitrile (60 mL). The organic layers were combined, washed (X2) successively with a 1:1 mixture of brine and 5N sodium hydroxide (40 mL), and brine (40 mL). The organic layer was concentrated under reduced pressure, and azeotropically concentrated with ethyl acetate. Ethyl acetate (150 mL) and silica gel (10 g) were added to the residue, and the mixture was heated to 70° C. and stirred for 30 min. The hot silica-gel mixture was filtered and washed twice with ethyl acetate (100 mL). The filtrate was concentrated under reduced pressure, and azeotropically concentrated with toluene. Toluene (100 mL) was added to the residue and the residue was dissolved under refluxing. The mixture was cooled to 25° C. and the precipitated crystals were collected by filtration, washed twice with toluene (20 mL), and dried under reduced pressure to give the title compound as white crystals (8.70 g).

¹H-NMR(300 MHz,CDCl₃): δ2.06-2.07 (3H, m), 2.61-2.69 (2H, m), 3.50-3.76 (3H, m), 3.94-4.28 (3H, m), 4.57-4.65 (1H, m), 6.56 (1H, br), 7.11-7.38 (5H, m).

MS(FAB): 275(M+H).

Elemental analysis: C₁₅H₁₈N₂O₃.0.5H₂O

Found C, 63.41; H, 6.58; N, 10.09.

Calculated C, 63.59; H, 6.76; N, 9.89.

### Example 22

### N-[2-oxo-2-((3R,4S)-3-phenyl-4-{[(1S)-1-phenylethyl]amino}piperidin-1-yl)ethyl]acetamide

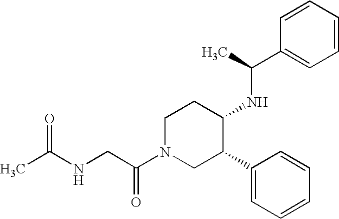

The compound (10 g) obtained in Example 21 was suspended in toluene (50 mL). (S)-1-Phenylethylamine (6.63 g), p-toluenesulfonic acid monohydrate (0.35 g) were successively added. The mixture was refluxed at 110° C. for 3 hrs using a Dean-Stark trap to remove water. The mixture was cooled to 25° C. Raney nickel catalyst (30 mL), ethanol (50 mL) and triethylamine (3.69 g) were added and the reduction was carried out at 50° C. under a hydrogen pressure of 0.5 to 1 MPa until absorption of hydrogen ceased. The reaction mixture was filtered by pressurization under a nitrogen stream and the Raney nickel catalyst washed twice with ethanol (10 mL). The filtrate was concentrated under reduced pressure. Water (100 mL) was added to the concentration residue and the mixture was refluxed for 30 min. After cooling to room temperature, a seed crystal was added and the mixture was stirred for 2 hrs. The precipitated crystals were collected by filtration, washed twice with water (50 mL) and dried under reduced pressure at 60° C. for 3 hrs to give the title compound as white crystals (11.64 g).

¹H-NMR(300 MHz,CDCl₃):δ 1.02-1.04 (3H, m), 1.52-1.64 (2H, m) 2.01-2.03 (3H, m), 2.96-3.07 (1H, m), 3.14-3.26 (1H, m), 3.36-3.54 (2H, m), 3.60-3.80 (1H, m), 3.84-3.91 (1H, m), 3.97-4.39 (2H, m), 6.61 (1H, br), 7.20-7.39 (10H, m).

MS(FAB): 380(M+H).

Elemental analysis: C₂₃H₂₉N₃O₂

Found C, 72.27; H, 7.59; N, 11.13.

Calculated C, 72.29; H, 7.70; N, 11.07.

### Example 23

### N-{2-[(3R,4S)-4-amino-3-phenylpiperidin-1-yl]-2-oxoethyl}acetamide

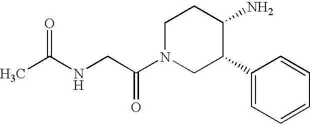

The compound (10 g) obtained in Example 22 was dissolved in ethanol (200 mL). 10% Palladium carbon (water-containing product) (5 g) was added. The reduction was carried out at 50° C. under a hydrogen pressure of 0.5 to 1 MPa until the absorption of hydrogen ceased. The reaction mixture was filtered and palladium carbon was washed twice with ethanol (20 mL). The filtrate was concentrated under reduced pressure to give the title compound (7.00 g).

¹H-NMR(300 MHz,CDCl₃): δ 1.70-1.77 (1H, m), 1.83-1.95 (1H, m), 2.03-2.05 (3H, m), 2.88-2.93 (1H, m), 3.30-3.96 (4H, m), 4.01-4.13 (2H, m), 4.25-4.51 (1H, m), 6.65 (1H, br), 7.15-7.37 (5H, m).

MS(FAB): 276(M+H).

Elemental analysis: C₁₅H₂₁N₃O₂

Found C, 65.04; H, 7.98; N, 15.00.

Calculated C, 65.43; H, 7.69; N, 15.26.

Chemical purity: 98.8%

Diastereomer excess: 98.8% de

Enantiomer excess: 94.8% ee

### Example 24

### N-{2-[(3R,4S)-4-amino-3-phenylpiperidin-1-yl]-2-oxoethyl}acetamide methanesulfonate

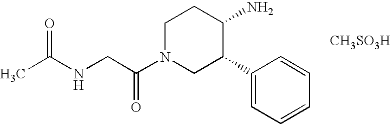

Ethanol (75 mL) was added to dissolve the compound (7.00 g) obtained in Example 23 by refluxing for 30 min. After cooling to 65° C., methanesulfonic acid (2.53 g) was added. After cooling to 25° C., ethyl acetate (150 mL) was added. The precipitated crystals were collected by filtration, and washed twice with ethanol/ethyl acetate (1:3) (40 mL). The crystals were dried under reduced pressure to give white crystals (9.07 g). Thereto was added ethanol (75 mL) to dissolve the crystals by refluxing for 30 min. After cooling to 25° C., the mixture was stirred for 3 hrs, and ethyl acetate (150 mL) was added. The precipitated crystals were collected by filtration, and washed twice with ethanol/ethyl acetate (1:3) (40 mL) and dried under reduced pressure to give the title compound as white crystals (8.84 g).

¹H-NMR(300 MHz,DMSO-d₆): δ 1.83-1.91 (2H, m), 1.88 (3H, s), 2.33 (3H, s), 3.15 (1H, br), 3.58-4.06 (7H, m), 7.30-7.40 (5H, m), 7.78 (3H, br), 7.96-8.03 (1H, m).

MS (FAB): 372 (M+H)

Elemental analysis: C₁₆H₂₅N₃O₅S.1.5H₂O

Found C, 48.12; H, 7.00; N, 10.59; S, 8.27.

Calculated C, 48.23; H, 7.08; N, 10.55; S, 8.05.

Chemical purity: 99.5%

Diastereomer excess: 99.8% de

Enantiomer excess: 99.7% ee

### Example 25

### N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide

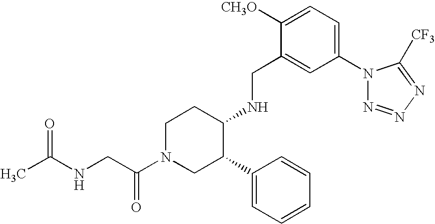

The compound (100 g) obtained in Example 24 was suspended in ethyl acetate (1 L). 2-Methoxy-5-[5-(trifluoromethyl)-1H-tetrazol-1-yl]benzaldehyde (73 g) was added, and then acetic acid (100 mL) and triethylamine (41 g) were added. The mixture was dissolved by stirring at 60° C. for 1 hr. After cooling to 5° C., NABH(OAc)₃ (114 g) was added. The mixture was stirred at 25° C. for 1 hr, cooled to 10° C., and 1N hydrochloric acid (500 mL) was added to partition the mixture. The aqueous layer was separated and the organic layer was further extracted twice with 1N hydrochloric acid (500 mL). The aqueous layers were combined, 5N solution of sodium hydroxide (1 L) was added at 10-20° C. After extraction with ethyl acetate (2 L), the organic layer was washed three times with water (1 L). The organic layer was concentrated under reduced pressure, and azeotropically concentrated twice with ethyl acetate (250 mL). The residue was dissolved in ethyl acetate (250 mL), filtered and washed with ethyl acetate (250 mL). Heptane (300 mL) and a seed crystal were added to the filtrate. The mixture was stirred at 25° C. for 21 hrs and then crystallized. Heptane (1.2 L) was added thereto, and the mixture was stirred for 1 hr. The precipitated crystals were collected by filtration, and washed twice with ethyl acetate/heptane (1:3) (400 mL). The mixture was dried at 60° C. for 6 hrs under reduced pressure to give the title compound as white crystals (126.6 g).

Melting point: 112-115° C.

Elemental analysis: C₂₅H₂₈N₇O₃F₃

Found C, 56.57; H, 5.38; N, 18.47; F, 10.70.

Calculated C, 56.49; H, 5.31; N, 18.45; F, 10.72.

Chemical purity: 99.7%

Diastereomer excess: 99.9% de

Enantiomer excess: 99.8% ee

### Example 26

### N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide

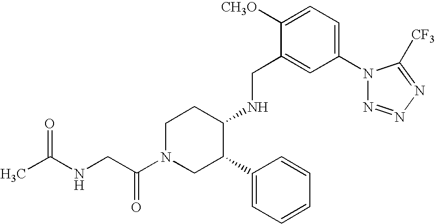

The compound (100 g) obtained in Example 24 was suspended in N,N-dimethylacetamide (100 mL) and ethyl acetate (200 mL). 2-Methoxy-5-[5-(trifluoromethyl)-1H-tetrazol-1-yl]benzaldehyde (73 g) and triethylamine (41 g) were added under a nitrogen stream, the mixture was stirred at room temperature for 15 min. and 10% palladium carbon (water-containing product) (10 g) was added. Under a hydrogen atmosphere, the mixture was stirred at room temperature for 4 hrs. The reaction mixture was diluted with ethyl acetate (400 mL), and the mixture was filtered under reduced pressure and washed with ethyl acetate (100 mL). Ethyl acetate (100 mL) was added to the filtrate, and the mixture was extracted with 1N hydrochloric acid (400 mL). Water (300 mL) was added to the organic layer and the mixture was further extracted. The extracts were combined, and ethyl acetate (1.5 L) and 5N solution of sodium hydroxide (100 mL) were added. After partitioning, the organic layer was washed three times with water (1 L). The organic layer was concentrated under reduced pressure, and further azeotropically concentrated with ethyl acetate (250 mL). The residue was dissolved in ethyl acetate (550 mL) and heptane (430 mL) was added. A seed crystal was added at 20° C. or below, and the mixture was stirred at room temperature for 4 hrs. Heptane (1 L) was added, and the mixture was stirred at room temperature for 2 hrs. The precipitated crystals were collected by filtration, and washed with ethyl acetate/heptane (1:2) (200 mL). The crystals were dried at 50° C. under reduced pressure to give the title compound as white crystals (123.3 g).

Melting point: 112-115° C.

Elemental analysis: C₂₅H₂₈N₇O₃F₃

Found C, 56.46; H, 5.25; N, 18.43.

Calculated C, 56.49; H, 5.31; N, 18.45.

### Example 27

### N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide (crystal Form B)

N-{2-[(3R,4S)-4-({2-Methoxy-5-[5-(trifluoromethyl)-1H-tetrazol-1-yl]benzyl}amino)-3-phenylpiperidin-1-yl]-2-oxoethyl}acetamide (2.0 g) was dissolved in tetrahydrofuran (2 mL) at 55° C. While stirring at 55° C., heptane (2 mL) was added, and the mixture was cooled to room temperature. After stirring at room temperature for 6 hrs, the precipitated crystal was collected by filtration and dried under reduced pressure at 50° C. to give the title compound as white crystals (1.5 g).

Melting point: 128-130° C.

Powder X-ray diffraction: lattice spacing (d value, approximate); 7.26, 4.61, 4.54, 4.38, 3.63 angstrom

### Example 28

### N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide (crystal Form B)

N-{2-[(3R,4S)-4-({2-Methoxy-5-[5-(trifluoromethyl)-1H-tetrazol-1-yl]benzyl}amino)-3-phenylpiperidin-1-yl]-2-oxoethyl}acetamide (2.0 g) was dissolved in tetrahydrofuran (2 mL) at 60° C. While stirring at 60° C., diisopropyl ether (2 mL) was added, and the mixture was cooled to room temperature. After stirring at room temperature for 14 hrs, the precipitated crystal was collected by filtration and dried under reduced pressure at 50° C. to give the title compound as white crystals (0.7 g).

Melting point: 128-130° C.

Powder X-ray diffraction: lattice spacing (d value, approximate); 7.26, 4.61, 4.54, 4.38, 3.63 angstrom

### Example 29

### N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide (crystal Form A)

N-{2-[(3R,4S)-4-({2-Methoxy-5-[5-(trifluoromethyl)-1H-tetrazol-1-yl]benzyl}amino)-3-phenylpiperidin-1-yl]-2-oxoethyl}acetamide (100 g) was dissolved in ethyl acetate (300 mL) at 50° C. Activated carbon (5 g) was added, and the mixture was stirred at 50° C. for 10 min. The activated carbon was filtered off and washed with ethyl acetate (100 mL). The filtrate was cooled to 20° C., heptane (80 mL) was added, and the mixture was stirred for 10 min. A seed crystal (0.05 g) having a melting point of about 115° C. was added at not more than 20° C., the mixture was stirred for 10 min. and an additional portion of heptane (160 mL) was added. The mixture was stirred at room temperature for 14 hrs, heptane (960 mL) was added, and the mixture was stirred at room temperature for 2 hrs. The precipitated crystal was collected by filtration and washed with ethyl acetate/heptane (1:3) (200 mL). The crystal was dried under reduced pressure at room temperature to give the title compound as white crystals (95.9 g).

Melting point: 112-115° C.

Powder X-ray diffraction: lattice spacing (d value, approximate); 5.83, 5.17, 4.61, 4.00, 3.40 angstrom

### Example 30

### N-{2-[(3R,4S)-4-({2-(cyclopropylmethoxy)-5-[5-(trifluoromethyl)-1H-tetrazol-1-yl]benzyl}amino)-3-phenylpiperidin-1-yl]-2-oxoethyl}acetamide monohydrochloride

To a solution of the compound (0.26 g) obtained in Example 24 and the compound (0.18 g) obtained in Reference Example 31 in acetic acid (0.1 mL) and CH₂Cl₂ (10 mL) was added NABH(OAc)₃ (0.44 g), and the mixture was stirred at room temperature for 1 hr. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 50→100% ethyl acetate/hexane) to give colorless oil (0.30 g, 75%). The obtained oil (0.30 g) was treated with 1 equivalent of 4N hydrogen chloride/ethyl acetate to give the title compound as a white powder (0.25 g).

MS(ESI+): 572 (M−HCl+H)

### Example 31

### N-{2-[(3R,4S)-4-({2-(cyclopropyloxy)-5-[5-(trifluoromethyl)-1H-tetrazol-1-yl]benzyl}amino)-3-phenylpiperidin-1-yl]-2-oxoethyl}acetamide monohydrochloride

To a solution of the compound (0.26 g) obtained in Example 24 and 2-(cyclopropyloxy)-5-[5-(trifluoromethyl)-1H-tetrazol-1-yl]benzaldehyde (0.18 g) synthesized by a known method (WO99/24423 A1) in acetic acid (0.10 mL) and CH₂Cl₂ (10 mL) was added NABH(OAc)₃ (0.44 g), and the mixture was stirred at room temperature for 1 hr. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 50→100% ethyl acetate/hexane) to give colorless oil (0.25 g, 63%). The obtained oil (0.25 g) was treated with 1 equivalent of 4N hydrogen chloride/ethyl acetate to give the title compound as a white powder (0.20 g).

MS(ESI+): 558(M−HCl+H)

### Example 32

### N-{2-oxo-2-[(3R,4S)-3-phenyl-4-({2-(trifluoromethoxy)-5-[5-(trifluoromethyl)-1H-tetrazol-1-yl]benzyl}amino)piperidin-1-yl]ethyl}acetamide

To a solution of the compound (0.41 g) obtained in Example 24 and the compound (0.33 g) obtained in Reference Example 32 in acetic acid (0.10 mL) and CH₂Cl₂ (8 mL) was added NABH(OAc)₃ (0.64 g), and the mixture was stirred at room temperature for 13 hrs. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 50→100% ethyl acetate/hexane) and preparative HPLC to give the title compound as a white amorphous solid (0.054 g, 9%).

MS(ESI+):586(M+H)

### Example 33

### tert-butyl (3R,4S)-3-phenyl-4-({2-(trifluoromethoxy)-5-[5-(trifluoromethyl)-1H-tetrazol-1-yl]benzyl}amino)piperidine-1-carboxylate

To a solution of tert-butyl (3R,4S)-4-amino-3-phenylpiperidine-1-carboxylate (1.04 g) (synthesized by a known method (WO03/101964 A1)) and the compound (1.23 g) obtained in Reference Example 32 in CH₂Cl₂ (45 mL) was added titanium tetrachloride (0.36 g) at 0° C., and the mixture was stirred at room temperature for 2 hrs. The reaction mixture was concentrated under reduced pressure, and the residue was dissolved in MeOH (15 mL). NaBH₃CN (0.71 g) was added, and the mixture was stirred at room temperature for 1 hr. The reaction mixture was poured into water, and the product was extracted with ethyl acetate. The organic layer was washed with a saturated solution of sodium hydrogencarbonate and brine, and dried, and the solvent was evaporated under reduced pressure. The obtained residue was purified by silica gel column chromatography (solvent gradient; 5→100% ethyl acetate/hexane) to give the title compound as a colorless oil (1.06 g, 48%).

MS(ESI+): 587(M+H)

### Example 34

### (3R,4S)-3-phenyl-N-{2-(trifluoromethoxy)-5-[5-(trifluoromethyl)-1H-tetrazol-1-yl]benzyl}piperidine-4-amine dihydrochloride

The reaction and purification in the same manner as in Reference Example 15 using the compound (1.04 g) obtained in Example 33 gave the title compound as a white amorphous solid (1.06 g, 95%).

MS(ESI+): 487 (M−2HCl+H)

### Example 35

### 2-methyl-4-oxo-4-[(3R,4S)-3-phenyl-4-({2-(trifluoromethoxy)-5-[5-(trifluoromethyl)-1H-tetrazol-1-yl]benzyl}amino)piperidin-1-yl]butan-2-ol

The reaction and purification in the same manner as in Example 5 using the compound (0.45 g) obtained in Example 34 and β-hydroxyisovaleric acid (0.14 g) gave the title compound as a white amorphous solid (0.040 g, 9%).

MS(ESI+): 587(M+H)

### Example 36

### (3R,4S)-1-[(1-acetylpiperidin-4-yl)carbonyl]-3-phenyl-N-{2-(trifluoromethoxy)-5-[5-(trifluoromethyl)-1H-tetrazol-1-yl]benzyl}piperidine-4-amine

The reaction and purification in the same manner as in Example 5 using the compound (0.45 g) obtained in Example 34 and 1-acetylpiperidine-4-carboxylic acid (0.21 g) gave the title compound as a white amorphous solid (0.050 g, 10%).

MS(ESI+): 640(M+H)

### Example 37

### 4-{[(3R,4S)-3-phenyl-4-({2-(trifluoromethoxy)-5-[5-(trifluoromethyl)-1H-tetrazol-1-yl]benzyl}amino)piperidin-1-yl]carbonyl}piperidine-2,6-dione

The reaction and purification in the same manner as in Example 5 using the compound (0.34 g) obtained in Example 34 and 2,6-dioxo-4-piperidinecarboxylic acid (0.21 g) gave the title compound as a white amorphous solid (0.021 g, 53%).

MS(ESI+): 640(M+H)

### Example 38

### (3R,4S)-3-phenyl-1-(1H-tetrazol-1-ylacetyl)-N-{2-(trifluoromethoxy)-5-[5-(trifluoromethyl)-1H-tetrazol-1-yl]benzyl}piperidine-4-amine

The reaction and purification in the same manner as in Example 5 using the compound (0.34 g) obtained in Example 34 and 1H-tetrazole-1-acetic acid (0.12 g) gave the title compound as a white amorphous solid (0.15 g, 43%).

MS(ESI+): 597 (M+H)

### Example 39

### (3R,4S)-1-[(methylsulfonyl)acetyl]-3-phenyl-N-{2-(trifluoromethoxy)-5-[5-(trifluoromethyl)-1H-tetrazol-1-yl]benzyl}piperidine-4-amine

The reaction and purification in the same manner as in Example 5 using the compound (0.34 g) obtained in Example 34 and methanesulfonylacetic acid (0.12 g) gave the title compound as a white amorphous solid (0.043 g, 12%).

MS(ESI+): 607(M+H)

### Example 40

### 1-{2-oxo-2-[(3R,4S)-3-phenyl-4-({2-(trifluoromethoxy)-5-[5-(trifluoromethyl)-1H-tetrazol-1-yl]benzyl}amino)piperidin-1-yl]ethyl)pyrrolidine-2,5-dione

The reaction and purification in the same manner as in Example 5 using the compound (0.34 g) obtained in Example 34 and (2,5-dioxo-pyrrolidin-1-yl)acetic acid (0.14 g) gave the title compound as a white amorphous solid (0.142 g, 38%).

MS(ESI+): 626(M+H)

The compounds described in Examples 30-40 are as follows (Tables 7-8).

TABLE 7

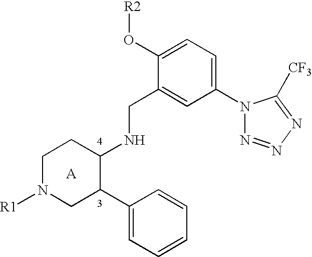

| Ex. No. | R1 | R2 | 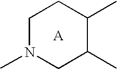 | additives | MS (ESI) |
|---|---|---|---|---|---|
| 30 | 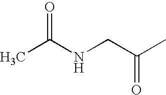 | 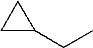 | 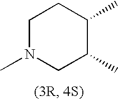 | HCl | 572 (M − HCl + H) |
| 31 | 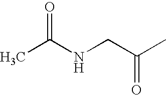 | 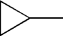 | 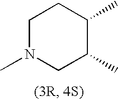 | HCl | 558 (M − HCl + H) |
| 32 | 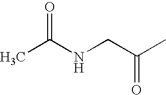 | CF₃ | 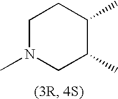 | | 586 (M + H) |
| 33 | 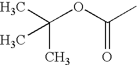 | CF₃ | 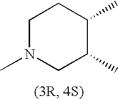 | | 587 (M + H) |
| 34 | H | CF₃ | 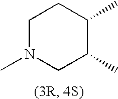 | 2HCl | 487 (M − 2HCl + H) |
| 35 | 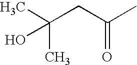 | CF₃ | 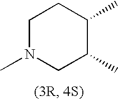 | | 587 (M + H) |
| 36 | 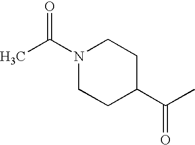 | CF₃ | 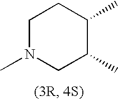 | | 640 (M + H) |
| 37 | 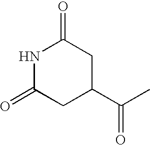 | CF₃ | 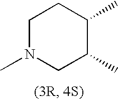 | | 626 (M + H) |
| 38 | 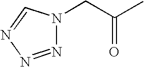 | CF₃ | 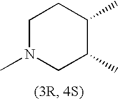 | | 597 (M + H) |

TABLE 8

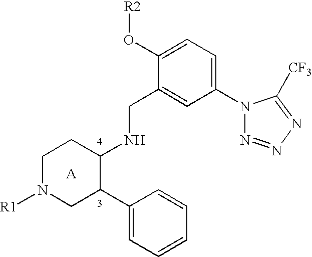

| Ex. No. | R1 | R2 | 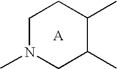 | additives | MS (ESI) |
|---|---|---|---|---|---|
| 39 | 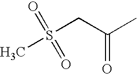 | CF₃ | 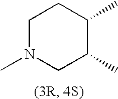 | | 607 (M + H) |
| 40 | 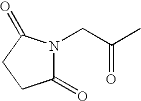 | CF₃ | 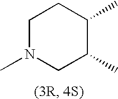 | | 626 (M + H) |

### Preparative Example 1

| | |
|---|---|
| (1) Compound of Example 1 | 10 mg |
| (2) Lactose | 60 mg |
| (3) Corn starch | 35 mg |
| (4) Hydroxypropylmethylcellulose | 3 mg |
| (5) Magnesium stearate | 2 mg |

A mixture of 10 mg of the compound obtained in Example 1, 60 mg of lactose and 35 mg of corn starch is granulated using 0.03 mL of an aqueous solution of 10 wt % hydroxypropylmethylcellulose (3 mg as hydroxypropylmethylcellulose), and then dried at 40° C. and sieved. The obtained granules are mixed with 2 mg of magnesium stearate and compressed. The obtained uncoated tablets are sugar-coated with an aqueous suspension of sucrose, titanium dioxide, talc and gum arabic. The thus-coated tablets are glazed with bees wax to obtain finally-coated tablets.

### Preparative Example 2

| | |
|---|---|
| (1) Compound of Example 1 | 10 mg |
| (2) Lactose | 70 mg |
| (3) Corn starch | 50 mg |
| (4) Soluble starch | 7 mg |
| (5) Magnesium stearate | 3 mg |

The compound (10 mg) obtained in Example 1 and 3 mg of magnesium stearate are granulated with 0.07 mL (7 mg as soluble starch) of an aqueous solution of soluble starch, dried, and mixed with 70 mg of lactose and 50 mg of corn starch. The mixture is compressed to obtain tablets.

### Reference Preparative Example 1

| | | |
|---|---|---|
| (1) Rofecoxib | 5.0 | mg |
| (2) Table salt | 20.0 | mg |
| (3) Distilled water | to 2 | mL of total volume |

Rofecoxib (5.0 mg) and 20.0 mg of table salt are dissolved in distilled water, and water is added to make 2.0 mL of total volume. The solution is filtered, and filled into 2 mL of ampoule under sterile condition. The ampoule is sterilized, and then sealed to obtain a solution for injection.

### Reference Preparative Example 2

| | | |
|---|---|---|
| (1) Rofecoxib | 50 | mg |
| (2) Lactose | 34 | mg |
| (3) Corn starch | 10.6 | mg |
| (4) Corn starch (paste) | 5 | mg |
| (5) Magnesium stearate | 0.4 | mg |
| (6) Calcium carboxymethylcellulose | 20 | mg |
| total | 120 | mg |

The above-mentioned (1) to (6) are mixed according to a conventional method and the mixture was tableted by a tablet machine to obtain tablets.

### Preparative Example 3

The formulation prepared in Preparative Example 1 or 2, and the formulation prepared in Reference Preparative Example 1 or 2 are combined.

### Experimental Example 1

Radioligand receptor binding inhibitory activity (Binding inhibitory activity using receptor from human lymphoblast cells (IM-9))

The method of M. A. Cascieri et al., “Molecular Pharmacology 42, p. 458 (1992)” was modified and used. The receptors were prepared from human lymphoblast cells (IM-9). IM-9 cells (2×10⁵ cells/mL) were incubated for 3 days (one liter), which were then subjected to centrifuge for 5 min. at 500×G to obtain cell pellets. The obtained pellets were washed once with phosphate buffer (Flow Laboratories, CAT. No. 28-103-05), which were then homogenized using Polytron homogenizer (“Kinematika”, Germany) in 30 mL of 50 mM Tris-HCl buffer (pH 7.4) containing 120 mM sodium chloride, 5 mM potassium chloride, 2 μg/mL chymostatin, 40 μg/mL bacitracin, 5 μg/mL phosphoramidon, 0.5 mM phenylmethylsulfonyl fluoride, 1 mM ethylenediamine tetra-acetate, which was subjected to centrifuge at 40,000×G for 20 min. The residue was washed twice with 30 mL of the buffer, which was then preserved frozen (−80° C.) as a specimen of the receptors.

The specimen was suspended in a reaction buffer (50 mM Tris-HCl buffer (pH 7.4), 0.02% bovine serum albumin, 1 mM phenylmethylsulfonyl fluoride, 2 μg/mL chymostatin, 40 μg/mL bacitracin and 3 mM manganese chloride) to have protein in the concentration of 0.5 mg/mL of protein and 100 μL portion of the suspension was used in the reaction. After addition of the sample and ¹²⁵I-BHSP (0.46 KBq), the reaction was allowed to proceed in 0.2 mL of reaction buffer at 25° C. for 30 min. The amount of nonspecific binding was determined by adding substance P at a final concentration of 2×10⁻⁶ M.

After the reaction, using a cell harvester (290 PHD, Cambridge Technology, Inc, U.S.A.), the reaction solution was filterd through a glass filter (GF/B, Whatman, U.S.A.), which was immersed in 0.1% polyethyleneimine for 24 hrs. and dried. After washing three times with 250 μL of 50 mM Tris-HCl buffer (pH 7.4) containing 0.02% bovine serum albumin, the radioactivity remaining on the filter was determined with a gamma counter.

The antagonistic activity of each compound obtained in Examples was determined in terms of the concentration necessary to cause 50% inhibition (IC₅₀ value) under the above-described conditions, and the results were shown in Table 9.

TABLE 9

| Example No. | IC₅₀ value (nM) |
|---|---|
| 5 | 0.017 |
| 6 | 0.026 |
| 7 | 0.015 |
| 8 | 0.022 |
| 9 | 0.012 |
| 10 | 0.016 |
| 11 | 0.021 |
| 12 | 0.016 |
| 13 | 0.062 |
| 20 | 0.017 |

The radio ligand means substance P labeled with [¹²⁵I]. From the Table 9, it has been clarified that the compounds of the present invention have superior antagonistic action for the substance P receptor.

### Experimental Example 2

Bladder Capacity Increasing Activity of Tachykinin Receptor Antagonist, Oxybutynin and Tolterodine (Bladder Capacity Increasing Action in Urethane Anesthetized Guinea Pigs)

A urinary frequency/urinary incontinence suppressing effect of a substance having antagonistic action for tachykinin receptors was shown in terms of the ability to increase bladder capacity in urethane anesthetized male guinea pigs and compared with that of oxybutynin and tolterodine, which are therapeutic drugs for overactive bladder. Using male guinea pig under anesthesia, after emptying the bladder by suction, saline was infused into the bladder at a constant rate (0.3 mL/min.) until voiding. This procedure was repeated to confirm stable bladder capacity (amount of saline injected before induction of voiding). After confirming a stable response, a compound dissolved in DMSO was intravenously administered and the action was measured. Changes in the bladder capacity and voiding pressure after drug administration were measured. The results are shown in Table 10. The compound of Example 20, which is a tachykinin receptor antagonist, increased the bladder capacity in a dose-dependent manner without affecting the voiding pressure. While both oxybutynin and tolterodine significantly increased the bladder capacity, they showed lower voiding pressure, and the voiding pressure lowering action of tolterodine was significant.

TABLE 10

| | | | changes in | changes in |
|---|---|---|---|---|
| | | | bladder | voiding |
| | | | capacity | pressure |
| | | | (vs. before | (vs. before |
| | dose | | administration, | administration, |
| drug | (mg/kg) | n | %) | %) |
| DMSO | | 6 | 0.0 ± 4.7 | 4.4 ± 4.0 |
| compound of | 0.01 | 6 | 5.8 ± 11.1 | 10.1 ± 8.4 |
| Example 20 | 0.03 | 6 | 3.0 ± 8.2 | 18.5 ± 18.5 |
| | 0.1 | 6 | 42.9 ± 6.0* | 3.0 ± 8.0 |
| | 0.3 | 6 | 71.2 ± 25.2* | 4.9 ± 11.0 |
| DMSO | | 7 | 0.0 ± 3.9 | 10.0 ± 7.1 |
| oxybutynin | 0.1 | 6 | 1.8 ± 7.0 | 4.3 ± 7.5 |
| | 0.3 | 6 | 19.3 ± 5.1^{#} | −11.6 ± 5.4 |
| | 1 | 6 | 43.6 ± 7.7^{#} | −12.1 ± 8.8 |
| | 3 | 6 | 36.8 ± 6.6^{#} | −6.0 ± 5.6 |
| DMSO | | 8 | 0.0 ± 4.3 | 12.7 ± 7.7 |
| tolterodine | 1 | 8 | −3.4 ± 4.4 | 0.1 ± 5.7 |
| | 3 | 8 | 14.8 ± 7.6 | −6.7 ± 6.5^{#} |
| | 10 | 8 | 31.7 ± 9.1^{#} | −15.5 ± 4.6^{#} |

The data shows mean ± standard error.
*P = 0.025, (significanct difference relative to DMSO administration control group, Shirley-Williams test, one-tailed).
^{#}P = 0.025, (significant difference relative to DMSO administration control group, Williams' test, one-tailed).

### INDUSTRIAL APPLICABILITY

The compound (I) and a crystal thereof are useful as pharmaceutical agents, such as tachykinin receptor antagonists, agents for lower urinary tract symptoms and the like.

This application is based on a patent application No. 2005-124334 filed in Japan, the contents of which are hereby incorporated by reference.

1. An optically active compound represented by the formula: (I): 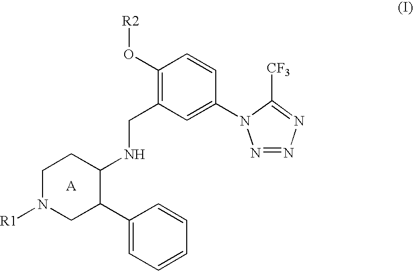
wherein
ring A is an optionally further substituted piperidine ring,
R1 is a hydrogen atom or a group represented by
R1′-C(═O)—
wherein R1′ is
(i) an optionally substituted 5- or 6-membered nitrogen-containing heterocyclic group,
(ii) an optionally substituted C₁₋₆ alkyl group, or
(iii) an optionally substituted C₁₋₆ alkoxy group, and
R2 is a hydrogen atom, an optionally substituted C₁₋₃ alkyl group, or a C₃₋₆ cycloalkyl group, except cis-1-(methoxyacetyl)-N-[2-methoxy-5-[5-(trifluoromethyl)-1H-tetrazol-1-yl]benzyl]-3-phenyl-4-piperidinamine and cis-1-[(1-acetyl-4-piperidinyl)carbonyl]-N-[2-methoxy-5-[5-(trifluoromethyl)-H-tetrazol-1-yl]benzyl]-3-phenyl-4-piperidinamine,
or a salt thereof.

2. The compound of claim 1, wherein R1 is a hydrogen atom or a group represented by R1′-C(═O)—
wherein R1′ is
(i) an optionally substituted 5- or 6-membered nitrogen-containing heterocyclic group,
(ii) an optionally substituted C₁₋₆ alkyl group, or
(iii) an optionally substituted C₁₋₆ alkoxy group,
except a methoxymethyl group and a 1-acetylpiperidin-4-yl group, and
R2 is a hydrogen atom, a C₁₋₃ alkyl group or a C₃₋₆ cycloalkyl group.

3. The compound of claim 1, which is a compound represented by the formula (I-A): 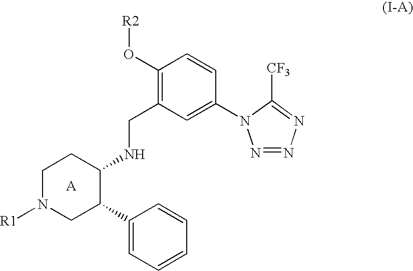
wherein each symbol is as defined in claim 1.

4. The compound of claim 1, which is a compound represented by the formula (Ia-A): 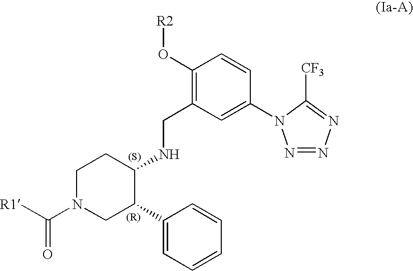
wherein
R1′ is
(i) a 5- or 6-membered nitrogen-containing heterocyclic group optionally having C₁₋₆ alkylsulfonyl group(s),
(ii) a C₁₋₆ alkyl group optionally having 1 to 3 substituents selected from
(1) —NR3R4
wherein
R3 is
(a) a hydrogen atom or
(b) a C₁₋₆ alkyl group optionally having oxo group(s), and
R4 is a hydrogen atom, or
R3 and R4 in combination optionally form a 5- to 7-membered ring optionally having oxo group(s),
(2) a C₁₋₆ alkylsulfonyl group,
(3) a hydroxy group and
(4) an oxo group, or
(iii) a C₁₋₆ alkoxy group, and
R2 is a hydrogen atom, methyl or trifluoromethyl.

5. N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide or a salt thereof.

6. A crystal of N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide or a salt thereof.

7. A crystal of N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide.

8. The crystal of claim 7, which has a melting point of not less than 90° C.

9. The crystal of claim 7, wherein the melting point is about 107° C. to about 119° C.

10. The crystal of claim 7, wherein the melting point is about 124° C. to about 134° C.

11. The crystal of claim 9, showing a diffraction pattern having characteristic peaks of lattice spacing (d value) at about 5.83, about 5.17, about 4.61, about 4.00 and about 3.40 angstroms by powder X-ray diffraction.

12. The crystal of claim 10, showing a diffraction pattern having characteristic peaks of lattice spacing (d value) at about 7.26, about 4.61, about 4.54, about 4.38 and about 3.63 angstroms by powder X-ray diffraction.

13. A pharmaceutical agent comprising the compound of claim 1.

14. The pharmaceutical agent of claim 13, which is a tachykinin receptor antagonist.

15. The pharmaceutical agent of claim 13, which is an agent for the prophylaxis or treatment of lower urinary tract disease, gastrointestinal disease or central nervous system disease.

16. The pharmaceutical agent of claim 13, which is an agent for the prophylaxis or treatment of lower urinary tract disease associated with overactive bladder and benign prostatic hyperplasia, pelvic visceral pain, lower urinary tract disease associated with chronic prostatitis, lower urinary tract disease associated with interstitial cystitis, irritable bowel syndrome, inflammatory bowel disease, vomiting, nausea, depression, anxiety neurosis, anxiety or sleep disorder (insomnia).

17. A method for the prophylaxis or treatment of lower urinary tract disease, gastrointestinal disease or central nervous system disease in mammals, which comprises administering an effective amount of the compound of claim 1 to said mammals.

18. Use of the compound of claim 1, for the production of an agent for the prophylaxis or treatment of lower urinary tract disease, gastrointestinal disease or central nervous system disease.

19. A method of producing the compound of claim 4, which comprises subjecting a compound represented by the formula: 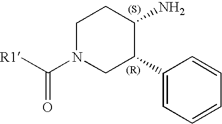
wherein each symbol is as defined in claim 4, or a salt thereof, to reductive alkylation with a compound represented by the formula: 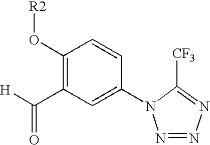
wherein each symbol is as defined in claim 4, or a salt thereof.

20. A method of producing the compound of claim 5, which comprises subjecting N-{2-[(3R,4S)-4-amino-3-phenylpiperidin-1-yl]-2-oxoethyl}acetamide or a salt thereof to reductive alkylation with 2-methoxy-5-[5-(trifluoromethyl)-1-H-tetrazol-1-yl]benzaldehyde or a salt thereof.

21. A method of producing the crystal of claim 9, which comprises bringing a solution of N-{2-[(3R,4S)-4-((2-methoxy-5-[5-(trifluoromethyl)-1H-tetrazol-1-yl]benzyl}amino)-3-phenylpiperidin-1-yl]-2-oxoethyl}acetamide or a salt thereof to supersaturation at less than 46° C. and performing crystal precipitation.

22. A method of producing the crystal of claim 10, which comprises bringing a solution of N-{2-[(3R,4S)-4-({2-methoxy-5-[5-(trifluoromethyl)-1H-tetrazol-1-yl]benzyl}amino)-3-phenylpiperidin-1-yl]-2-oxoethyl}acetamide or a salt thereof to supersaturation at not less than 46° C. and performing crystal precipitation.

23. N-{2-[(3R,4S)-4-amino-3-phenylpiperidin-1-yl]-2-oxoethyl}acetamide or a salt thereof.

24. A crystal of N-{2-[(3R,4S)-4-amino-3-phenylpiperidin-1-yl]-2-oxoethyl}acetamide methanesulfonate.

25. A method of producing a compound represented by the formula: 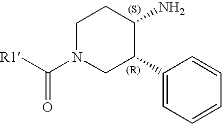
wherein R1′ is
(i) a 5- or 6-membered nitrogen-containing heterocyclic group optionally having C₁₋₆ alkylsulfonyl group(s),
(ii) a C₁₋₆ alkyl group optionally having 1 to 3 substituents selected from
(1) —NR3R4
wherein
R3 is
(a) a hydrogen atom or
(b) a C₁₋₆ alkyl group optionally having oxo group(s), and
R4 is a hydrogen atom, or
R3 and R4 in combination optionally form a 5- to 7-membered ring optionally having oxo group(s),
(2) a C₁₋₆ alkylsulfonyl group,
(3) a hydroxy group and
(4) an oxo group, or
(iii) a C₁₋₆ alkoxy group,
or a salt thereof, which comprises condensing a compound represented by the formula: 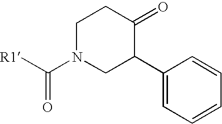
wherein R1′ is as defined above, with an optically active compound represented by the formula: 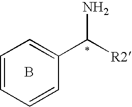
wherein ring B is an optionally fused benzene ring optionally having substituent(s), R2′ is a hydrocarbon group optionally having substituent(s), and * is an asymmetric center,
or a salt thereof, which is followed by hydrogenation and then hydrogenolysis.

26. A method of producing the compound of claim 23, which comprises condensing N-[2-oxo-2-(4-oxo-3-phenylpiperidin-1-yl)ethyl]acetamide with (S)-1-phenylethylamine or a salt thereof, hydrogenating the resulting compound to give N-[2-oxo-2-((3R,4S)-3-phenyl-4-{[(1S)-1-phenylethyl]amino}piperidin-1-yl)ethyl]acetamide or a salt thereof, and then hydrogenolyzing the compound.

27. N-[2-oxo-2-(4-oxo-3-phenylpiperidin-1-yl)ethyl]acetamide.

28. N-[2-oxo-2-((3R,4S)-3-phenyl-4-{[(1S)-1-phenylethyl]amino}piperidin-1-yl)ethyl]acetamide or a salt thereof.
